(19) United States
(12) Patent Application Publication
(10) Pub. No.: US 2023/0203176 A1
(43) Pub. Date: Jun. 29, 2023

(54) Methods For Prevention Of Graft Rejection In Xenotransplantation

(71) Applicants: Novartis AG, Basel (CH); Emory University, Atlanta, GA (US); The UAB Research Foundation, Birmingham, AL (US)

(72) Inventors: Guido JUNGE, Basel (CH); James RUSH, Basel (CH); Andrew B. ADAMS, Minneapolis, MN (US); Alfred Joseph TECTOR, III, Miami, FL (US)

(21) Appl. No.: 17/946,917

(22) Filed: Sep. 16, 2022

Related U.S. Application Data
(60) Provisional application No. 63/245,365, filed on Sep. 17, 2021.

(51) Int. Cl. C07K 16/28; A61K 39/395; A61K 45/06; C07K 16/18; A61P 37/06

(52) U.S. Cl. CPC C07K 16/2878; A61K 39/3955; A61K 45/06; C07K 16/18; A61P 37/06; C07K 2317/732; C07K 2317/76; C07K 2317/56; A61K 2039/545

(57) ABSTRACT

The present invention relates to methods, treatment regimens, uses, kits and therapies for prevention of graft rejection in solid organ transplantation, particularly solid organ xenotransplantation, by administering an anti-CD40 antibody or a combination of an anti-CD40 antibody and an anti-C5 antibody.

### CROSS-REFERENCE TO RELATED APPLICATIONS

This application claims the priority benefit of U.S. provisional application No. 63/245,365, filed Sep. 17, 2021, the contents of which are incorporated herein in their entireties by reference thereto.

### SEQUENCE LISTING

The instant application contains a Sequence Listing which has been submitted electronically in XML format and is hereby incorporated by reference in its entirety. Said XML copy, created on Oct. 6, 2022, is named NOV-024US_SL.xml and is 39,853 bytes in size.

### FIELD OF THE INVENTION

The present invention relates to methods, treatment regimens, uses, kits and therapies for prevention of graft rejection in solid organ transplantation, particularly solid organ xenotransplantation, by administering an anti CD40 antibody or a combination of an anti-C5 antibody and an anti CD40 antibody.

### BACKGROUND OF THE INVENTION

Allotransplantation of cells, tissues and organs has become a safe and effective method of treating end-stage organ failure. However, there is a growing shortage of available organs and as of Aug. 23, 2021 more than 14,000 people are on the active organ waiting list (see: https://www.eurotransplant.org/cms/). Solutions to address this problem include xenotransplantation, which typically involves the transplantation of animal cells, tissues, or organs into a human recipient. The domestic pig (Sus scrofa domestica) is similar both anatomically and physiologically to human and is currently considered the best donor of biological material for xenotransplantation. Advances in genetic engineering have made it possible to modify the genome of donor animals to reduce recognition of their organs by the human recipient's immune system.

In the context of allotransplantation, antibody mediated rejection (AMR) is associated with poor long term graft function and shorter graft survival in allotransplant recipients. In a pre-sensitized candidate who receives a human leucocyte antigen (HLA)-incompatible allograft, complement fixation and activation by donor specific antibodies (DSA) bound to allograft endothelium, leading to acute and chronic inflammation, vascular damage, and graft dysfunction, are a key mechanism of acute and subclinical AMR leading to subsequent allograft loss. A similar pathophysiology is observed in xenotransplantation with the consequence that AMR is a significant barrier to the clinical application of xenotransplantation, particularly renal xenotransplantation.

The main issue expected when using xenografts in human recipients is the immune response (e.g., rejection by antibody-mediated and cell-mediated responses). The main goal of genetic modification of pig xenograft organ donors is to create the least immunogenic organ possible and using the most optimal immunosuppression to prevent rejection of the transplanted xenograft. In the early 2000s, pigs with knockouts in the gene encoding α-1,3-galactosyltransferase have been generated by somatic cell nuclear transfer of engineered pig fibroblasts (PNAS 101, 7335-7340, 2004; Nat. Biotechnol. 20, 251-255, 2002; Science 295, 1089-1092, 2002). The authors of the publication J. Immunol. 193, 5751-5757, 2014 have eliminated all seven major histocompatibility complex (MHC) class I genes in pigs using the CRISPR-Cas9 technology. In the Scientific Report by Pin Li et al (Genetic engineering of porcine endothelial cell lines for evaluation of human-to-pig xenoreactive immune responses, (2021) 11:13131; https://doi.org/10.1038/s41598-021-92543-y) sequential disruption using CRISPR/Cas9 technology of five genes including GGTA1, CMAH, β4galNT2, SLA-I α chain, and β2-microglobulin in immortalized porcine endothelial cells was reported.

CD40 is a transmembrane glycoprotein constitutively expressed on B cells and antigen presenting cells (APCs) such as monocytes, macrophages, and dendritic cells (DC). CD40 is also expressed on platelets, and under certain conditions can be expressed on eosinophils, and parenchymal cells. The ligand for CD40 (CD154, CD40 ligand or CD40L), is inducible on a variety of cell types including activated T cells, platelets, and B cells.

Binding of CD154 to CD40 induces signaling via NF-KB, and MAPK pathways resulting in a variety of cell-type dependent activation outcomes. For example, signaling via this pathway is essential for several important effector functions of the adaptive immune system including primary T-cell-dependent antibody responses (TDARs), B cell proliferation, germinal center (GC) formation, immunoglobulin (Ig) isotype switching, somatic mutation, and differentiation of memory B and plasma cells. In addition to effects on B cells, CD40 pathway activation provides important signals for DC maturation and function, as well as monocyte and macrophage survival and cytokine secretion. More recently, CD40-CD154 pathway signaling has been implicated in the function of parenchymal cells in inflamed tissue, with activated epithelial cells from kidney, salivary gland and skin producing chemokines in response to CD40 ligation.

Therefore, the CD40-CD154 pathway is thought to play an important role in survival of grafts in organ transplantation and an antibody capable of blocking CD40-CD154 signalling, could be suitable for the prevention of graft loss in xenotransplantation. However, after promising results obtained in animal models, anti-CD154 antibodies were tested inter alia in patients undergoing renal transplantation. These trials showed efficacy but were halted when several patients suffered thromboembolic events (Boumpas D T, Furie R, Manzi S, et al. A short course of BG9588 (anti-CD40 ligand antibody) improves serologic activity and decreases hematuria in patients with proliferative lupus glomerulonephritis. Arthritis Rheum. 2003; 48:719-727). In Phase 1 and 2 trials conducted from 2001-2002 in patients with systemic lupus erythematosus, the agent proved safe but was not efficacious (Kalunian K C, Davis J C, Merrill J T, et al. Treatment of systemic lupus erythematosus by inhibition of T cell costimulation with anti-CD154: A randomized, double-blind, placebo-controlled trial. Arthritis Rheum. 2002; 46:3251-3258). Clinical use of anti-CD154mAb was put on hold. Thromboembolic events have also been reported in NHPs in association with use of anti-CD154mAb (Kawai T, Andrews D, Colvin R B, et al. Thromboembolic complications after treatment with monoclonal antibody against cd40 ligand. Nat Med. 2000; 6:114).

One of the most widely used anti-CD40 antibodies in preclinical xenotransplantation is the clone 2C10R4 which was developed using rhesus CD40 as the immunogen. Mohiuddin and co-workers (Mohiuddin M M, Singh A K, Corcoran P C, et al. Chimeric 2C10R4 anti-CD40 antibody therapy is critical for long-term survival of GTKO.hCD46.hTBM pig-to-primate cardiac xenograft. Nat Commun. 2016; 7:11138.) demonstrated that long-term survival of genetically-engineered pig heterotopic heart grafts could be achieved in NHPs. Genetic modifications in the pig combined with a treatment regimen based on the antibody 2C10R4 prevented humoral rejection and systemic coagulation pathway dysregulation, sustaining cardiac xenograft survival in one case beyond 900 days. Iwase and co-workers (Pig-to-baboon heterotopic heart transplantation-exploratory preliminary experience with pigs transgenic for human thrombomodulin and comparison of three costimulation blockade-based regimens. Xenotransplantation. 2015; 22:211-220. PubMed: 25847282) proved that the combination of anti-CD40mAb+belatacept was effective in preventing a T cell response in a pig-to-baboon heart xenotransplantation model. However, treatment with 2C10R4 was not as effective at prolonging kidney xenograft survival compared to a similar anti-CD154 based regimen, for unknown reasons.

The complement system and its components enhance the ability of antibodies and phagocytic cells to clear pathogens from an organism, thereby protecting against infection by linking adaptive and innate immunity as well as disposing of immune complexes and the products of inflammatory injury. Three clinical observations support a role of complement in AMR: 1) preformed complement-activating anti-donor antibodies confer a substantial risk for immediate immunologic graft loss; 2) capillary deposits of complement split-product C4d predict adverse transplant survival; and 3) complement-binding donor specific antibodies (DSA) detected in serum are associated with high rates of graft loss (Bohmig G A et al., (2018) Transplantation, 102(11): 1837-43).

C5 in particular has been demonstrated as a high-yield target for complement inhibition, as C5 activation activates chemotaxis (via C5a) and forms the initial component of the cell membrane attack complex (via C5b). Activation of complement and deposition of the membrane attack complex have been shown to directly initiate cell stimulatory, pro-coagulation, and pro-inflammatory responses. Previous studies in xenotransplantation have demonstrated that inhibition of complement activation, including antibody-deficiency, complement-deficiency, or complement-blocking agents, can prevent AMR in a mouse model (Rollins S A et al., (1995) Transplantation, 60(11): 1284-92). Complement inactivation has also been demonstrated to be beneficial in human-to-human allografts; in an uncontrolled prospective pilot study, crossmatch-positive renal allograft recipients were subjected to pre-emptive treatment with the anti-C5 antibody eculizumab resulting in early AMR rates that were by far lower than those documented in a historical control group of sensitized patients (7.7% versus 41%) (Stegall M D et al., (2011) Am J Transplant, 11(11): 2405-13). Interestingly anti-C5 therapy limited complement activation in an ex-vivo perfusion model of pig-to-primate heart xenotransplantation resulting in significantly prolonged survival times (Kroshus T J et al., (1995) Transplantation, 60(11): 1194-202). These data confirm the importance of complement in AMR and suggest that antibody therapy targeted against C5 could serve as a potent inhibitor of hyperacute/accelerated antibody mediated rejection in the pig-to-nonhuman primate xenotransplant model. Andrew Adams and co-workers have disclosed in the publication “Anti-C5 Antibody Tesidolumab Reduces Early Antibody-mediated Rejection and Prolongs Survival in Renal Xenotransplantation Ann. Surg. 2021 Sep. 1; 274(3):473-480, that temporary anti-C5 therapy reduced early graft loss secondary to antibody-mediated rejection and improved graft survival. Deleting class I MHC (SLA I) in donor pigs did not ameliorate early antibody-mediated rejection.

A further contribution to AMR in xenotransplant rejection is the generation of de novo antibodies specific to the graft once transplantation has occurred. By their very nature these antibodies cannot be screened for pre-graft and therefore suitable treatments are required for administration post graft, to prevent and reduce the generation of these de novo antibodies.

Further improvements to long-term xenograft survival and the prevention of antibody and complement-mediated damage will be important for the eventual clinical translation of xenotransplantation. It has been shown that long-term survival can be achieved in a pre-clinical pig to non-human primate kidney transplant model (Higginbotham L et al., (2015) Xenotransplantation 22(3): 221-30); Adams A B et al., (2018) Ann Surg. 268(4): 564-73). While the elimination of αGal and β4Gal via creation of the double knockout (DKO) pig eliminates two of the most important xenoantigens, there still remains some level of anti-pig antibody directed at other yet to be described antigens resulting in early transplant failure secondary to antibody mediated rejection in some recipients. Chemical immunosuppressive strategies using anti-proliferative agents e.g. mycophenolate mofetil (MMF), steroids e.g. prednisone and T cell immunosuppression e.g. calcineurin inhibitors such as cyclosporin and tacrolimus have been used in allotransplantation and xenotransplantation studies for many years. Unfortunately, anti-C5 antibody therapy did not allow for the use of tacrolimus instead of anti-CD154, prolonging survival to a maximum of 62 days with all grafts succumbing to AMR. There is a need for an improved immunosuppression strategy to prevent and treat AMR and to avoid negative effects in transplant recipients of a xeno organ, tissue, or cells. Hence, there is a significant need for improved methods to increase xenograft survival.

### SUMMARY OF THE INVENTION

It has been found that human, anti-CD40 monoclonal antibodies with silenced ADCC activity that bind both the xenograft organ CD40 and the human CD40, wherein said binding inhibits CD40L induced signaling with no or low agonist activity with respect to CD40 signaling, are suitable for the prevention of graft rejection in a subject receiving a xenograft organ. It has furthermore been found that a combination of CD40 signaling inactivation with inactivation of the complement system, in particular a combination of the above described CD40 antibody with an antibody targeted against C5 is particularly suitable for prevention of graft rejection in a subject receiving a xenograft organ.

In a first aspect, the invention relates to an anti-CD40 antibody or a functional fragment thereof for use in the prevention of graft rejection in a subject receiving a xenograft organ, wherein the anti-CD40 antibody is an anti-CD40 antibody, or a functional fragment thereof, with silenced ADCC activity that binds both the xenograft organ and the human CD40 and said binding inhibits CD40L induced signaling with no or low agonist activity with respect to CD40 signaling. The anti-CD40 antibody with silenced ADCC activity can comprise for example a silent Fc IgG1 region.

In an alternative first aspect, the invention relates to a pharmaceutical composition comprising an anti-CD40 antibody or a functional fragment thereof for use in the prevention of graft rejection in a subject receiving a xenograft organ, wherein the anti-CD40 antibody is an anti-CD40 antibody, or a functional fragment thereof, with silenced ADCC activity that binds both the xenograft organ and the human CD40 and said binding inhibits CD40L induced signaling with no or low agonist activity with respect to CD40 signaling. The anti-CD40 antibody with silenced ADCC activity can comprise for example a silent Fc IgG1 region.

In a first embodiment of the first aspects of the invention, the xenograft organ is from a pig and the anti-CD40 antibody binds the pig CD40.

In an alternative first embodiment of the first aspects of the invention, the anti-CD40 antibody is an anti-CD40 antibody or a functional fragment thereof with silenced ADCC activity that binds both the xenograft organ and the human CD40, wherein
a. the anti-CD40 antibody or a functional fragment thereof binds an epitope of the xenograft CD40 protein and the human CD40 protein, said epitope being comprised between amino acids 64-120 of SEQ ID NO:37 (human CD40 protein sequence, or the equivalent region in the pig CD40 protein); or
b. the anti-CD40 antibody or a functional fragment thereof binds to amino acids in the epitope region comprising amino acids (i) 60-70, (ii) 75-95 and (iii) 115-125 of SEQ ID NO:37 (or the equivalent region in the pig CD40 protein), or
c. the anti-CD40 antibody or a functional fragment thereof binds to an epitope consisting the amino acids (i) 64-75, (ii) 79-80, (iii) 82-87, (iv) 94 and (v) 118-120 of SEQ ID NO:37 (or the equivalent region in the pig CD40 protein) and said binding inhibits CD40L induced signaling with no or low agonist activity with respect to CD40 signaling.

In a second embodiment of the first aspects of the invention, the pig is a transgenic organism. In a third embodiment of the first aspects of the invention, alone or in combination with other embodiments of the first aspect, the transgenic donor pig comprises the following genetic modification: disrupted a(1,3)-galactosyltransferase and cytidine monophosphate-N-acetylneuraminic acid hydroxylase (CMAH) genes.

In an alternative third embodiment of the first aspects of the invention, the transgenic donor pig comprises disrupted a(1,3)-galactosyltransferase and CMAH genes and additional genetic modifications (e.g. as described below).

In a fourth embodiment of the first aspects of the invention, alone or in combination with other embodiments of the first aspects, the anti-CD40 antibody or a functional fragment thereof comprised in the pharmaceutical composition comprises an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30.

In a fifth embodiment of the first aspects of the invention, alone or in combination with other embodiments of the first aspects, the anti-CD40 antibody or a functional fragment thereof comprised in the pharmaceutical composition comprises an immunoglobulin VH domain comprising the amino acid sequence of SEQ ID NO: 31 and an immunoglobulin VL domain comprising the amino acid sequence of SEQ ID NO: 32.

In a sixth embodiment of the first aspects of the invention, alone or in combination with other embodiments of the first aspects, the anti-CD40 antibody or a functional fragment thereof comprised in the pharmaceutical composition comprises an immunoglobulin HC domain comprising the amino acid sequence of SEQ ID NO: 33 and an immunoglobulin LC domain comprising the amino acid sequence of SEQ ID NO: 34.

In an alternative sixth embodiment of the first aspects of the invention, alone or in combination with other embodiments of the first aspects, the anti-CD40 antibody or a functional fragment thereof comprised in the pharmaceutical composition is iscalimab.

In a seventh embodiment of the first aspects of the invention, alone or in combination with other embodiments of the first aspects, the anti-CD40 antibody or a functional fragment thereof comprised in the pharmaceutical composition comprises an immunoglobulin HC domain comprising the amino acid sequence of SEQ ID NO: 35 and an immunoglobulin LC domain comprising the amino acid sequence of SEQ ID NO: 36.

In an eighth embodiment of the first aspects of the invention, alone or in combination with other embodiments of the first aspects, the anti-CD40 antibody or a functional fragment thereof comprised in the pharmaceutical composition for use in the prevention of graft rejection in a subject receiving a xenograft organ is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 50 mg anti-CD40 antibody, or a functional fragment thereof per kg of the subject, followed by a maintenance dose which is between 300 mg and 600 mg.

In a ninth embodiment of the first aspects of the invention, alone or in combination with other embodiments of the first aspects, the loading dose of the anti-CD40 antibody or a functional fragment thereof, e.g. comprised in the pharmaceutical composition, for use in the prevention of graft rejection in a subject receiving a xenograft organ, is administered at a dose of about 10 mg/kg to about 50 mg/kg per antibody. In an alternative embodiment, said dose is about 10 mg/kg of the anti-CD40 antibody, or a functional fragment thereof on the day of xenograft organ.

In a tenth embodiment of the first aspects of the invention, alone or in combination with other embodiments of the first aspects, the subject receiving the anti-CD40 antibody or a functional fragment thereof, or the pharmaceutical composition comprising an anti-CD40 antibody, or a functional fragment thereof for use in the prevention of xenograft organ rejection is administered an induction therapy prior to receiving the xenotransplant organ. In an alternative embodiment, said induction therapy is administration of an anti-CD4 antibody and/or an anti-CD20 antibody.

In an eleventh embodiment of the first aspects of the invention, alone or in combination with other embodiments of the first aspects, the pharmaceutical composition for use in the prevention of graft rejection is combined with other anti-proliferative agents like mycophenolate mofetil (MMF), or steroids, like prednisone or T cell immunosuppressive compounds, like calcineurin inhibitors such as cyclosporin and tacrolimus.

In a second aspect the invention relates to a method of suppressing the rejection and prolonging the survival of a xenograft donor organ from an animal in a human recipient, the method comprising administering to the human recipient an anti-CD40 antibody, wherein the anti-CD40 antibody is an anti-CD40 antibody, or a functional fragment thereof, with silenced ADCC activity that binds both the xenograft organ and the human CD40 and said binding inhibits CD40L induced signaling with no or low agonist activity with respect to CD40 signaling. In a first embodiment of the second aspect of the invention, the xenograft organ is from a pig and the anti-CD40 antibody binds the pig CD40.

In an alternative first embodiment of the second aspect of the invention, the anti-CD40 antibody is an anti-CD40 antibody or functional fragment thereof with silenced ADCC activity that binds both the xenograft organ and the human CD40, wherein
a. the anti-CD40 antibody binds an epitope of the xenograft CD40 protein and the human CD40 protein, said epitope being comprised between amino acids 64-120 of SEQ ID NO:37 (human CD40 protein sequence, or the equivalent region in the xenograft CD40 protein); or
b. the anti-CD40 antibody binds to amino acids in the epitope region comprising amino acids (i) 60-70, (ii) 75-95 and (iii) 115-125 of SEQ ID NO:37 (or the equivalent region in the xenograft CD40 protein), or
c. the anti-CD40 antibody binds to an epitope consisting the amino acids (i) 64-75, (ii) 79-80, (iii) 82-87, (iv) 94 and (v) 118.-120 of SEQ ID NO:37 (or the equivalent region in the xenograft CD40 protein) and said binding inhibits CD40L induced signaling with no or low agonist activity with respect to CD40 signaling.

In a second embodiment of the second aspect of the invention, the pig is a transgenic organism.

In a third embodiment of the second aspect of the invention, the transgenic donor pig comprises the following genetic modifications as follows: disrupted a(1,3)-galactosyltransferase and CMAH genes.

In an alternative third embodiment of the second aspect of the invention, the transgenic donor pig comprises disrupted a(1,3)-galactosyltransferase and CMAH genes and additional genetic modifications.

In a fourth embodiment of the second aspect of the invention, alone or in combination with other embodiments of the second aspect, the anti-CD40 antibody or a functional fragment thereof comprises an immunoglobulin VH domain comprising the hypervariable regions set forth as S SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30.

In a fifth embodiment of the second aspect of the invention, alone or in combination with other embodiments of the second aspect, the anti-CD40 antibody or a functional fragment thereof comprises an immunoglobulin VH domain comprising the amino acid sequence of SEQ ID NO: 31 and an immunoglobulin VL domain comprising the amino acid sequence of SEQ ID NO: 32.

In a sixth embodiment of the second aspect of the invention, alone or in combination with other embodiments of the second aspect, the anti-CD40 antibody comprises an immunoglobulin HC domain comprising the amino acid sequence of SEQ ID NO: 33 and an immunoglobulin LC domain comprising the amino acid sequence of SEQ ID NO: 34.

In a seventh embodiment of the second aspect of the invention, alone or in combination with other embodiments of the second aspect, the anti-CD40 antibody comprises an immunoglobulin HC domain comprising the amino acid sequence of SEQ ID NO: 35 and an immunoglobulin LC domain comprising the amino acid sequence of SEQ ID NO: 36.

In an eighth embodiment of the second aspect of the invention, alone or in combination with other embodiments of the second aspect, the anti-CD40 antibody or a functional fragment thereof is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 50 mg anti-CD40 antibody or a functional fragment thereof per kg of the subject, followed by a maintenance dose which is between 300 mg and 600 mg.

In a ninth embodiment of the second aspect of the invention, alone or in combination with other embodiments of the second aspect, the loading dose of the anti-CD40 antibody or a functional fragment thereof is administered at a dose of about 10 mg/kg to about 50 mg/kg per antibody. In an alternative embodiment, said dose is about 10 mg/kg of the anti-CD40 antibody or a functional fragment thereof on the day of xenograft organ.

In a tenth embodiment of the second aspect of the invention, alone or in combination with other embodiments of the second aspect, the subject receiving the anti-CD40 antibody or a functional fragment thereof is administered an induction therapy prior to receiving the xenotransplant. In an alternative embodiment, said induction therapy is administration of an anti-CD4 antibody and/or an anti-CD20 antibody.

In an eleventh embodiment of the second aspect of the invention, alone or in combination with other embodiments of the second aspect, the method for suppressing the rejection and prolonging the survival of a xenograft organ comprising the administration of an anti-CD40 antibody or a functional fragment thereof in combination with other anti-proliferative agents like mycophenolate mofetil (MMF), or steroids, like prednisone or T cell immunosuppressive compounds, like calcineurin inhibitors such as cyclosporin and tacrolimus.

In a third aspect, the present invention relates to a pharmaceutical composition comprising an anti-C5 antibody, or the functional fragment thereof, and an anti-CD40 antibody, or the functional fragment thereof in combination with at least a pharmaceutical acceptable excipient, carrier or diluent.

In an alternative third aspect, the invention relates to a pharmaceutical combination comprising an anti-C5 antibody, or the functional fragment thereof, and an anti-CD40 antibody, or the functional fragment thereof, for simultaneous, sequential, or separate administration.

In a first embodiment of this third aspects of the invention, the CD40 antibody comprised in the pharmaceutical composition is an anti-CD40 antibody or functional fragment thereof with silenced ADCC activity that binds both the xenograft organ/transplant and the human CD40 and said binding inhibits CD40L induced signaling with no or low agonist activity with respect to CD40 signaling. The anti-CD40 antibody with silenced ADCC activity comprised in said pharmaceutical composition can comprise a silent Fc IgG1 region.

In a second embodiment of the third aspects of the invention, alone or in combination with other embodiments of the third aspects, the anti-CD40 antibody or a functional fragment thereof comprised in the pharmaceutical composition comprises an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30.

In a third embodiment of the third aspects of the invention, alone or in combination with other embodiments of the third aspects, the anti-CD40 antibody or a functional fragment thereof comprised in the pharmaceutical composition comprises an immunoglobulin VH domain comprising the amino acid sequence of SEQ ID NO: 31 and an immunoglobulin VL domain comprising the amino acid sequence of SEQ ID NO: 32.

In a fourth embodiment of the third aspects of the invention, alone or in combination with other embodiments of the third aspects, the anti-CD40 antibody comprised in the pharmaceutical composition comprises an immunoglobulin HC domain comprising the amino acid sequence of SEQ ID NO: 33 and an immunoglobulin LC domain comprising the amino acid sequence of SEQ ID NO: 34.

In a fifth embodiment of the third aspects of the invention, alone or in combination with other embodiments of the third aspects, the anti-CD40 antibody comprised in the pharmaceutical composition comprises an immunoglobulin HC domain comprising the amino acid sequence of SEQ ID NO: 35 and an immunoglobulin LC domain comprising the amino acid sequence of SEQ ID NO: 36.

In a sixth embodiment of the third aspects of the invention, alone or in combination with other embodiments of the third aspects, the anti-CD40 antibody is iscalimab.

In a seventh embodiment of the third aspects of the invention, alone or in combination with other embodiments of the third aspects, the anti-C5 antibody or a functional fragment thereof comprised in the pharmaceutical composition is an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6.

In an eights embodiment of the third aspects of the invention, alone or in combination with other embodiments of the third aspects, the anti-C5 antibody comprised in the pharmaceutical composition is an anti-C5 antibody or a functional fragment thereof comprising an immunoglobulin VH domain comprising the amino acid sequence of SEQ ID NO: 7 and an immunoglobulin VL domain comprising the amino acid sequence of SEQ ID NO: 8.

In a ninth embodiment of the third aspects of the invention, alone or in combination with other embodiments of the third aspects, the anti-C5 antibody comprised in the pharmaceutical composition is an anti-C5 antibody or a functional fragment thereof comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16.

In an tenth embodiment of the third aspects of the invention, alone or in combination with other embodiments of the third aspects, the anti-C5 antibody comprised in the pharmaceutical composition is an anti-C5 antibody comprising an immunoglobulin VH domain comprising the amino acid sequence of SEQ ID NO: 17 and an immunoglobulin VL domain comprising the amino acid sequence of SEQ ID NO: 18.

In an eleventh embodiment of the third aspects of the invention, alone or in combination with other embodiments of the third aspects, the anti-C5 antibody is tesidolumab or eculizumab.

In a fourth aspect the invention relates to a pharmaceutical composition according to the third aspect of the invention and all embodiments thereof (e.g., pharmaceutical composition comprising an anti-C5 antibody, or the functional fragment thereof, and an anti-CD40 antibody, or the functional fragment thereof in combination with at least a pharmaceutical acceptable excipient, carrier or diluent) for use in the prevention of graft rejection in a subject receiving a xenograft organ. In particular, the fourth aspect of the invention relates to a pharmaceutical composition for use in the prevention of graft rejection in a subject receiving a xenograft organ, wherein the anti-CD40 antibody is an anti-CD40 antibody, or a functional fragment thereof, with silenced ADCC activity that binds both the xenograft organ and the human CD40 and said binding inhibits CD40L induced signaling with no or low agonist activity with respect to CD40 signaling.

In a first embodiment of the fourth aspect of the invention, the antibodies are co-administered using a therapeutic composition comprising a fixed combination of the antibodies.

In a second embodiment of the fourth aspect of the invention, the pharmaceutical composition, is administered as fixed combination, wherein a) a loading dose of the anti-C5-antibody or a functional fragment thereof is administered at a dose of about 10 mg/kg to about 50 mg/kg per antibody and

b) a loading dose of the anti-CD40 antibody is administered at a dose of about 10 mg/kg to about 50 mg/kg per antibody. In an alternative second embodiment of the fourth aspect of the invention the loading dose of the anti-C5-antibody or a functional fragment thereof is administered as a single dose of about 10 mg/kg on the day of xenograft organ and the anti-CD40-antibody or a functional fragment thereof is administered as a single dose of about 10 mg/kg on the day of xenograft organ.

In a third embodiment of the fourth aspect of the invention, alone or in combination with other embodiments of the fourth aspect, the route of administration of the pharmaceutical composition is subcutaneous or intravenous.

In a fourth embodiment of the fourth aspect of the invention, alone or in combination with other embodiments of the fourth aspect, the xenograft organ is from a pig and the anti-CD40 antibody binds the pig CD40.

In an alternative fourth embodiment of the fourth aspect of the invention, the anti-CD40 antibody is an anti-CD40 antibody or functional fragment thereof with silenced ADCC activity that binds both the xenograft organ and the human CD40, wherein
d. the anti-CD40 antibody binds an epitope of the pig CD40 protein and the human CD40 protein, said epitope being comprised between amino acids 64-120 of SEQ ID NO:37 (human CD40 protein sequence, or the equivalent region in the pig CD40 protein); or
e. the anti-CD40 antibody binds to amino acids in the epitope region comprising amino acids (i) 60-70, (ii) 75-95 and (iii) 115-125 of SEQ ID NO:37 (or the equivalent region in the pig CD40 protein), or
f. the anti-CD40 antibody binds to an epitope consisting the amino acids (i) 64-75, (ii) 79-80, (iii) 82-87, (iv) 94 and (v) 118.-120 of SEQ ID NO:37 (or the equivalent region in the pig CD40 protein) and said binding inhibits CD40L induced signaling with no or low agonist activity with respect to CD40 signaling.

In an alternative fourth embodiment of the fourth aspect of the invention the pig is a transgenic organism.

In a fifth embodiment of the fourth aspect of the invention, alone or in combination with other embodiments of the fourth aspect, the transgenic donor pig has been genetically modified as follows: disrupted a(1,3)-galactosyltransferase and CMAH genes.

In an alternative fifth embodiment of the fourth aspect of the invention, the transgenic donor pig comprises disrupted a(1,3)-galactosyltransferase and CMAH genes and additional genetic modifications.

In a sixth embodiment of the fourth aspect of the invention, alone or in combination with other embodiments of the fourth aspect, the subject receiving the above disclosed pharmaceutical composition comprising an anti-CD40 antibody and an anti-C5 antibody, or functional fragments thereof, for use in the prevention of graft rejection is administered an induction therapy prior to receiving the xenotransplant. In an alternative sixth embodiment of the fourth aspect of the invention the induction therapy is administration of an anti-CD4 antibody and/or an anti-CD20 antibody. In another alternative sixth embodiment of the fourth aspect of the invention the anti-CD4 antibody and/or an anti-CD20 antibody treatment is combined with other anti-proliferative agents like mycophenolate mofetil (MMF), or steroids, like prednisone or T cell immunosuppressive compounds, like calcineurin inhibitors such as cyclosporin and tacrolimus.

In a fifth aspect, the invention relates to a combination of an anti-C5 antibody, or a functional fragment thereof, and an anti-CD40 antibody, or a functional fragment thereof, for use in the prevention of graft rejection in a subject receiving a xenotransplant.

In a first embodiment of the fifth aspect of the invention, the anti-CD40 antibody is an anti-CD40 antibody or functional fragment thereof with silenced ADCC activity that binds both the xenograft organ and the human CD40 and said binding inhibits CD40L induced signaling with no or low agonist activity with respect to CD40 signaling.

In an alternative first embodiment of the fifth aspect of the invention, the anti-CD40 antibody is an anti-CD40 antibody or functional fragment thereof with silenced ADCC activity that binds both the xenograft organ and the human CD40, wherein
a. the anti-CD40 antibody binds an epitope of the xenograft CD40 protein and the human CD40 protein, said epitope being comprised between amino acids 64-120 of SEQ ID NO:37 (human CD40 protein sequence, or the equivalent region in the xenograft CD40 protein); or
b. the anti-CD40 antibody binds to amino acids in the epitope region comprising amino acids (i) 60-70, (ii) 75-95 and (iii) 115-125 of SEQ ID NO:37 (or the equivalent region in the xenograft CD40 protein), or
c. the anti-CD40 antibody binds to an epitope consisting the amino acids (i) 64-75, (ii) 79-80, (iii) 82-87, (iv) 94 and (v) 118.-120 of SEQ ID NO:37 (or the equivalent region in the xenograft CD40 protein) and said binding inhibits CD40L induced signaling with no or low agonist activity with respect to CD40 signaling.

In a second embodiment of the fifth aspect of the invention, the combination of an anti-C5 antibody, or a functional fragment thereof, and an anti-CD40 antibody, or a functional fragment thereof, for use in the prevention of graft rejection in a subject receiving a xenotransplant is co-administered using a fixed combination of the antibodies, or both antibodies are administered in parallel or sequentially using two different pharmaceutical compositions comprising each only one of the two antibodies. In an alternative second embodiment of the fifth aspect of the invention, the antibodies are administered through a loading dose and/or a maintenance dose.

In a third embodiment of the fifth aspect of the invention, alone or in combination with other embodiments of the fifth aspect, the combination of an anti-C5 antibody, or a functional fragment thereof, and an anti-CD40 antibody, or a functional fragment thereof, for use in the prevention of graft rejection in a subject receiving a xenotransplant is administered as fixed combination, in parallel or sequentially, wherein a) the loading dose of the anti-C5-antibody is administered at a dose of about 10 mg/kg to about 50 mg/kg per antibody and b) the loading dose of the anti-CD40 antibody is administered at a dose of about 10 mg/kg to about 50 mg/kg per antibody.

In an alternative third embodiment of the fifth aspect of the invention, the loading dose of the anti-CS-antibody is administered as a single dose of about 10 mg/kg on the day of xenograft organ and the anti-CD40-antibody is administered as a single dose of about 10 mg/kg on the day of xenograft organ.

In a fourth embodiment of the fifth aspect of the invention, alone or in combination with other embodiments of the fifth aspect, the route of administration of the anti-C5 antibody or a functional fragment thereof is subcutaneous or intravenous, and/or wherein the administration the anti-CD40 antibody or a functional fragment thereof is subcutaneous or intravenous.

In a fifth embodiment of the fifth aspect of the invention, alone or in combination with other embodiments of the fifth aspect, the combination of an anti-C5 antibody, or a functional fragment thereof, and an anti-CD40 antibody, or a functional fragment thereof, for use in the prevention of graft rejection in a subject receiving a xenotransplant is used in combination with a pig organ and the anti-CD40 antibody binds the pig CD40. In an alternative fifth embodiment of the fifth aspect of the invention, the pig is a transgenic organism. In another alternative fifth embodiment of the fifth aspect of the invention, the transgenic donor pig has been genetically modified as follows: disrupted a(1,3)-galactosyltransferase and CMAH genes. In another alternative fifth embodiment of the fifth aspect of the invention, the transgenic donor pig comprises disrupted a(1,3)-galactosyltransferase and CMAH genes and additional genetic modifications.

In a sixth embodiment of the fifth aspect of the invention, alone or in combination with other embodiments of the fifth aspect, the combination of an anti-C5 antibody, or a functional fragment thereof, and an anti-CD40 antibody, or a functional fragment thereof, for use in the prevention of graft rejection in a subject receiving a xenotransplant is used in combination with an induction therapy administered to the subject prior to receiving the xenotransplant.

In an alternative sixth embodiment of the fifth aspect of the invention, the induction therapy is administration of an anti-CD4 antibody and/or an anti-CD20 antibody.

In a seventh embodiment of the fifth aspect of the invention, alone or in combination with other embodiments of the fifth aspect, the anti-CD40 antibody used in the combination with an anti-C5 antibody, or a functional fragment thereof, comprises an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30.

In an alternative seventh embodiment of the fifth aspect of the invention, alone or in combination with other embodiments of the fifth aspect, the anti-CD40 antibody used in the combination with an anti-C5 antibody, or a functional fragment thereof, comprises an immunoglobulin VH domain comprising the amino acid sequence of SEQ ID NO: 31 and an immunoglobulin VL domain comprising the amino acid sequence of SEQ ID NO: 32.

In another alternative seventh embodiment of the fifth aspect of the invention, alone or in combination with other embodiments of the fifth aspect, the anti-CD40 antibody used in the combination with an anti-C5 antibody, or a functional fragment thereof, comprises an immunoglobulin HC domain comprising the amino acid sequence of SEQ ID NO: 33 and an immunoglobulin LC domain comprising the amino acid sequence of SEQ ID NO: 34.

In an alternative seventh embodiment of the fifth aspect of the invention, alone or in combination with other embodiments of the first aspect, the anti-CD40 antibody used in the combination with an anti-C5 antibody, or a functional fragment thereof, is iscalimab.

In another different alternative seventh embodiment of the fifth aspect of the invention, alone or in combination with other embodiments of the fifth aspect, the anti-CD40 antibody used in the combination with an anti-C5 antibody, or a functional fragment thereof, comprises an immunoglobulin HC domain comprising the amino acid sequence of SEQ ID NO: 35 and an immunoglobulin LC domain comprising the amino acid sequence of SEQ ID NO: 36.

In an eighth embodiment of the fifth aspect of the invention, alone or in combination with other embodiments of the fifth aspect, the anti-C5 antibody or a functional fragment thereof used in the combination with an anti-CD40 antibody, or a functional fragment thereof, comprises an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6.

In an eighth embodiment of the fifth aspect of the invention, alone or in combination with other embodiments of the fifth aspect, the anti-C5 antibody or a functional fragment thereof used in the combination with an anti-CD40 antibody, or a functional fragment thereof, comprises an immunoglobulin VH domain comprising the amino acid sequence of SEQ ID NO: 7 and an immunoglobulin VL domain comprising the amino acid sequence of SEQ ID NO: 8.

In a ninth embodiment of the fifth aspect of the invention, alone or in combination with other embodiments of the fifth aspect, the anti-C5 antibody or a functional fragment thereof used in the combination with an anti-CD40 antibody, or a functional fragment thereof, comprises an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16.

In a tenth embodiment of the fifth aspect of the invention, alone or in combination with other embodiments of the fifth aspect, the anti-C5 antibody used in the combination with an anti-CD40 antibody, or a functional fragment thereof, comprises an immunoglobulin VH domain comprising the amino acid sequence of SEQ ID NO: 17 and an immunoglobulin VL domain comprising the amino acid sequence of SEQ ID NO: 18.

In an eleventh embodiment of the fifth aspect of the invention, alone or in combination with other embodiments of the fifth aspect, the anti-C5 antibody used in the combination with an anti-CD40 antibody, or a functional fragment thereof, is tesidolumab or eculizumab.

In a sixth aspect, the invention relates to a method of suppressing the rejection and prolonging the survival of a xenograft donor organ from an animal in a human recipient, the method comprising administering to a human recipient an anti-C5 antibody and an anti-CD40 antibody or functional fragments thereof.

In a first embodiment of the sixth aspect of the invention the method of suppressing the rejection and/or prolonging the survival of a xenograft organ from an animal in a human recipient comprises the use of a pharmaceutical composition according to the third aspect of the invention and all disclosed embodiments thereof (e.g. pharmaceutical composition comprising an anti-C5 antibody, or the functional fragment thereof, and an anti-CD40 antibody, or the functional fragment thereof, in combination with at least a pharmaceutical acceptable excipient, carrier or diluent). In an alternative first embodiment of the sixth aspect of the invention the method of suppressing the rejection and prolonging the survival of a xenograft organ from an animal in a human recipient comprises the use of a combination according to fifth aspect of the invention and all embodiments thereof (e.g. a combination of an anti-C5 antibody, or a functional fragment thereof, and an anti-CD40 antibody, or a functional fragment thereof, for use in the prevention of graft rejection in a subject receiving a xenotransplant).

In a second embodiment of the sixth aspect of the invention, alone or in combination with other embodiments of the sixth aspect, the anti-C5 antibody, or the functional fragment thereof, and the anti-CD40 antibody, or the functional fragment thereof, are co-administered using a fixed combination of the antibodies, or both antibodies are administered in parallel or sequentially using two different pharmaceutical compositions comprising each only one of the two antibodies through a loading dose and/or a maintenance dose.

In a third embodiment of the sixth aspect of the invention, alone or in combination with other embodiments of the sixth aspect, the method of suppressing the rejection and prolonging the survival of a xenograft organ from an animal in a human recipient comprises administration of the anti-C5 antibody, or the functional fragment thereof, and the anti-CD40 antibody, or the functional fragment thereof, as fixed combination, wherein a) a loading dose of the anti-C5-antibody or a functional fragment thereof is administered at a dose of about 10 mg/kg to about 50 mg/kg per antibody and b) a loading dose of the anti-CD40 antibody or a functional fragment thereof is administered at a dose of about 10 mg/kg to about 50 mg/kg per antibody. In an alternative third embodiment of the sixth aspect of the invention, alone or in combination with other embodiments of the sixth aspect, the loading dose of the anti-CS-antibody or a functional fragment thereof is administered as a single dose of about 10 mg/kg on the day of xenograft organ and the anti-CD40-antibody or a functional fragment thereof is administered as a single dose of about 10 mg/kg on the day of xenograft organ.

In a fourth embodiment of the sixth aspect of the invention, alone or in combination with other embodiments of the sixth aspect, the route of administration of the anti-C5 antibody, or the functional fragment thereof, and the anti-CD40 antibody, or the functional fragment thereof is subcutaneous or intravenous.

In a fifth embodiment of the sixth aspect of the invention, alone or in combination with other embodiments of the sixth aspect, the xenograft organ is from a pig and the anti-CD40 antibody or a functional fragment thereof binds the pig CD40.

In an alternative fifth embodiment of the sixth aspect of the invention, the anti-CD40 antibody is an anti-CD40 antibody or functional fragment thereof with silenced ADCC activity that binds both the xenograft organ and the human CD40, wherein
a. the anti-CD40 antibody binds an epitope of the xenograft CD40 protein and the human CD40 protein, said epitope being comprised between amino acids 64-120 of SEQ ID NO:37 (human CD40 protein sequence, or the equivalent region in the xenograft CD40 protein); or
b. the anti-CD40 antibody binds to amino acids in the epitope region comprising amino acids (i) 60-70, (ii) 75-95 and (iii) 115-125 of SEQ ID NO:37 (or the equivalent region in the xenograft CD40 protein), or
c. the anti-CD40 antibody binds to an epitope consisting the amino acids (i) 64-75, (ii) 79-80, (iii) 82-87, (iv) 94 and (v) 118.-120 of SEQ ID NO:37 (or the equivalent region in the xenograft CD40 protein) and said binding inhibits CD40L induced signaling with no or low agonist activity with respect to CD40 signaling.

In an alternative fifth embodiment of the sixth aspect of the invention, alone or in combination with other embodiments of the sixth aspect, the pig is a transgenic organism. In another alternative fifth embodiment of the sixth aspect of the invention, alone or in combination with other embodiments of the sixth aspect, the transgenic donor pig has been genetically modified as follows: disrupted a(1,3)-galactosyltransferase and CMAH genes.

In an alternative sixth embodiment of the sixth aspect of the invention, the transgenic donor pig comprises disrupted a(1,3)-galactosyltransferase and CMAH genes and additional genetic modifications (e.g. as described below).

In a sixth embodiment of the sixth aspect of the invention, alone or in combination with other embodiments of the sixth aspect, the method of suppressing the rejection and prolonging the survival of a xenograft organ from an animal in a human recipient comprises an induction therapy administered to the subject prior to receiving the xenotransplant. In an alternative sixth embodiment of the sixth aspect of the invention, alone or in combination with other embodiments of the sixth aspect, the induction therapy is administration of an anti-CD4 antibody and/or an anti-CD20 antibody.

In a seventh embodiment of the sixth aspect of the invention, alone or in combination with other embodiments of the sixth aspect, the method of suppressing the rejection and prolonging the survival of a xenograft organ from an animal in a human recipient comprises using an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30.

In an eight embodiment of the sixth aspect of the invention, alone or in combination with other embodiments of the sixth aspect, the anti-CD40 antibody comprises an immunoglobulin VH domain comprising the amino acid sequence of SEQ ID NO: 31 and an immunoglobulin VL domain comprising the amino acid sequence of SEQ ID NO: 32.

In a ninth embodiment of the sixth aspect of the invention, alone or in combination with other embodiments of the sixth aspect, the anti-CD40 antibody comprises an immunoglobulin HC domain comprising the amino acid sequence of SEQ ID NO: 33 and an immunoglobulin LC domain comprising the amino acid sequence of SEQ ID NO: 34.

In an alternative tenth embodiment of the sixth aspect of the invention, alone or in combination with other embodiments of the sixth aspect, the anti-CD40 antibody is iscalimab.

In a eleventh embodiment of the sixth aspect of the invention, alone or in combination with other embodiments of the sixth aspect, the anti-CD40 antibody comprises an immunoglobulin HC domain comprising the amino acid sequence of SEQ ID NO: 35 and an immunoglobulin LC domain comprising the amino acid sequence of SEQ ID NO: 36.

In a twelfth embodiment of the sixth aspect of the invention, alone or in combination with other embodiments of the sixth aspect, the method of suppressing the rejection and prolonging the survival of a xenograft organ from an animal in a human recipient comprises using an anti-C5 antibody or a functional fragment thereof comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6.

In a thirteenth embodiment of the sixth aspect of the invention, alone or in combination with other embodiments of the sixth aspect, the anti-C5 antibody or a functional fragment thereof comprised in the pharmaceutical composition is an anti-C5 antibody comprising an immunoglobulin VH domain comprising the amino acid sequence of SEQ ID NO: 7 and an immunoglobulin VL domain comprising the amino acid sequence of SEQ ID NO: 8.

In a fourteenth embodiment of the sixth aspect of the invention, alone or in combination with other embodiments of the sixth aspect, the anti-C5 antibody or a functional fragment thereof comprised in the pharmaceutical composition is an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16.

In a fifteenth embodiment of the sixth aspect of the invention, alone or in combination with other embodiments of the sixth aspect, the anti-C5 antibody or a functional fragment thereof comprised in the pharmaceutical composition is an anti-C5 antibody comprising an immunoglobulin VH domain comprising the amino acid sequence of SEQ ID NO: 17 and an immunoglobulin VL domain comprising the amino acid sequence of SEQ ID NO: 18.

In a sixteenth embodiment of the sixth aspect of the invention, alone or in combination with other embodiments of the sixth aspect, the anti-C5 antibody is tesidolumab or eculizumab.

In a seventeenth embodiment of the sixth aspect of the invention, alone or in combination with other embodiments of the sixth aspect, the method of suppressing the rejection and prolonging the survival of a xenograft organ from an animal in a human recipient is combined with other anti-proliferative agents like mycophenolate mofetil (MMF), or steroids, like prednisone or T cell immunosuppressive compounds, like calcineurin inhibitors such as cyclosporin and tacrolimus.

In a seventh aspect, the invention relates to the use of an anti-C5 antibody and an anti-CD40 antibody, or functional fragments thereof, in the manufacture of a medicament for the prevention of graft rejection in a subject receiving a xenograft.

In a first embodiment of the seventh aspect of the invention, alone or in combination with other embodiments of the seventh aspect, the anti-CD40 antibody is an anti-CD40 antibody or functional fragment thereof with silenced ADCC activity that binds both the xenograft organ and the human CD40 and said binding inhibits CD40L induced signaling with no or low agonist activity with respect to CD40 signaling.

In a second embodiment of the seventh aspect of the invention, alone or in combination with other embodiments of the seventh aspect, the anti-CD40 antibody or a functional fragment thereof used in the manufacture of a medicament for the prevention of graft rejection in a subject receiving a xenograft comprises an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30.

In a third embodiment of the seventh aspect of the invention, alone or in combination with other embodiments of the seventh aspect, the anti-CD40 antibody or a functional fragment thereof comprises an immunoglobulin VH domain comprising the amino acid sequence of SEQ ID NO: 31 and an immunoglobulin VL domain comprising the amino acid sequence of SEQ ID NO: 32.

In a fourth embodiment of the seventh aspect of the invention, alone or in combination with other embodiments of the seventh aspect, the anti-CD40 antibody or a functional fragment thereof comprises an immunoglobulin HC domain comprising the amino acid sequence of SEQ ID NO: 33 and an immunoglobulin LC domain comprising the amino acid sequence of SEQ ID NO: 34.

In an alternative fourth embodiment of the seventh aspect of the invention, alone or in combination with other embodiments of the seventh aspect, the anti-CD40 antibody is iscalimab.

In a fifth embodiment of the seventh aspect of the invention, alone or in combination with other embodiments of the seventh aspect, the anti-CD40 antibody comprises an immunoglobulin HC domain comprising the amino acid sequence of SEQ ID NO: 35 and an immunoglobulin LC domain comprising the amino acid sequence of SEQ ID NO: 36.

In a sixth embodiment of the seventh aspect of the invention, the anti-C5 antibody or a functional fragment thereof used in the manufacture of a medicament for the prevention of graft rejection in a subject receiving a xenograft comprises an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6.

In a seventh embodiment of the seventh aspect of the invention, alone or in combination with other embodiments of the seventh aspect, the anti-C5 antibody or a functional fragment thereof used in the manufacture of a medicament for the prevention of graft rejection in a subject receiving a xenograft comprises immunoglobulin VH domain comprising the amino acid sequence of SEQ ID NO: 7 and an immunoglobulin VL domain comprising the amino acid sequence of SEQ ID NO: 8.

In an eighth embodiment of the seventh aspect of the invention, alone or in combination with other embodiments of the seventh aspect, the anti-C5 antibody or a functional fragment thereof used in the manufacture of a medicament for the prevention of graft rejection in a subject receiving a xenograft comprises immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16.

In a ninth embodiment of the seventh aspect of the invention, alone or in combination with other embodiments of the seventh aspect, the anti-C5 antibody or a functional fragment thereof used in the manufacture of a medicament for the prevention of graft rejection in a subject receiving a xenograft comprises immunoglobulin VH domain comprising the amino acid sequence of SEQ ID NO: 17 and an immunoglobulin VL domain comprising the amino acid sequence of SEQ ID NO: 18.

In a tenth embodiment of the seventh aspect of the invention, alone or in combination with other embodiments of the seventh aspect, the anti-C5 antibody is tesidolumab or eculizumab.

In an eighth aspect, the invention relates to a kit of parts comprising: (i) an anti-CD40 antibody, or a functional fragment thereof, with silenced ADCC activity that binds both the xenograft organ and the human CD40 and said binding inhibits CD40L induced signalling with no or low agonist activity with respect to CD40 signaling, (ii) an anti-C5 antibody, (iii) administration means, (iv) instructions for their use and optionally further comprising (v) at least one other excipients, diluents or carriers.

In a first embodiment of the eighth aspect of the invention, the anti-CD40 antibody comprised in the kit of parts is an anti-CD40 antibody, or a functional fragment thereof, comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30. In a second embodiment of the eighth aspect of the invention, alone or in combination with other embodiments of the eighth aspect, the anti-CD40 antibody or a functional fragment thereof comprises an immunoglobulin VH domain comprising the amino acid sequence of SEQ ID NO: 31 and an immunoglobulin VL domain comprising the amino acid sequence of SEQ ID NO: 32.

In a third embodiment of the eighth aspect of the invention, alone or in combination with other embodiments of the eighth aspect, the anti-CD40 antibody comprises an immunoglobulin HC domain comprising the amino acid sequence of SEQ ID NO: 33 and an immunoglobulin LC domain comprising the amino acid sequence of SEQ ID NO: 34.

In an alternative third embodiment of the eighth aspect of the invention, alone or in combination with other embodiments of the eighth aspect, the anti-CD40 antibody is iscalimab.

In a fourth embodiment of the eighth aspect of the invention, alone or in combination with other embodiments of the eighth aspect, the anti-CD40 antibody comprises an immunoglobulin HC domain comprising the amino acid sequence of SEQ ID NO: 35 and an immunoglobulin LC domain comprising the amino acid sequence of SEQ ID NO: 36.

In a fifth embodiment of the eighth aspect of the invention, alone or in combination with other embodiments of the eighth aspect, the anti-C5 antibody comprised in the kit of parts is an anti-C5 antibody, or a functional fragment thereof, comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6.

In a sixth embodiment of the eighth aspect of the invention, alone or in combination with other embodiments of the eighth aspect, the anti-C5 antibody comprised in the kit of parts is an anti-C5 antibody, or a functional fragment thereof, comprising an immunoglobulin VH domain comprising the amino acid sequence of SEQ ID NO: 7 and an immunoglobulin VL domain comprising the amino acid sequence of SEQ ID NO: 8.

In a seventh embodiment of the eighth aspect of the invention, alone or in combination with other embodiments of the eighth aspect, the anti-C5 antibody comprised in the kit of parts is an anti-C5 antibody, or a functional fragment thereof, comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16.

In an eighth embodiment of the eighth aspect of the invention, alone or in combination with other embodiments of the eighth aspect, the anti-C5 antibody comprised in the kit of parts is an anti-C5 antibody, or a functional fragment thereof, comprises an immunoglobulin VH domain comprising the amino acid sequence of SEQ ID NO: 17 and an immunoglobulin VL domain comprising the amino acid sequence of SEQ ID NO: 18. In a ninth embodiment of the eighth aspect of the invention, alone or in combination with other embodiments of the eighth aspect, the anti-C5 antibody is tesidolumab or eculizumab.

In a tenth embodiment of the eighth aspect of the invention, alone or in combination with other embodiments of the eighth aspect, the kit of parts comprises the pharmaceutical composition of the third aspect of the invention (e.g. comprising an anti-C5 antibody, or the functional fragment thereof, and an anti-CD40 antibody, or the functional fragment thereof in combination with at least a pharmaceutical acceptable excipient, carrier or diluents)

### BRIEF DESCRIPTION OF THE DRAWINGS

FIG. 1 (CFZ533 and CD21 double stain—FACS sorting) shows that CFZ533 is able to bind to pig PBMC. Limited or no binding observed with an anti-human CD40 FACS antibody and anti-pig CD20 FACS antibody.

FIG. 2 shows that the pig PBMCs can be stimulated to proliferate (measured using tritiated thymidine) by human rCD154 (CD40L). PMA/ionomycin is used as a positive control. FIG. 3 shows flow cytometric analyses of pig PBMCs stained for T cells (CD3) and B cells (CD21) from three different donors.

FIG. 4 shows that the cell expressing CD21 (B-cells) bind CFZ533 whereas T cells (CD3+) are not bound by CFZ533. Results using PBMCs from three different pigs are shown.

FIG. 5 shows another way of presenting the data of the FIG. 4 showing that CFZ533 binds all cells expressing CD21 (B cells). Results using PBMCs from three different pigs are shown.

FIG. 6 shows that proliferation of pig PBMCs induced by recombinant hCD40L can be inhibited by CFZ533; functional proof that CFZ533 binds and blocks activation downstream of pig CD40. Results using PBMCs from three different pigs are shown. MPG22951, MPG22949, MPG22950: Lot# of the PBMC vials; each lot being a different donor.

FIG. 7 shows binding affinity of the CD40 prior art antibody 2C10 and CFZ533 to non-human-primate (NHP) cells.

FIG. 8 shows the treatment regimen used in the disclosed NHP xeno organ transplant experiments using anti CD-40 and anti-C5 antibodies.

FIGS. 9A) and B) show the results of the NHP xeno organ transplant experiments described herein and disclosed in detail in FIG. 8.

FIGS. 10A) and B) show the Serum creatinine levels and urine protein levels in NHP having received the xeno organ as disclosed herein and treated as described in FIG. 8.

FIGS. 11A) and B) show tissue sections of NHP having received the xeno organ as disclosed herein and treated as described in FIG. 8.

FIG. 12 shows an amino acid similarity plot of CD40 proteins from different organisms.

FIG. 13 shows an amino acid sequence alignment of CD40 proteins from different organisms. Figure discloses SEQ ID NOS 38, 37 and 39, respectively, in order of appearance.

### DEFINITIONS

“A binding that inhibits CD40L induced signalling with no or low agonist activity with respect to CD40 signaling” according to the disclosure refers to an antibody that inhibits CD40L induced signaling and exhibits no or low agonist activity, as measured in a CD40L-mediated PBMC proliferation assay known to the person skilled in the art, wherein said antibody or protein inhibits CD40L induced signalling with an IC50 of 50 ng/ml or less. A CD40 antibody that inhibits CD40L induced signalling with no or low agonist activity with respect to CD40 signaling refers to an antagonistic antibody or protein that inhibits CD40 induced signaling activity in the presence of CD40L in a human cell assay such as the CD40L-mediated PBMC proliferation assay by at least 50% or 60% or 70% or 80% or 90% or 95% or more. Such assay is described in more detail in the examples below. The above referenced CD40L-mediated PBMC proliferation assay has been disclosed in detail in the method and example section of the patent application WO2012/065950. The method and example section of WO2012/065950, in particularly the methods disclosed in 1-7 of the method section (starting at page 46) as well as Example 1 (pages 57/58), are herein cooperated by reference.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

As used herein, the term “ADCC” or “antibody-dependent cellular cytotoxicity” activity refers to cell depleting activity. ADCC activity can be measured by the ADCC assay as well known to a person skilled in the art. For example, ADCC assays are described in detail in the example section of the patent application WO2012065950 e.g., example 3 (ADCC assay, page 48) of the methods section, which is herewith incorporated by reference.

In one embodiment, the term “no or low ADCC activity” means that the silent antibody exhibits an ADCC activity that is below 50% specific cell lysis, for example below 10% specific cell lysis as measured in a standard ADCC assay. No ADCC activity means that the silent antibody exhibits an ADCC activity (specific cell lysis) that is below 1%.

As used herein, the term “administration” or “administering” means providing a therapeutic agent of the invention and prodrugs thereof to a subject in need of treatment. The term “administering” encompasses administration of an anti-CD40 antibody or antigen binding/functional fragment thereof, e.g., iscalimab or an antigen binding/functional fragment thereof and/or an anti-C5 antibody or antigen binding/functional fragment thereof, e.g., tesidolumab or an antigen binding/functional fragment thereof, in a single or multiple intravenous or subcutaneous doses.

Administration “in combination with” one or more further therapeutic agents includes simultaneous (concurrent) and consecutive administration in any order, and in any route of administration.

As used herein, the term “affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a K_{D} of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a Ko of 10⁻⁷ M or less, or 10⁻⁸ M or less.

The term “antibody” as used herein refers to whole antibodies that interact with (e.g., by binding, steric hindrance, stabilizing/destabilizing, spatial distribution) an antigen. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed hypervariable regions or complementarity determining regions (CDRs), interspersed with regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelid antibodies, or chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass, preferably IgG and most preferably IgG1. Exemplary antibodies include tesidolumab (LFG316) and iscalimab (CFZ533), having the amino acid sequences as set forth in Table 1. Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The term “functional fragment” of an antibody is used herein interchangeably and refer to full length or one or more fragments of an antibody, such as a protein, that retain the ability to specifically bind to an antigen or epitope (e.g., C5 or CD40). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883).

“Binds both the xenograft organ and the human CD40” according to the disclosure refers to an anti-CD40 antibody or functional fragment thereof having the ability to bind the human CD40 polypeptide (as defined further below) and the CD40 polypeptide of the xenograft (e.g. the CD40 of a pig xenograft organ, like a pig kidney), wherein said binding of the anti-CD40 antibody inhibits CD40L induced signalling via the xenograft as well as human CD40 as measured in a PBMC proliferation assay by at least 50% or 60% or 70% or 80% or 90% or 95% or more.

As used herein, “CD40” refers to cluster of differentiation 40, also called tumor necrosis factor receptor superfamily member 5. The term CD40 refers to human CD40, for example as defined in SEQ ID NO: 37, unless otherwise described.

As used herein, “combination” refers to either a fixed combination in one dosage unit form, or a combined administration where an anti-CD40 antibody (or a functional fragment thereof) and an anti-C5 antibody (or a functional fragment thereof) may be administered independently at the same time or separately within time intervals, especially where these time intervals allow that the combination partners show a therapeutic or cooperative, e.g., synergistic effect. The single anti-CD40 antibody (or a functional fragment thereof) and an anti-C5 antibody (or a functional fragment thereof) may be packaged in a kit or separately. One or both of the anti-CD40 antibody (or a functional fragment thereof) and the anti-C5 antibody (or a functional fragment thereof) (e.g., provided as powders or liquids) may be reconstituted or diluted to a desired dose prior to administration. The terms “co-administration” or “combined administration” or the like as utilized herein are meant to encompass administration of the selected anti-CD40 antibody (or a functional fragment thereof) and an anti-C5 antibody (or a functional fragment thereof) to a single subject in need thereof (e.g., a patient), and are intended to include treatment regimens in which the agents are not necessarily administered by the same route of administration or at the same time. The term “fixed combination” means that the therapeutic agents, e.g., an anti-CD40 antibody (or a functional fragment thereof) and an anti-C5 antibody (or a functional fragment thereof), are both administered to a patient simultaneously in the form of a single entity or dosage. The term “non-fixed combination” means that the therapeutic agents, e.g., an anti-CD40 antibody (or a functional fragment thereof) and an anti-C5 antibody (or a functional fragment thereof), are both administered to a patient as separate entities either simultaneously, concurrently or sequentially with no specific time limits, wherein such administration provides therapeutically effective levels of the two antibodies in the body of the patient.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single injection having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The “Complementarity Determining Regions”(“CDRs”) are amino acid sequences with boundaries determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). Under IMGT, the CDR regions of an antibody can be determined using the program IMGT/DomainGap Align.

The term “comprising” encompasses “including” as well as “consisting,” e.g., a composition “comprising” X may consist exclusively of X or may include something additional, e.g., X+Y.

An antibody that “cross-reacts” with an antigen other than C5 (in particular C5α) or CD40 is intended to refer to an antibody that binds that antigen with a K_{D} of 1 μM or less, 100 nM or less, 10 nM or less, 1 nM or less. An antibody that does not cross-react with a particular antigen is intended to refer to an antibody that binds to that antigen, with a K_{D} of 100 nM or greater, or a K_{D} of 1 μM or greater, or a K_{D} of 10 μM or greater. In certain embodiments, such antibodies that do not cross-react with the antigen exhibit essentially undetectable binding against these proteins in standard binding assays.

The term “epitope” as used herein refers to any determinant capable of binding with high affinity to an immunoglobulin. An epitope is a region of an antigen that is bound by an antibody that specifically targets that antigen, and when the antigen is a protein, includes specific amino acids that directly contact the antibody. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three-dimensional structural characteristics, and/or specific charge characteristics. Conformational and non-conformational epitopes are distinguished in that the binding to the former but not the latter is lost in the presence of denaturing solvents. Epitope mapping technologies are well known in the art.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. Such a constant region is modified compared to a wild-type constant region. That is, the polypeptides used in the invention compositions, uses or methods disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life and silencing, etc.

The terms “individual”, “host”, “subject”, and “patient” are used interchangeably to refer to the subject, for example, a non-human primate or human patient, that is the object of treatment, observation and/or experiment. According to the invention, the subject can be an organ transplant patient, e.g., a xenotransplant organ recipient, or can be a patient waiting for a xenoorgan transplantation. For example, the subject is a xenokidney transplant or a xenokidney transplant candidate.

As used herein, a subject is “in need of” a treatment if such subject would benefit biologically, medically or in quality of life from such treatment.

The phrase “induction therapy” or “induction regimen” refers to a treatment regimen (or the portion of a treatment regimen) that is used for the initial treatment of a condition. In some embodiments, the disclosed methods, uses, kits, processes and regimens (e.g., methods of preventing graft loss in xenotransplantation) employ an induction regimen. In some cases, the induction period is the period until maximum efficacy is reached. The general goal of an induction regimen is to provide a high level of drug to a subject during the initial period of a treatment regimen. An induction regimen may employ (in part or in whole) a “loading dose regimen” or “loading dose”, which may include administering a greater dose of the therapeutic agent(s) than a physician would employ during a maintenance regimen, administering a therapeutic agent(s) more frequently than a physician would administer the therapeutic agent(s) during a maintenance regimen, or both. Dose escalation may occur during or after an induction regimen.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction.

The term “KD”, as used herein, is intended to refer to the dissociation constant, which is obtained from the ratio of Kd to Ka (i.e. Kd/Ka) and is expressed as a molar concentration (M). KD values for antibodies can be determined using methods well established in the art. A method for determining the KD of an antibody is by using surface plasmon resonance, for example, by using a biosensor system such as Biacore®.

The “loading dose” may be defined as a dose higher than a maintenance dose. As herein defined, the loading phase is the period at the beginning of treatment during which the dose of therapeutic agent(s) that is administered to the subject, is higher than the maintenance dose of the therapeutic agent(s). The loading phase is optional. It can last for at least one week, one week, two weeks or one month. It can start before xenotransplantation, on the day of xenotransplantation or after xenotransplantation, e.g., on the day of xenotransplantation.

The phrase “maintenance therapy” or “maintenance regimen” or “maintenance dose” refers to a treatment regimen (or the portion of a treatment regimen) that is used for the maintenance of a subject during treatment of an illness, e.g., to keep the subject in remission for long periods of time (months or years) following the induction period. In some embodiments, the disclosed methods, uses and regimens employ a maintenance regimen. A maintenance regimen may employ (in part or in whole) a “maintenance dose” or “maintenance dosing” or a “maintenance dosing regimen”, administered through continuous therapy (e.g., administering a drug at a regular intervals, e.g., twice a week, weekly, every two weeks, monthly [every 4 weeks], yearly, lifelong etc.) or intermittent therapy (e.g., interrupted treatment, intermittent treatment, treatment at relapse, or treatment upon achievement of a particular predetermined criteria e.g., pain, disease manifestation, etc.). Dose escalation may occur during a maintenance regimen.

The phrase “means for administering” is used to indicate any available implement for systemically administering a therapeutic agent to a subject, including, but not limited to, a pre-filled syringe, a vial and syringe, an injection pen, an autoinjector, an i.v. drip and bag, a pump, a patch pump, etc. With such items, a subject may self-administer the therapeutic agent (i.e., administer the therapeutic agent on their own behalf) or a physician may administer the therapeutic agent.

The term “pharmaceutically acceptable” means a nontoxic material that does not interfere with the effectiveness of the biological activity of the active ingredient(s).

“Plasma concentration” is the blood plasma concentration of a subject.

The term “prevent” or “prevention” refers to a complete inhibition of development of a disease, condition or disorder, but also refers to the prophylactic treatment of a subject who is at risk of developing a condition, e.g., losing a transplanted organ.

“Prevention of graft rejection” or “long-term prevention of graft rejection”, “long-term prevention of graft loss”, “long term graft survival”or “suppressing the rejection and prolonging the survival of a xenograft” or “suppressing the rejection of a xenograft organ” in transplant patients, in particular in solid organ transplant patients (including in kidney transplantation, liver transplantation, heart transplantation, lung transplantation, pancreas transplantation, intestine transplantation or composite tissue transplantation) refers to (i) a situation in which the transplanted tissue or organ or graft survives and functions for a period of at least 3 years, or at least 4 years, or at least 5 years post transplantation and (ii) a situation in which the transplanted tissue or organ or graft survives and functions for a period that is at least 6 month, or at least 1 year, or at least 2 years, or at least 3 longer compared to a situation in which the inventive compositions, uses or methods have not been applied to the subject and (iii) a situation in which the risk of graft rejection is reduced.

The term “repeated administration”or “administered repeatedly”, as used herein, refers to administration of the herein disclosed pharmaceutical compositions or therapeutic combinations, at an administration interval between two administrations of not more than one month, not more than three weeks, not more than two weeks, not more than one week, at least 3 months, at least 6 months, at least 9 months or at least 1 year.

The subject can be “sensitized” or “pre-sensitized”. The subject can be of high risk or medium risk of AMR, as hereinabove defined. In another embodiment, the subject may have previously received a transplant e.g., an allotransplant or a xenotransplant.

As used herein, the term “silent” antibody refers to an antibody that exhibits no or low ADCC activity as measured in an ADCC assay. Silenced effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181:6664-69; Strohl, W., supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation (e.g., like the antibody CD40 mAb2). Another silent IgG1 antibody comprises the N297A mutation (e.g., like CFZ533), which results in aglycosylated/non-glycosylated antibodies.

As used herein, an antibody or a protein that “specifically binds to C5α” is intended to refer to an antibody or protein that binds to the alpha chain of human complement protein C5 with a K_{D} of 100 nM or less, 10 nM or less, 1 nM or less.

As used herein, an antibody or a protein that “specifically binds to CD40” is intended to refer to an antibody or protein that binds to human CD40 polypeptide with a K_{D} of 100 nM or less, 10 nM or less, 1 nM or less.

The word “substantially” does not exclude “completely,” e.g., a composition which is “substantially free” from Y may be completely free from Y. Where necessary, the word “substantially” may be omitted from the definition of the disclosure.

As used herein, a “therapeutically effective amount” refers to an amount of an anti-C5 antibody or antigen binding/functional fragment thereof, an anti-CD40 antibody or antigen binding/functional fragment thereof and/or an immunosuppressive agent, that is effective, upon single or multiple dose administration to a subject (such as a human) for treating, preventing, preventing the onset of, curing, delaying, reducing the severity of, ameliorating at least one symptom of a disorder or recurring disorder, or prolonging the survival of the subject beyond that expected in the absence of such treatment. When applied to refer to an individual active ingredient (e.g., an anti-C5α antibody) administered alone, the term refers to that ingredient alone. When applied to a combination, the term refers to combined amounts of the active ingredients that result in the therapeutic effect, whether administered in combination, serially or simultaneously.

The phrase “therapeutic regimen” means the regimen used to treat a condition, e.g., the dosing protocol used during the prevention of graft loss in xenotransplantation. A therapeutic regimen may include an induction regimen and a maintenance regimen.

“transgenic donor organism (e.g., a pig) that has been genetically modified” according to the disclosure refers to an animal suitable to be used as a xenograft donor that has been genetically modified in order to increase the compatibility of such an organ with the human recipient immune system (e.g. in order to prevent/reduce the risk of xenograft rejection) as described herein in detail. The main issue using xenografts in human is the immune response (e.g., rejection by antibody-mediated and cell-mediated responses) and incompatibilities including uncontrolled complement activation and blood coagulation abnormalities. The main goal of genetic modification of pig xenograft organ donors is to create the least immunogenic organ possible and using the best immunosuppression to deliver it.

The term “treating” or “treatment” as used herein includes the administration to a subject of an anti-C5 antibody, an anti-CD40 antibody and/or an immunosuppressive agent according to the invention to prevent or delay the onset of the symptoms, complications, or biochemical indicia of a disease, condition or disorder (e.g., AMR), alleviating the symptoms or arresting or inhibiting further development of the disease, condition or disorder. Treatment may be prophylactic (to prevent or delay the onset of the disease, condition or disorder, or to prevent the manifestation of clinical or subclinical symptoms thereof) or therapeutic suppression or alleviation of symptoms after the manifestation of the disease, condition or disorder. Within the meaning of the present invention, the term “treat” also denotes to arrest, delay the onset (i.e., the period prior to clinical manifestation of a disease, condition, or disorder) and/or reduce the risk of developing or worsening a disease, condition or disorder.

As used herein, the terms “trough level” and “trough concentration” refer to the lowest levels of free anti-CD40 antibody or antigen binding/functional fragment thereof in a sample (e.g., a serum or plasma sample, e.g., serum) from a subject over a period of time. In certain embodiments, the period of time is the entire period of time between the administration of one dose of the anti-CD40 antibody or an antigen binding/functional fragment thereof and another dose of said antibody or antigen binding/functional fragment thereof. In some embodiments, the period of time is approximately 24 hours, approximately 48 hours, approximately 72 hours, approximately 7 days, or approximately 14 days after the administration of one dose of said antibody or antigen binding/functional fragment thereof and before the administration of another dose of said antibody or antigen binding/functional fragment thereof.

“Xenograft” according to the disclosure can include an organ, part of an organ, tissue or cell transplanted from one species to another. These include, but are not limited to, heart, kidney, lung, pancreas, liver, vascular tissue, eye, cornea, lens, skin, bone marrow, muscle, connective tissue, gastrointestinal tissue, nervous tissue, bone, stem cells, islets, cartilage, hepatocytes, and hematopoietic cells. In one embodiment, the human subject is a solid organ transplant patient, preferably a kidney transplant patient and the xeno organ is obtained from a pig. The term “solid organ”, as used herein, refers to an internal organ that has a firm tissue consistency and is neither hollow (such as the organs of the gastrointestinal tract) nor liquid (such as blood). Such organs include the heart, kidney, liver, lungs, and pancreas.

### DETAILED DESCRIPTION

Without wishing to be bound by theory, the inventors have identified that anti-CD40 monoclonal antibodies with silenced ADCC activity that bind both the xenograft CD40 and the human CD40, wherein said binding inhibits CD40L induced signaling with no or low agonist activity with respect to CD40 signaling, are suitable for the prevention of graft rejection in a subject receiving a xenograft organ. The inventors have furthermore identified that a combination of CD40 signaling inactivation with inactivation of the complement system, in particular a combination of the above described CD40 antibody or a functional fragment thereof with an inhibitor of the complements system is suitable for prevention of graft rejection in a subject receiving a xenograft organ. The inventors have also identified that a combination of an anti-CD40 monoclonal antibody with silenced ADCC activity that bind both the xenograft organ CD40 and the human CD40—as described above—with an anti-C5 antibody or a functional fragment thereof is particularly suitable for the prevention of graft rejection in a subject receiving a xenograft organ

Any C5 pathway antagonist, such as a monoclonal antibody capable of blocking formation of the membrane attack complex (MAC), e.g., an anti-C5 antibody, can be combined in the disclosed methods or treatments for the prevention of graft loss in solid organ xenotransplantation with any anti-CD40 antibody with silenced ADCC activity that bind both the xenograft organ CD40 and the human CD40—as described above. A dosing regimen providing throughout the entire treatment period plasma concentrations of (i) an anti-CD40 antibody or functional fragment thereof or (ii) an anti-CD40 antibody or functional fragment thereof and an inhibitor of the complements system, e.g., an anti C5 antibody resulting in a therapeutic effect is therefore desirous.

According to the disclosure, the anti-CD40 antibody to be administered according to the herein disclosed methods or treatments binds to CD40, a transmembrane glycoprotein constitutively expressed on B cells and antigen presenting cells (APCs) such as monocytes, macrophages, and dendritic cells (DC) having the amino acid sequence shown in SEQ ID NO: 37. CD40 is also expressed on platelets, and under certain conditions can be expressed on eosinophils, and parenchymal cells.

1. Pharmaceutical Compositions for Use in the Prevention of Xenograft Rejection/Methods for the Prevention of Xenograft Rejection Using a Pharmaceutical Composition Comprising an Anti-CD40 Antibody, or the Functional Fragment Thereof in Combination with at Least a Pharmaceutically Acceptable Excipient, Carrier or Diluent

Therefore, in one aspect, the disclosure relates to a pharmaceutical composition comprising an anti-CD40 antibody or functional fragment thereof for use in the prevention of graft rejection in a subject receiving a xenograft organ, wherein the anti-CD40 antibody is an anti-CD40 antibody, or a functional fragment thereof, (i) with silenced ADCC activity that (ii) binds both the xenograft organ and the human CD40 and (iii) said binding inhibits CD40L induced signaling with (iv) no or low agonist activity with respect to CD40 signaling.

In another aspect of the disclosure, a method of suppressing the rejection and prolonging the survival of a xenograft donor organ from an animal in a human recipient is provided, said method comprises administering to the human recipient an anti-CD40 antibody, wherein the anti-CD40 antibody is an anti-CD40 antibody, or a functional fragment thereof, with silenced ADCC activity that binds both the xenograft organ and the human CD40 and said binding inhibits CD40L induced signaling with no or low agonist activity with respect to CD40 signaling.

The xenograft organ that can be transplanted can be an islet, a heart, a kidney, a cornea, skin, a liver, or a lung. In one embodiment of the disclosure, the xenograft organ is a kidney. The meaning of the term “no or low ADCC activity” is described in the definition section above. The anti-CD40 antibody with silenced ADCC activity can comprise for example a silent Fc IgG1 region. Such a silencing, which abolishes FcγR binding and associated effector functions like ADCC and CDC, can be obtained when using the IgG1 isotype subclass with specific mutations in the Fc region of an antibody known to the person skilled in the art (e.g. Arduin et al., Mol Immunol. 2015 Feb;63(2), DOI: 10.1016/j.molimm.2014.09.017). These Fc silencing mutations for example are leucine (L) to alanine (A) substitution at the position 234 and 235 (LALA) and the alanine (A) to asparagine (N) substitution at position 297 or the D256A mutation. Furthermore, IgG4 version can be used resulting in antibodies that shows neither show antibody-dependent cell-mediated cytotoxicity (ADCC) nor complement-dependent cytotoxicity (CDC).

In relation to the pharmaceutical composition for use in the prevention of graft rejection in a subject receiving a xenograft organ (or the method of suppressing the rejection and prolonging the survival of a xenograft from an animal in a human recipient), the term “CD40 antibody that binds both the xenograft organ CD40 and the human CD40” refers to an antibody that binds to the CD40 polypeptide of the xenograft donor as well as to human CD40 with a KD of about 10 nM, with a KD of about 5 nM or with a KD of about 1 nM.

In another embodiment of the disclosure, the CD40 antibody or a functional fragment thereof that binds both the xenograft organ CD40 and the human CD40 is an antibody or a functional fragment thereof that binds to same human CD40 epitope and to the same xenograft donor organ CD40 epitope as the antibodies described herein. In one embodiment of the disclosure, the CD40 antibody or a functional fragment thereof binds to the same pig xenograft CD40 protein epitope and to the same human CD40 protein epitope, respectively, as CFZ533. In different embodiment of the disclosure, the CD40 antibody or a functional fragment thereof binding the pig xenograft CD40 protein and the human CD40 protein, binds an epitope of both proteins, said epitope being comprised between amino acids 64-120 of SEQ ID NO:37 (human CD40 protein sequence).

Hence, antibodies to be used in the inventive methods and treatments or antibodies that can be used in the inventive therapeutic compositions can therefore be identified based on their ability to cross-compete (e.g., to competitively inhibit the binding of, in a statistically significant manner) with antibodies of the disclosure in standard CD40 binding assays. The ability of a given CD40 antibody or a functional fragment thereof to inhibit the binding of CD40 antibodies disclosed herein, e.g., CFZ533, to human CD40 and pig CD40 demonstrates that said antibody can compete with CFZ533 for binding to human CD40 and pig CD40; such an antibody may fulfil the requirement of binding both the xenograft organ CD40 and the human CD40.

Thus, the disclosure provides a pharmaceutical composition comprising an anti-CD40 antibody or functional fragment thereof for use in the prevention of graft rejection in a subject receiving a xenograft organ, wherein the anti-CD40 antibody is an anti-CD40 antibody, or a functional fragment thereof, (i) with silenced ADCC activity that (ii) binds both the xenograft organ and the human CD40 and (iii) said binding inhibits CD40L induced signaling with (iv) no or low agonist activity with respect to CD40 signaling, wherein said antibody binds to an human CD40 epitope and an pig CD40 epitope recognised by CFZ533.

Following more detailed epitope mapping experiments, the binding regions of antibodies of the disclosure have been more clearly defined. Thus, the disclosure provides pharmaceutical composition comprising an anti-CD40 antibody or functional fragment thereof for use in the prevention of graft rejection in a subject receiving a xenograft organ, wherein the anti-CD40 antibody binds to amino acids in the epitope region comprising amino acids (i) 60-70, (ii) 75-95, and (iii) 115-125 of SEQ ID NO:37 and said binding inhibits CD40L induced signaling with (iv) no or low agonist activity with respect to CD40 signaling.

In one embodiment, the above-mentioned antibody is an anti-CD40 antibody or functional fragment thereof that binds to an epitope consisting the amino acids (i) 64-75, (ii) 79-80, (iii) 82-87, (iv) 94 and (v) 118.-120 of SEQ ID NO:37 and said binding inhibits CD40L induced signaling with (iv) no or low agonist activity with respect to CD40 signaling.

As used herein, the term “binding inhibits CD40L induced signaling” refers to a CD40 antagonist activity which is intended to refer to an antibody or functional fragment thereof that inhibits CD40 induced signaling activity in the presence of CD40L in a human cell assay such as the CD40L-mediated PBMC proliferation assay. In some embodiments, the antibodies or functional fragment thereof inhibit CD40L induced signaling with an 1050 of 50 ng/ml or less, for example with an 1050 of 20ng/m1 or less, as measured in CD40L-mediated PBMC proliferation assay. Methods to analyse the CD40L induced signaling, and blockage thereof, are well known in the art. For example, 1050 values for anti-CD40 antibody mediated inhibition of CD40L can be assessed using a CD40L-mediated proliferation of PBMCs assay as described in detail in the patent application W02012065950 e.g., methods sections 1. CD40L-mediated PBMC proliferation assay; 1.1 Purification of human peripheral blood mononuclear cells (PBMCs) and 1.2 In vitro PBMC stimulation assay, which are herewith incorporated by reference.

In a related specific embodiment of the invention the CD40 antibody comprised in the pharmaceutical composition for use in the prevention of graft rejection in a subject receiving a xenograft organ (or used in the method of suppressing the rejection and prolonging the survival of a xenograft organ from an animal in a human recipient) inhibits the CD40L induced signaling in the human cells/tissue/organ as well as in the xenograft donor cells/tissue/organ with an 1050 of 50 ng/ml or less, for example with an 1050 of 20 ng/ml or less, as measured in CD40L-mediated PBMC proliferation assay.

Methods to prove the CD40L agonistic activity of antibodies and experimental data showing the non-agonistic activity of the antibodies CFZ533 and mAb2 are disclosed in the patent publication Wo2012065950 in the examples in the methods sections 1. CD40L-mediated PBMC proliferation assay; 1.1 Purification of human peripheral blood mononuclear cells (PBMCs) and 1.2 In vitro PBMC stimulation assay, which are herewith incorporated by reference. The experimental results provided in Example 1 of WO2012065950 (“Evaluation of the agonistic activity of mAb1, mAb2 and mAb3”) confirmed that the anti-CD40 antibodies CFZ533 (N297A) and mAb2 (D265A) showed non agonistic CD40L blocking properties. In particular, the experimental results showed that none of the Fc silent anti-CD40 antibodies were capable of stimulating cell division of human PBMCs. Collectively these results demonstrated that neither CFZ533 (N297A) nor mAb2 (D265A) possess agonistic activity.

In a specific embodiment the pharmaceutical composition for use in the prevention of graft rejection in xenotransplantation (or the method of suppressing the rejection and prolonging the survival of a xenograft organ from an animal in a human recipient) comprises the use of an antibody that a) binds to CD40 with a KD of 10 nM or 5 nM or less to human CD40 and CD40 of the xenograft donor; b) inhibits CD40L induced signaling with an 1050 of 100 ng/ml or 50 ng/ml or 20 ng/ml or less as measured in CD40L-mediated PBMC proliferation assay; c) has no or low agonist activity as measured in a bioassay such as CD40L-mediated PBMC proliferation assay and, d) has no or low ADCC activity.

CFZ533 is a human monoclonal antibody directed against human CD40. It belongs to the IgG1 isotype subclass and comprises an Fc-silencing mutation (N297A) which abolishes FcγR binding and associated effector functions like ADCC and CDC. CFZ533 is disclosed in U.S. Pat. Nos. 8,828,396 and 9,221,913. The CDR sequences of CFZ533 are included herein in Table 1: HCDR1 sequence (SEQ ID NO: 25), HCDR2 sequence (SEQ ID NO: 26), HCDR3 sequence (SEQ ID NO: 27), LCDR1 sequence (SEQ ID NO: 28), LCDR2 sequence (SEQ ID NO: 29) and LCDR3 sequence (SEQ ID NO: 30), numbered according to Kabat definition. The VH and VL sequences and full length heavy and light chain sequences are given in Table 1 as SEQ ID Nos: 31-36, respectively.

In another embodiment, the anti-CD40 antibody or a functional fragment thereof to be administered is any antibody having the CDR sequences of CFZ533 (as described in SEQ ID Nos. 25-30) and comprising a Fc region mutation abolishing antibody-dependent cell-mediated cytotoxicity (ADCC) nor complement-dependent cytotoxicity (CDC). The heavy and light chain amino acids of such an antibody (e.g. mAb2) are given in Table 1, SEQ ID Nos: 35 and 36, respectively. mAb2 is another example of a silent IgG1 antibody and comprises a D265A mutation in the Fc region.

In one embodiment of the disclosure (i) the pharmaceutical compositions comprising an anti-CD40 antibody or functional fragment thereof—as described above—for use in the prevention of graft rejection or (ii) the method of suppressing the rejection and prolonging the survival of a xenograft organ from an animal in a human recipient is applied to a subject receiving a pig xenograft organ. In such a situation the anti-CD40 antibody, or a functional fragment thereof, comprised in the used pharmaceutical composition/method has a silenced ADCC activity, inhibits CD40L induced signaling with no or low agonist activity with respect to CD40 signaling and binds the pig CD40 and the human CD40. The xenograft pig donor organisms can be a transgenic organism. In one embodiment of the disclosure, the transgenic donor pig has been genetically modified by disrupting the a(1,3)-galactosyltransferase and cytidine monophosphate-N-acetylneuraminic acid hydroxylase (CMAH) genes. Further approaches to reduce the generation of donor specific antibodies in xenotransplant recipients have focused on the reduction or deletion of xenoantigens present on the organ endothelium but which are not found in humans. The Galα(1,3)-Gal antigen, encoded by the GGTA1 gene, is found on the surface of porcine endothelial cells but is not produced in humans or old world monkeys. αGal has been recognized as the most important xenoantigen in the pig, representing 70-85% of all human xenoreactive antibodies against pig cells. The recognition and binding of the Galα(1,3)Gal antigen by xenoreactive antibodies activates the classical complement pathway, leading to the formation of the membrane attack complex (MAC), which acts as a catalyst for cell membrane penetration by proteins forming transmembrane channels, ultimately resulting in cell lysis (Platt et al (1991) Transplantation 52: 214-20).

Creation of GGTA1^{-/-} pigs and organs therefrom was hypothesised to solve the hyperacute rejection observed in xenotransplantation. Transplantation of GGTA1^{-/-} pig kidneys into non-human primates using rATG (Thymoglobulin), tacrolimus, and mycophenolic acid as immunosuppression was performed; however, the kidneys in this series were rejected in 8-16 days, and xenoreactive antibodies still initiated complement activation leading to interstitial hemorrhage and thrombotic microangiopathy in rejected kidneys (Chen G et al., (2005) Nature Med. 11(12): 1295). This work showed that non-Gal xenoantigens remained as a barrier to moving forward with clinical xenotransplantation.

Subsequent work therefore focused on the elimination of additional surface xenoreactive antigens from porcine cells include cytidine monophosphate-N-acetylneuraminic acid hydroxylase (CMAH), which catalyses the reaction producing the Neu5Gc antigen (N-glycolylneuraminic acid), and the glycan produced by the β1,4-N-acetylgalactosaminyl transferase (β4GalNT2) enzyme activity. Use of recent advances in gene editing technologies such as zinc finger nucleases (ZFN), transcription activator-like effector nucleases (TALEN) and modifications using the CRISPR/Cas9 system have enabled the inactivation of the genes encoding the afore mentioned xenoreactive antigens and have led to, for example, the generation of double and triple knock out pigs lacking the GGTA1, CMAH and/or p4GalNT2 genes (U.S. Pat. Nos. 7,795, 493; 9,888,674; WO2016065046).

In one embodiment, the following transgenic pig are preferable xenotransplant organ donors used in combination with (i) the herein disclosed pharmaceutical composition for use in the prevention of xenograft rejection or (ii) the method of suppressing the rejection/prolonging the survival of a xenograft organ from a pig in a human recipient:

1) If the xenotransplant organ is a heart, GTKO/β4GalNT2-KO pigs (M. M. Mohiuddin, A. K. Singh, P. C. Corcoran, M. L. Thomas Iii, T. Clark, B. G. Lewis, R. F. Hoyt, M. Eckhaus, R. N. Pierson Iii, A. J. Belli, E. Wolf, N. Klymiuk, C. Phelps, K. A. Reimann, D. Ayares, K. A. Horvath, Chimeric 2C10R4 anti-CD40 antibody therapy is critical for long-term survival of GTKO.hCD46.hTBM pig-to-primate cardiac xenograft, Nat. Commun. 7 (2016) 11138.).

2) If the xenotransplant organ is a kidney, a) GTKO/hCD55 pigs (M. Wijkstrom, H. Iwase, W. Paris, H. Hara, M. Ezzelarab, D. K. Cooper, Renal xenotransplantation: experimental progress and clinical prospects, Kidney Int. 91 (2017) 790-796),

or

b) GTKO/hCD46/hCD55/hEPCR/hTFPI/hCD47 pigs (H. Iwase, H. Hara, M. Ezzelarab, T. Li, Z. Zhang, B. Gao, H. Liu, C. Long, Y. Wang, A. Cassano, E. Klein, C. Phelps, D. Ayares, A. Humar, M. Wijkstrom, D. K. C. Cooper; Immunological and physiological observations in baboons with life-supporting genetically engineered pig kidney grafts, Xenotransplantation 24 (2017), e12293),

or

c) GTKO/β4GalNT2-KO pigs (A. B. Adams, S. C. Kim, G. R. Martens, J. M. Ladowski, J. L. Estrada, L. M. Reyes, C. Breeden, A. Stephenson, D. E. Eckhoff, M. Tector, A. J. Tector, Xenoantigen deletion and chemical immunosuppression can prolong renal xenograft survival, Ann. Surg. 268 (2018) 564-573).

3) If the xenotransplant organ is a skin, GTKO/hCD47 pigs (A. A. Tena, D. H. Sachs, C. Mallard, Y. G. Yang, M. Tasaki, E. Farkash, I. A. Rosales, R. B. Colvin, D. A. Leonard, R. J. Hawley, Prolonged Survival of Pig Skin on Baboons After Administration of Pig Cells Expressing Human CD47, Transplantation 101 (2017) 316-321).

4) if the xenotransplant organ is a liver, GTKO pigs (J. A. Shah, N. Navarro-Alvarez, M. DeFazio, I. A. Rosales, N. Elias, H. Yeh, R. B. Colvin, A. B. Cosimi, J. F. Markmann, M. Hertl, D. H. Sachs, P. A. Vagefi, A bridge to somewhere: 25-day survival after pig-to-baboon liver xenotransplantation, Ann. Surg.263 (2016) 1069-1071).

5) if the xenotransplant organ is a lung, GTKO/β4GalNT2-KO/hCD46/hCD47/hEPCR/hTBM/hHO-1 pigs

(L. Burdorf, C. Laird, S. Sendil, N. O∝Neill, D. Parsell, I. Tatarov, T. S. Zhang, A. Cimeno, C. J. Phelps, D. L. Ayares, A. M. Azimzadeh, R. N. Pierson, Progress in xenogeneic lung transplantation using multi-transgenic donor pigs and targeted supportive drug treatments, Transplantation 102 (2018) S106.; L. Burdorf, C. Laird, S. Sendil, N. O'Neill, T. Zhang, D. Parsell, I. Tatarov, Z. Abady, B. M. Cerel, S. Pratts, C. J. Phelps, D. L. Ayares, A. M. Azimzadeh, R. N. Pierson, 31 Day xeno lung recipient survival—progress towards the clinic, J. Heart Lung Transplant. 38 (2019) S39.).

In one embodiment, wherein the xenotransplant organ is a kidney, a transgenic pic comprising at least the GTKO/hCD55 pic mutations will be used as an organ donor and the subject receiving said organ is treated with a pharmaceutical composition comprising an anti-CD40 antibody, or a functional fragment thereof, to prevent the xenograft rejection as disclosed herein, wherein said anti-CD40 antibody has a silenced ADCC activity, inhibits CD40L induced signaling with no or low agonist activity with respect to CD40 signaling and binds the pig CD40 and the human CD40. In one embodiment said anti-CD40 antibody is iscalimab.

In another embodiment, wherein the xenotransplant organ is a kidney, a transgenic pic comprising at least the GTKO/hCD55 pic mutations will be used as an organ donor and the subject receiving said organ is treated with a pharmaceutical composition comprising a silenced anti-CD40 antibody and an anti-C5 antibody, or a functional fragment thereof to prevent the xenograft rejection to prevent the xenograft rejection as disclosed herein, wherein said anti-CD40 antibody has a silenced ADCC activity, inhibits CD40L induced signaling with no or low agonist activity with respect to CD40 signaling and binds the pig CD40 and the human CD40. In one embodiment said anti-CD40 antibody is iscalimab and the anti-CD5 antibody is tesidolumab or eculizimab.

In an embodiment of the present disclosure, the pharmaceutical composition comprising an anti-CD40 antibody or functional fragment thereof—as described above—for use in the prevention of xenograft rejection (or for use in the method of suppressing the rejection/prolonging the survival of a xenograft organ) in a subject (e.g. a pig organ, like a pig kidney), is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 30 mg anti-CD40 antibody or a functional fragment thereof per kg of the subject, followed by a maintenance dose which is between 300 mg and 600 mg. The antibody comprised in said composition, e.g. iscalimab, or an antigen binding/functional fragment thereof, is administered to the subject at a loading dose, e.g. before, at the time of or after the xenotransplant, e.g. up to 12 hours, up to 10 hours, up to 8 hours, up to 6 hours up to 4 hours, up to 2 hours or up to one hour prior to xenotransplantation, at the time of xenotransplantation or up to 1 hour, up to 2 hours, up to 4 hours, up to 6 hours, up to 8 hours, up to 10 hours or up to 12 hours post xenotransplantation.

The loading dose of anti-CD40 antibody or an antigen binding/functional fragment thereof may be between about 5-100 mg/kg, between about 10-50 mg/kg, may be about 10 mg/kg, about 20 mg/kg, about 30 mg/kg or about 40 mg/kg. In certain embodiments, the loading dose is 30 mg/kg. In some embodiments, the loading dose is administered once or 2, 3, 4, 5, 6 or more times, 1 to 3, 1 to 4, 2 to 4, 2 to 5, 2 to 6, 3 to 6, 4 to 6 or 6 to 8 times. In some embodiments, the loading dose is administered 1, 2, 3, 4, 5, 6 or more times on one day, over 1 to 3 days, 3 to 5 days, 5 to 7 days, 5 to 10 days, 7 to 12 days, 7 to 14 days, 7 to 21 days or 14 to 21 days. In certain embodiments, the loading dose is administered once on the day of xenotransplantation.

The loading dose of the anti-CD40 antibody or an antigen binding/functional fragment thereof may be administered prior to the administration of a maintenance dose. In some embodiments, the loading dose is 1.2, 1.25, 1.3, 1.35, 1.4, 1.45, 1.5, 2, 2.5, 3, 3.5, 4, 4.5, 5, 5.5, or 6 times higher than the maintenance dose, or 1.2 to 2, 2 to 3, 2 to 4, 2 to 6, 3 to 4, 3 to 6, or 4 to 6 times higher than the maintenance dose. In one embodiment, the loading dose is three times higher than the maintenance dose.

In an embodiment of the present disclosure, the pharmaceutical composition comprising an anti-CD40 antibody or functional fragment thereof—as described above—for use in the prevention of xenograft rejection (or for use in the method of suppressing the rejection/prolonging the survival of a xenograft organ) is administered to the subject as a maintenance dose. The maintenance dose is comprised of between 1 mg/kg and 50 mg/kg, between 5 mg/kg and 30 mg/kg, between 8 mg/kg and 20 mg/kg, or is about 10 mg/kg.

In certain embodiments, the maintenance dose is administered once or 2, 3, 4, 5, 6 or more times, or from 1 to 3, 1 to 4, 2 to 4, 2 to 5, 2 to 6, 3 to 6, 4 to 6, 6 to 8, or more times.

In some embodiments, the maintenance dose is administered at least twice a week, weekly, at least every two weeks, at least monthly.

The period during which the maintenance dose is administered to the subject is herein referred to as the maintenance period. During the maintenance period, the maintenance dose can be supplemented by at least one supplemental dose, as described herein below. The maintenance period can start prior to transplantation, on the day of transplantation or after the transplantation, e.g. one week, two weeks or one month after the xenotransplantation. The duration of administration of the maintenance dose, e.g. duration of the maintenance period, is at least 2 weeks, at least 4 weeks, at least 6 weeks, at least 8 weeks, at least 10 weeks, at least 3 months, at least 6 months, at least 9 months, at least one year or can be lifelong. The maintenance period can last until the xenotransplant recipient needs a new transplant.

In some embodiments, the antibody comprised in the pharmaceutical composition for use in the prevention of xenograft rejection (or for use in the method of suppressing the rejection/prolonging the survival of a xenograft organ), e.g., iscalimab, or an antigen binding/functional fragment thereof, is administered in such a way that a constant serum trough level of said antibody or an antigen binding/functional fragment thereof is achieved. As herein defined, serum trough level of the anti-CD40 antibody or antigen binding/functional fragment thereof refers to the serum trough level of total antibody (or an antigen binding/functional fragment thereof), free antibody or bound antibody, e.g. to total antibody (i.e. antibody that is free plus antibody that is bound to the CD40 protein).

In some embodiments, the antibody comprised in the pharmaceutical composition for use in the prevention of xenograft rejection, e.g. iscalimab, or an antigen binding/functional fragment thereof (or for use in the method of suppressing the rejection/prolonging the survival of a xenograft organ), is administered in such a way that a constant serum trough level of said antibody or antigen binding/functional fragment thereof of 30-100 μg/mL is maintained, such as 40-100 μg/mL, 50-100 μg/mL, 55-100 μg/mL or about 50-60 μg/mL.

In other embodiments of the disclosure, the antibody comprised in the pharmaceutical composition for use in the prevention of xenograft rejection, e.g. iscalimab, or an antigen binding/functional fragment thereof (or for use in the method of suppressing the rejection/prolonging the survival of a xenograft organ), is administered in such a way that a constant serum trough concentration of at least 30 μg/mL, at least 40 μg/mL, at least 50 μg/mL, at least 55 μg/mL, at least100 μg/mL, or at least 200 μg/mL, is achieved.

In some embodiments, the dose of the antibody comprised in the pharmaceutical composition for use in the prevention of xenograft rejection, e.g. iscalimab, or an antigen binding/functional fragment thereof (or for use in the method of suppressing the rejection/prolonging the survival of a xenograft organ), may be increased if the trough concentration (e.g. in serum) of the anti-CD40 antibody or an antigen binding/functional fragment thereof (e.g. of total antibody) in the subject is below 10 μg/mL, below 20 μg/mL, below 30 μg/mL, below 40 μg/mL, below 50 μg/mL, below 60 μg/mL, below 70 μg/mL, below 80 μg/mL, below 90 μg/mL or below 100 μg/mL.

In some embodiments, the dose of the antibody comprised in the pharmaceutical composition for use in the prevention of graft rejection, e.g. iscalimab, or an antigen binding/functional fragment thereof (or for use in the method of suppressing the rejection/prolonging the survival of a xenograft organ) is decreased if the trough concentration (e.g. in serum) of the anti-CD40 antibody or an antigen binding/functional fragment thereof (e.g. of total antibody) from the subject is above 50 μg/mL, above 55 μg/mL, above 100 μg/mL, above 150 μg/mL, above 200 μg/mL, above 300 μg/mL, above 400 μg/mL or above 500 μg/mL.

In some embodiments, the dose of the antibody comprised in the pharmaceutical composition for use in the prevention of xenograft rejection (or for use in the method of suppressing the rejection/prolonging the survival of a xenograft organ), e.g. iscalimab, or an antigen binding/functional fragment thereof is maintained if the trough concentration (e.g. in serum) of the anti-CD40 antibody or an antigen binding/functional fragment thereof (e.g. of total antibody) from the subject is 10-100 μg/mL, 50-100 μg/mL or 55-100 μg/mL.

According to the present disclosure, the CD40 antibody comprised in the pharmaceutical composition for use in the prevention of xenograft rejection (or for use in the method of suppressing the rejection/prolonging the survival of a xenograft organ), e.g., iscalimab, or an antigen binding/functional fragment thereof, is administered to a subject at the maintenance dose at least weekly, or at least every two weeks or at least monthly. The maintenance dose can be administered over a period of at least 6 weeks, at least 8 weeks, at least 10 weeks, at least 3 months, at least 6 months, at least 9 months, at least one year or lifelong.

In one embodiment, the CD40 antibody comprised in the pharmaceutical composition for use in the prevention of xenograft rejection (or for use in the method of suppressing the rejection/prolonging the survival of a xenograft organ), e.g., iscalimab, or an antigen binding/functional fragment thereof is, is administered to a subject during a weekly maintenance period at a dose of about 10 mg/kg. The period during which the maintenance dose is administered lasts for a period of at least 10 weeks.

According to the disclosure, the CD40 antibody comprised in the pharmaceutical composition for use in the prevention of xenograft rejection (or for use in the method of suppressing the rejection/prolonging the survival of a xenograft organ), e.g. iscalimab, or an antigen binding/functional fragment thereof is provided at a dose such that the concentrations of serum CD40 antibody, e.g. constant serum trough level at steady-state of antibody, e.g. constant serum trough level at steady-state of total antibody, is comprised between 30 and 100 μg/mL, 50 and 100 μg/mL, 55 to 100 μg/mL, 40 to 60 μg/mL or 45 to 55 μg/mL. For example, the concentration of total serum antibody, e.g., constant serum trough level at steady-state of total antibody, is about 100 pg/mL, about 60 μg/mL or about 50 μg/mL.

According to the present disclosure, the CD40 antibody comprised in the pharmaceutical composition for use in the prevention of xenograft rejection (or for use in the method of suppressing the rejection/prolonging the survival of a xenograft organ), e.g., iscalimab or an antigen binding/functional fragment thereof, is administered repeatedly.

According to the disclosure, the interval between two consecutive administrations (e.g., of maintenance dose) may vary during the treatment, e.g., may be of 1 week or two weeks, and then may increase, e.g. may double, may then be of 2 weeks or one month.

To maximize efficacy and minimize adverse effects, current immunosuppressant (IS) regimens use combinations of IS drugs. Care is taken to achieve synergy or additive immunosuppressive effects via the administration of submaximal doses of individual agents with different mechanism of actions while avoiding overlapping toxicities. Most treatment regimens today include two or more primary and adjunct IS with or without an induction agent. Induction agents are administered during the first hours to days post transplantation to suppress the recipient's immune system and priming of an immune response to the allograft while the other IS agents are reaching effective concentrations. Induction agents include the anti-CD25 mAb basiliximab (Simulect®, Novartis) or polyclonal anti-T cell globulin (Thymoglobulin®, rabbit ATG, rATG, Genzyme). In highly sensitized patients, induction with an anti-CD52 mAb, alemtuzumab (Campath®, Sanofi-Aventis SA) which leads to long-term lymphocyte depletion has been used. Within 1-2 days following transplant, the maintenance treatment regimen is initiated with two or more of the following agents: a calcineurin inhibitor (CNI) such as cyclosporine (CsA, Neoral®, Novartis) or tacrolimus (Tac, FK506, Prograf®, Astellas), together with a lymphocyte proliferation inhibitor such as mycophenolic acid (MPA; Myfortic®, Novartis) or mycophenolate mofetil (MMF; CellCept®, Roche) or proliferation signal inhibitor such as everolimus (Zortress®, Certican®, Novartis) or sirolimus (Rapamune®, Pfizer). More recently, the T cell co-stimulation blocker belatacept Nulojix®, BMS), a fusion protein, demonstrated the potential of a biologic agent to replace CNIs in a calcineurin-free treatment regimen with MPA. In one embodiment of the disclosure the pharmaceutical composition comprising an anti-CD40 antibody or functional fragment thereof—as described above, e.g. iscalimab, or an antigen binding/functional fragment thereof—for use in the prevention of xenograft rejection (or for use in the method of suppressing the rejection/prolonging the survival of a xenograft organ) in a subject, is administered to a subject that has received an induction therapy prior to receiving the xenotransplant, such an induction therapy could comprise the administration of e.g. an anti-CD4 antibody and/or an anti-CD20 antibody.

In one embodiment of the disclosure the pharmaceutical composition comprising an anti-CD40 antibody or functional fragment thereof—as described above, e.g. iscalimab, or an antigen binding/functional fragment thereof—for use in the prevention of xenograft rejection (or for use in the method of suppressing the rejection/prolonging the survival of a xenograft organ) in a subject, is combined with other anti-proliferative agents like mycophenolate mofetil (MMF), or steroids, like prednisone or T cell immunosuppressive compounds, like calcineurin inhibitors such as cyclosporin and tacrolimus.

In one embodiment of the disclosure (i) the pharmaceutical composition for use in the prevention of xenograft organ loss in solid organ transplantation patients comprising a CD40 antibody -as described above e.g. CFZ533, mAb2- or (ii) the method of suppressing the rejection/prolonging the survival of a xenograft organ comprising administering to the human recipient an anti-CD40 antibody or functional fragment thereof—as described above—is used in combination with two or more of the following agents: a calcineurin inhibitor (CNI) such as cyclosporine (CsA, Neoral®, Novartis) or tacrolimus (Tac, FK506, Prograf®, Astellas), a lymphocyte proliferation inhibitor such as mycophenolic acid (MPA; Myfortic®, Novartis) or mycophenolate mofetil (MMF; CellCept®, Roche) or proliferation signal inhibitor such as everolimus (Zortress®, Certican®, Novartis) or sirolimus (Rapamune®, Pfizer) or a T cell co-stimulation blocker such as belatacept (Nulojix®, BMS). In one embodiment of the disclosure (i) the pharmaceutical composition for use in the prevention of xenograft organ loss in solid organ transplantation patients comprising a CD40 antibody—as described above (e.g. CFZ533 or mAb2) or (ii) the method of suppressing the rejection/prolonging the survival of a xenograft organ comprising administering to the human recipient an anti-CD40 antibody or functional fragment thereof—as described above (e.g. CFZ533 or mAb2), is used in combination with a T cell co-stimulation blocker such as belatacept (Nulojix®, BMS) in a calcineurin-free treatment regimen. In one embodiment of the disclosure (i) the pharmaceutical composition for use in the prevention of xenograft organ loss in solid organ transplantation patients comprising a CD40 antibody—as described above (e.g. CFZ533 or mAb2) or (ii) the method of suppressing the rejection/prolonging the survival of a xenograft organ comprising administering to the human recipient an anti-CD40 antibody or functional fragment thereof—as described above (e.g. CFZ533 or mAb2), is used in combination with CsA, (Neoral®, Novartis), tacrolimus (Tac, FK506, Prograf®, Astellas) and/or a mTor inhibitor such as everolimus (Zortress®, Certican®, Novartis).

The pharmaceutical composition comprising a CD40 antibody—as described above—may be suitable for use in the prevention of graft rejection in solid organ transplantation, and particularly prevention of graft rejection in kidney transplantation, liver transplantation, heart transplantation, lung transplantation, pancreas transplantation, intestine transplantation or composite tissue transplantation.

2. Pharmaceutical Compositions Comprising an Anti-C5 Antibody, or the Functional Fragment Thereof and an Anti-CD40 Antibody, or the Functional Fragment Thereof

In previous studies, C5 blockade through the administration of the anti-C5 antibody eculizumab (Soliris®) has been investigated as a strategy for the prevention or treatment of AMR in kidney allotransplantation (Johnson & Leca (2015) Curr Opin Organ Transplant. 20(6): 643-51; Stegall et al, (2011) American Journal of Transplantation 11: 2405-2413; Cornell et al, (2015) American Journal of Transplantation 15: 1293-1302). In the setting of persistently high DSA concentrations, such as those who received long-term eculizumab treatment, eculizumab failed to prevent the development of subclinical inflammation and chronic, microcirculatory injury, although outcomes were favourable if post-transplant antibody levels were low (Johnson et al., (2015) Curr Opin Organ Transplant. 20(6): 643-51).

In the present disclosure, it was found that an anti-C5 antibody or an antigen binding/functional fragment thereof, such as e.g., tesidolumab or eculizumab are suitable for the treatment or prevention of AMR or an associated condition, in particular in the treatment or prevention of AMR in a subject receiving a xenograft, in combination with an anti-CD40 antibody.

Therefore, in another aspect the disclosure relates to a pharmaceutical composition comprising an anti-CD40 antibody and an anti-C5 antibody (or functional fragments thereof) in combination with at least a pharmaceutically acceptable excipient, carrier or diluent.

In one embodiment, the anti-CD40 antibody comprised in the above-described pharmaceutical composition is an anti-CD40 antibody, or a functional fragment thereof, (i) with silenced ADCC activity that (ii) binds both the xenograft organ and the human CD40 and (iii) said binding inhibits CD40L induced signaling with (iv) no or low agonist activity with respect to CD40 signaling.

In another embodiment of the disclosure the above described CD40 antibody comprised in the inventive pharmaceutical composition comprises the following CDR sequences: HCDR1 sequence (SEQ ID NO: 25), HCDR2 sequence (SEQ ID NO: 26), HCDR3 sequence (SEQ ID NO: 27), LCDR1 sequence (SEQ ID NO: 28), LCDR2 sequence (SEQ ID NO: 29) and LCDR3 sequence (SEQ ID NO: 30), numbered according to Kabat definition. In another preferred embodiments said CD40 antibody comprises the VH and VL sequences and full length heavy and light chain sequences are given in Table 1 as SEQ ID Nos: 31-36, respectively. In yet another embodiment, said anti-CD40 comprises the heavy and light chain amino acids according to SEQ ID Nos: 35 and 36, respectively.

In one embodiment of the present disclosure, the anti-C5 antibody comprised in the disclosed pharmaceutical composition in combination with an anti-CD40 antibody or functional fragment thereof binds to the alpha chain of the C5 complement protein; it may inhibit cleavage of C5 complement protein, e.g., inhibits the generation of C5b and C5a. The anti-C5 antibody may bind to the C5a epitope on intact or cleaved C5/C5a; it may neutralize the activity of C5a without preventing cleavage of C5. In another embodiment, the anti-C5 antibody to be administered binds to CSaR, e.g., inhibiting binding of C5a to CSaR.

Tesidolumab is a recombinant, high-affinity, human monoclonal antibody of the IgG1/lambda isotype, which binds to C5 and neutralizes its activity in the complement cascade. As described previously, C5 serves as a central node necessary for the generation of C5a as well as the formation of the membrane attack complex (MAC).

Tesidolumab is described in Intl. Pat. Appl. No. WO 2010/015608, “Compositions and Methods for Antibodies Targeting Complement Protein C5” and U.S. Pat. No. 8,241,628. The CDR sequences of tesidolumab are included herein in Table 1: HCDR1 sequence (SEQ ID NO: 1), HCDR2 sequence (SEQ ID NO: 2), HCDR3 sequence (SEQ ID NO: 3), LCDR1 sequence (SEQ ID NO: 4), LCDR2 sequence (SEQ ID NO: 5) and LCDR3 sequence (SEQ ID NO: 6), numbered according to Kabat definition. The VH and VL sequences and full length heavy and light chain sequences are given in Table 1 as SEQ ID Nos: 7-10, respectively.

Therefore, in one embodiment, the disclosure relates to a pharmaceutical composition comprising an anti-CD40 antibody and tesidolumab in combination with at least a pharmaceutically acceptable excipient, carrier or diluent. In yet another embodiment, the disclosure relates to a pharmaceutical composition comprising CFZ533 and tesidolumab in combination with at least a pharmaceutically acceptable excipient, carrier or diluent.

In another embodiment, the anti-C5 antibody combined with the anti-CD40 antibody in the disclosed pharmaceutical composition is an antibody having the CDR sequences of tesidolumab, as described in SEQ ID Nos. 1-6. Further examples of anti-C5 antibodies combined with the anti-CD40 antibody in the disclosed pharmaceutical composition include the humanized monoclonal antibody eculizumab (Soliris®), the antibody fragment pexelizumab and ALXN1210 (ravulizumab). Pexelizumab (Alexion Pharmaceuticals), also called 5G1.1, is a recombinant, single-chain, anti-C5 monoclonal antibody (Shernan et al., (2004) Ann Thorac Surg. 77(3): 942-9, discussion 949-50). ALXN1210 (Alexion Pharmaceuticals) is an extended half-life version of eculizumab with targeted substitutions to reduce target-mediated drug disposition and enhance FcRn-mediated recycling (Sheridan et al. (2018) PLoS ONE 13(4): e0195909).

Therefore, in one embodiment, the disclosure relates to a pharmaceutical composition comprising an anti-CD40 antibody or functional fragment thereof and eculizumab in combination with at least a pharmaceutically acceptable excipient, carrier or diluent. In yet another embodiment, the disclosure relates to a pharmaceutical composition comprising CFZ533 and eculizumab in combination with at least a pharmaceutically acceptable excipient, carrier or diluent.

The CDR sequences, VH, VL and heavy and light chain sequences of eculizumab are shown in SEQ ID NOs: 11 to 20. Other anti-C5 antibodies that could be comprised in the inventive pharmaceutical composition in combination with an anti-CD40 antibody or functional fragment thereof are variant antibodies of eculizumab such as those described in WO2015/134894 from Alexion Pharmaceuticals, Inc. In particular, the eculizumab variant antibody is BNJ441 having the heavy and light chain sequences as shown in SEQ ID NOs: 21 and 22, respectively or eculizumab variant antibody ALXN1210 having the heavy and light chain sequences as shown in SEQ ID Nos: 23 and 24, respectively. Additional anti-C5 antibodies that could be comprised in the inventive pharmaceutical composition in combination with an anti-CD40 antibody or functional fragment thereof are described in Intl. Pat. Appl. No. WO1995/29697 (Alexion Pharmaceuticals), WO2011/37362 (Alexion Pharmaceuticals), WO2011/37395 (Alexion Pharmaceuticals) or WO2014/110438 (Alexion Pharmaceuticals).

In another embodiment, the anti-C5 antibody that could be comprised in the inventive pharmaceutical composition in combination with an anti-CD40 antibody or functional fragment thereof binds to a different site on the C5 complement protein than eculizumab, e.g., is anti-C5 monoclonal antibody N19-8 is an (Wurzner et al. (1991) Complement Inflamm. 8:328-40). In yet another embodiment, the anti-C5 antibody to be comprised in the inventive pharmaceutical composition in combination with an anti-CD40 antibody or functional fragment thereof is an anti-C5 aptamer, e.g., ARC1905 (Archemix, Zimura® from Ophthotech) or antibodies related thereto (e.g. ARC186 and ARC187), e.g. as described in WO2007/103549. In yet another embodiment, the anti-C5 antibody to be comprised in the inventive pharmaceutical composition in combination with an anti-CD40 antibody or functional fragment thereof is MubodinaTM/Ergidina from Adienne. Ergidina is a recombinant human minibody (a scFv engineered) against complement component C5 fused with RGD-motif (ADIENNE Pharma & Biotech Press Release 2009, February 4; ADIENNE Pharma & Biotech Press Release 2009, January 20; Noris M et al (2012) Nature Revs Nephrology, 8: 622-33).

The pharmaceutical composition comprising an anti-CD40 antibody such as mAb1 or mAb2 and an anti-C5 antibody such as tesidolumab or eculizumab can comprise pharmaceutically acceptable carrier, various diluents, fillers, salts, buffers, stabilizers, solubilizers, and other materials well known in the art. The characteristics of the carrier will depend on the route of administration. Therapeutic antibodies are typically formulated either in aqueous form ready for administration or as lyophilisate for reconstitution with a suitable diluent prior to administration. A pharmaceutical composition comprising anti-CD40 antibody and an anti-C5 antibody, as described above, may be formulated either as a lyophilisate, or as an aqueous composition, for example in pre-filled syringes. Suitable formulation can provide an aqueous pharmaceutical composition or a lyophilisate that can be reconstituted to give a solution with a high concentration of the antibody active ingredient and a low level of antibody aggregation for delivery to a patient. High concentrations of antibodies are useful as they reduce the amount of material that must be delivered to a patient. Reduced dosing volumes minimize the time taken to deliver a fixed dose to the patient. The aqueous compositions of the invention with high concentration of antibodies are particularly suitable for subcutaneous administration. The pharmaceutical compositions—e.g., for use in the disclosed methods or treatment—may also contain additional therapeutic agents for treatment of the targeted disorder. The above-described compositions can also be formulated to comprise only anti-CD40 antibody such as mAb1 or mAb2 (or functional fragments thereof) or only an anti-C5 antibody such as tesidolumab or eculizumab (or functional fragments thereof). Such formulations are particularly preferred in the combination treatments herein disclosed in section 4 below. Ways to formulate the anti CD40 antibodies CFZ533 and mAb2 are known in the art and have been disclosed in the WO publication WO2013/164789, which specific formulation examples are herein incorporated by reference. In one embodiment, the concentration of the anti-CD40 antibody and the anti-C5 antibody in the aqueous pharmaceutical composition of the invention is at least 50 mg/ml per antibody. In one embodiment, the concentration is at least 100 mg/ml per antibody. In one embodiment, the concentration is at least 150 mg/ml per antibody (wherein the compositions can also be formulated to comprise only an anti-CD40 antibody or only an anti-C5 antibody, or functional fragments thereof). In one embodiment, the concentration is at least 200 mg/ml per antibody. In one embodiment, the concentration is at least 250 mg/ml per antibody (wherein the compositions can also be formulated to comprise only an anti-CD40 antibody or only an anti-C5 antibody, or functional fragments thereof). In one embodiment, the concentration is at least 300 mg/mi per antibody (wherein the compositions can also be formulated to comprise only an anti-CD40 antibody or only an anti-C5 antibody, or functional fragments thereof).

In one embodiment of the disclosure, the aqueous pharmaceutical composition of the invention comprises between 50 mg/ml and 300 mg/ml of an anti-CD40 antibody, for example, iscalimab or mAb2and between 50 mg/ml and 300 mg/ml of an anti-C5 antibody, for example tesidolumab (wherein said compositions can also be formulated to comprise only the anti-CD40 antibody or only the anti-C5 antibody. Such formulations are particularly preferred in the combination treatments herein disclosed in section 4 below).

In one embodiment of the disclosure, the aqueous pharmaceutical composition of the invention comprises between 75 mg/ml and 250 mg/ml of an anti-CD40 antibody, for example, iscalimab or mAb2 and between 50 mg/ml and 300 mg/ml of an anti-C5 antibody, for example tesidolumab (wherein said compositions can also be formulated to comprise only the anti-CD40 antibody or only the anti-C5 antibody. Such formulations are particularly preferred in the combination treatments herein disclosed in section 4 below).

In one embodiment, the aqueous pharmaceutical composition of the invention comprises between 100 mg/ml and 250 mg/ml of an anti-CD40 antibody, for example, iscalimab or mAb2 and between 100 mg/ml and 250 mg/ml of an anti-C5 antibody, for example tesidolumab (wherein said compositions can also be formulated to comprise only the anti-CD40 antibody or only the anti-C5 antibody. Such formulations are particularly preferred in the combination treatments herein disclosed in section 4 below).

In one embodiment, the aqueous pharmaceutical composition of the invention comprises between 100 mg/ml and 200 mg/ml of an anti-CD40 antibody, for example, iscalimab or mAb2 and between 100 mg/ml and 200 mg/ml of an anti-C5 antibody, for example tesidolumab (wherein said compositions may also be formulated to comprise only the anti-CD40 antibody or only the anti-C5 antibody. Such formulations are particularly preferred in the combination treatments herein disclosed in section 4 below).

In one embodiment, the aqueous pharmaceutical composition of the invention comprises about 150 mg/ml of an anti-CD40 antibody, for example, iscalimab or mAb2 and/or about 150 mg/ml of an anti-C5 antibody, for example tesidolumab (wherein said compositions may also be formulated to comprise only the anti-CD40 antibody or only the anti-C5 antibody. Such formulations are particularly preferred in the combination treatments herein disclosed in section 4 below).

In one embodiment, the aqueous pharmaceutical composition of the invention comprises about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml or about 300 mg/mi of an anti-CD40 antibody, for example, iscalimab or mAb2 and comprises about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml or about 300 mg/ml of an anti-C5 antibody, for example tesidolumab (wherein said compositions may also be formulated to comprise only the anti-CD40 antibody or only the anti-C5 antibody. Such formulations are particularly preferred in the combination treatments herein disclosed in section 4 below).

The aqueous pharmaceutical compositions may include, in addition to the anti-CD40 antibody and/or anti-C5 antibody, further components such as one or more of the following: (i) a stabiliser; (ii) a buffering agent; (iii) a surfactant; and (iv) a free amino acid (wherein said compositions may be formulated to comprise only the anti-CD40 antibody or only the anti-C5 antibody or functional fragments thereof. Such formulations are particularly preferred in the combination treatments herein disclosed in section 4 below).

Suitable stabilisers for use in the disclosed pharmaceutical compositions can act, e.g., as viscosity enhancing agents, bulking agents, solubilising agents, and/or the like. The stabiliser can be ionic or non-ionic (e.g. sugars). As sugars they include, but are not limited to, monosaccharides, e.g., fructose, maltose, galactose, glucose, D-mannose, sorbose and the like; disaccharides, e.g., lactose, sucrose, trehalose, cellobiose, and the like; polysaccharides, e.g. raffinose, melezitose, maltodextrins, dextrans, starches, and the like; and alditols, such as mannitol, xylitol, maltitol, lactitol, xylitol sorbitol (glucitol) and the like. For example, the sugar may be sucrose, trehalose, raffinose, maltose, sorbitol or mannitol. The sugar may be a sugar alcohol or an amino sugar. Sucrose is particularly useful. As ionic stabiliser they include salts such as NaCl or amino acid components such as arginine-HCl.

Suitable buffering agents for use with the invention include, but are not limited to, organic acid salts such as salts of citric acid, ascorbic acid, gluconic acid, carbonic acid, tartaric acid, succinic acid, acetic acid or phtalic acid; Tris, thomethamine hydrochloride, or phosphate buffer. In addition, amino acid components can also be used as buffering agent. Such amino acid component includes without limitation glycine and histidine. A histidine buffer is particularly useful.

The aqueous pharmaceutical compositions of the invention or pharmaceutical compositions for use in the inventive combination of anti-CD40 antibody and an anti-C5 antibody include such buffering agent or pH adjusting agent to provide improved pH control. In one embodiment, an aqueous pharmaceutical composition of the invention (or a pharmaceutical composition for use in the inventive combination of anti-CD40 antibody and an anti-C5 antibody) has a pH between 5.0 and 8.0, between 5.5 and 7.5, between 5.0 and 7.0, between 6.0 and 8.0, or between 6.0 and 7.0. In a specific embodiment, an aqueous pharmaceutical composition of the invention has a pH of about 6.0.

As used herein, the term “surfactant” herein refers to organic substances having amphipathic structures; i.e., they are composed of groups of opposing solubility tendencies, typically an oil-soluble hydrocarbon chain and a water-soluble ionic group. Surfactants can be classified, depending on the charge of the surface-active moiety, into anionic, cationic and dispersing agents for various pharmaceutical compositions and preparations of biological materials.

Suitable surfactants for use with the invention include, but are not limited to, non ionic surfactants, ionic surfactants and zwitterionic surfactants. Typical surfactants for use with the invention include, but are not limited to, sorbitan fatty acid esters (e.g. sorbitan monocaprylate, sorbitan monolaurate, sorbitan monopalmitate), sorbitan trioleate, glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearic acid amide); C10-C18 alkyl sulfates (e.g. sodium cetyl sulfate, sodium lauryl sulfate, sodium oleyl sulfate), polyoxyethylene C10-C18 alkyl ether sulfate with an average of 2 to 4 moles of ethylene oxide units added (e.g. sodium polyoxyethylene lauryl sulfate), and C1-C18 alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-C18 fatty acids. A composition may include one or more of these surfactants. Preferred surfactants are polyoxyethylene sorbitan fatty acid esters e.g. polysorbate 20, 40, 60 or 80. Polysorbate 80 (Tween 80) is particularly useful.

Suitable free amino acids for use with the invention include, but are not limited to, arginine, lysine, histidine, methionine, ornithine, isoleucine, leucine, alanine, glycine, glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g., a hydrochloride salt, such as arginine-HCl.

When present, components (i) to (iv) will be at a concentration sufficient to maintain the anti-CD40 antibody and/or the anti-C5 antibody in a form which is active and soluble after either

(i) lyophilisation and storage and reconstitution (for lyophilisates), or

(ii) conditioning in dosing units and storage (for liquid formulations).

Thus a sugar may be present in the aqueous pharmaceutical composition of the invention (or pharmaceutical compositions for use in the inventive combination of anti-CD40 antibody and/or an anti-C5 antibody), e.g. after reconstitution of a lyophilisate in water, at a concentration of between 3 and 400 mM e.g. 50-380 mM, 100-350 mM, 200-300 mM. A concentration of 270 mM sucrose is useful.

A buffering agent may be present in the aqueous pharmaceutical composition of the invention (or pharmaceutical compositions for use in the inventive combination of anti-CD40 antibody and/or an anti-C5 antibody), e.g., after reconstitution of a lyophilisate in water, at a concentration of between 1 and 60 mM e.g., 10-50 mM, 20 40 mM, 25-35 mM. A concentration of 30 mM histidine buffer is useful.

A surfactant may be present in the aqueous pharmaceutical composition of the invention (or pharmaceutical compositions for use in the inventive combination of anti-CD40 antibody and/or an anti-C5 antibody), e.g., after reconstitution of a lyophilisate in water, at a concentration of up to 0.2% (by volume) e.g., 0.01-0.1%, 0.03-0.08%, 0.04-0.08%. A concentration of 0.06% polysorbate 20 is useful. In some embodiments polysorbate 80 may be used.

A free amino acid may be present in the aqueous pharmaceutical composition of the invention (or pharmaceutical compositions for use in the inventive combination of anti-CD40 antibody and/or an anti-C5 antibody), e.g., after reconstitution of a lyophilisate in water, at a concentration of between 2 and 100 mM e.g., 10-80 mM, 20 70 mM, 30-60 mM, 40-60 mM. A concentration of 51 mM arginine (e.g., arginine-HCl) or 60 mM methionine or glycine (e.g., glycine-HCl) is useful.

In one embodiment the aqueous pharmaceutical composition consists of 150 mg/ml CFZ533 and/or 150 mg/ml tesidolumab, 30 mM histidine, 270 mM sucrose and 0.06% polysorbate 20.

In one embodiment the aqueous pharmaceutical composition consists of 150 mg/ml CFZ533 and/or 150 mg/ml tesidolumab, 30 mM histidine, 270 mM sucrose, 0.06% polysorbate 20 and 51 mM arginine-HCl.

In one embodiment the aqueous pharmaceutical composition consists of 150 mg/ml CFZ533 and/or 150 mg/ml tesidolumab, 30 mM histidine, 270 mM sucrose, 0.06% polysorbate 20 and 60 mM glycine-HCl.

In one embodiment the aqueous pharmaceutical composition consists of 200 mg/ml CFZ533 and/or 150 mg/ml tesidolumab, 30 mM histidine, 270 mM sucrose, and 0.06% polysorbate 20. In one embodiment the aqueous pharmaceutical composition consists of 200 mg/ml CFZ533 and/or 150 mg/ml tesidolumab, 30 mM histidine, 270 mM sucrose, 0.06% polysorbate 20 and 51 mM arginine-HCl.

In one embodiment the aqueous pharmaceutical composition consists of 75 mg/ml CFZ533 and/or 150 mg/ml tesidolumab, 30 mM histidine, 270 mM sucrose, and 0.06% polysorbate 20. In one embodiment the aqueous pharmaceutical composition consists of 75 mg/ml CFZ533 and/or 150 mg/ml tesidolumab, 30 mM histidine, 270 mM sucrose, 0.06% polysorbate 20 and 51 mM arginine-HCl.

Other contemplated excipients, which may be utilised in the aqueous pharmaceutical compositions of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4th edition, Rowe et al., Eds., American Pharmaceuticals Association (2003); and Remington: the Science and Practice of Pharmacy, 21th edition, Gennaro, Ed., Lippincott Williams & Wilkins (2005).

Lyophilisates

Techniques for lyophilisation of antibodies are well known in the art e.g., see John F. Carpenter and Michael J. Pikal, 1997 (Pharm. Res. 14, 969-975); Xialin (Charlie) Tang and Michael J. Pikal, 2004 (Pharm. Res. 21, 191-200). For example, the monoclonal antibody products SYNAGIS™, REMICADE™, RAPTIVA™, SIMULECT™, XOLAIR™ and HERCEPTIN™ are supplied as lyophilisates. These antibodies are reconstituted to various final concentrations e.g., SIMULECT™ is reconstituted to a concentration of 4 mg/ml antibody, REMICADE™ is reconstituted to a concentration of 10 mg/ml, HERCEPTIN™ to 21 mg/ml, SYNAGIS™ and RAPTIVA™ to 100 mg/ml, and XOLAIR™ to 125 mg/ml.

Pre-Lyophilisates, Lyophilisates and Aqueous Reconstitution

Before a lyophilisate can be administered to a patient it should be reconstituted with an aqueous reconstituent. This step permits antibody and other components in the lyophilisate to re-dissolve to give a solution which is suitable for injection to a patient.

The volume of aqueous material used for reconstitution dictates the concentration of the antibody in a resulting pharmaceutical composition. Reconstitution with a smaller volume of reconstituent than the pre-lyophilisation volume provides a composition which is more concentrated than before lyophilisation. The reconstitution factor (volume of formulation after lyophilisation:volume of formulation before) may be from 1:0.5 to 1:6. A reconstitution factor of 1:3 is useful. As mentioned above, lyophilisates of the invention can be reconstituted to give aqueous compositions with an anti-CD40 antibody and/or anti-C5 antibody concentration of at least 50 mg/ml, 100 mg/ml, 150 mg/ml. 200 mg/ml, 250 mg/ml or 300 mg/ml, and the volume of reconstituent will be selected accordingly. If required, the reconstituted formulation can be diluted prior to administration to a patient as appropriate to deliver the intended dose.

Typical reconstituents for lyophilised antibodies include sterile water or buffer, optionally containing a preservative. If the lyophilisate includes a buffering agent then the reconstituent may include further buffering agent (which may be the same as or different from the lyophilisate's buffering agent) or it may instead include no buffering agent (e.g., WFI (water for injection), or physiological saline).

When present, components (i) to (iv) will be at a pre-lyophilisation concentration sufficient to maintain the anti-CD40 and/or anti-C5 antibody in a form which is active and soluble after storage (under normal conditions) and reconstitution. The components will also be present after reconstitution.

Thus a sugar, such as sucrose or trehalose, may be present before lyophilisation at a concentration of between 3 and 300 mM e.g. 15-200 mM, 30-150 mM, 80-100 mM. A concentration of 90 mM sucrose is useful. A buffering agent, such as histidine, may be present before lyophilisation at a concentration of between 1 and 60 mM e.g., 3-30 mM, 5 20 mM, 5-15 mM. A concentration of 10 mM histidine buffer is useful. A surfactant, such as polysorbate 80 or polysorbate 20 may be present before lyophilisation at a concentration of up to 0.2% (by volume) e.g., 0.01-0.1%, 0.01-0.08%, 0.01-0.04%. A concentration of 0.02% polysorbate 80 or polysorbate 20 is useful. A free amino acid, such as arginine, methionine or glycine, may be present before lyophilisation at a concentration of between 2 and 80 mM e.g., 3-60 mM, 3-50 mM, 6 30 mM, 10-25 mM, 15-20 mM. A concentration of 17 mM arginine-HCl or 20 mM glycine-HCl or 60mM methionine is useful. The anti-CD40 antibody and/or anti-C5 antibody is present before lyophilisation at a concentration of between 20 mg/ml and 120 mg/ml, e.g., 20 mg/ml, 30 mg/ml, 40 mg/ml, 50 mg/ml, 60 mg/ml, 66.6 mg/ml, 70 mg/ml, 80 mg/ml, 90 mg/ml, 100 mg/ml, 110 mg/ml, or 120 mg/ml. A concentration of 50 mg/ml is useful.

The pre-lyophilisate of the invention has a pH between 5.0 and 8.0, between 5.0 and 7.0, between 5.5 and 6.5. In a specific embodiment, the pre-lyophilisate of the invention has a pH of about 6.0.

In one embodiment the pre-lyophilisate of the invention has a molar ratio of sucrose:antibody of 90:1 and a molar ratio of histidine:antibody of 10:1.

In one embodiment the pre-lyophilisate of the invention has a molar ratio of sucrose:antibody of 90:1, a molar ratio of histidine:antibody of 10:1, and a molar ratio of arginine-HCl:antibody of 17:1.

In one embodiment the pre-lyophilisate of the invention has a molar ratio of sucrose:antibody of 90:1, a molar ratio of histidine:antibody of 10:1, and a molar ratio of glycine-HCl:antibody of 60:1.

A formulation containing histidine buffer, sucrose, polysorbate 20 and, optionally arginine, methionine or glycine has been shown to be suitable for lyophilisation of antibody mAb1. After reconstitution, the components of the lyophilisate may be present at a concentration of the aqueous pharmaceutical compositions as described hereinbefore.

In one specific embodiment the composition is a lyophilized formulation prepared from an aqueous formulation having a pH of 6.0 and comprising:

(i) 150 mg/mL CFZ533 and/or tesidolumab

(ii) 270 mM sucrose as a stabilizer,

(iii) 30 mM L-histidine as a buffering agent, and

(iv) 0.06% Polysorbate 20 as a surfactant.

In another specific embodiment the pharmaceutical composition is an aqueous pharmaceutical composition has a pH of 6.0 and comprising:

(i) 150 mg/mL CFZ533 and/or tesidolumab

(ii) 270 mM sucrose as a stabilizer,

(iii) 30 mM L-histidine as a buffering agent, and

(iv) 0.06% Polysorbate 20 as a surfactant.

In another specific embodiment the composition is a lyophilized or liquid formulation comprising:

(i) CFZ533 and/or tesidolumab

(ii) sucrose as a stabilizer,

(iii) L-histidine as a buffering agent, and

(iv) Polysorbate 20 as a surfactant and at least one additional active pharmaceutical ingredient selected from the group consisting of a calcineurin inhibitor (CNI) such as cyclosporine (e.g. CsA, Neoral®, Novartis) or tacrolimus (e.g. Tac, FK506, Prograf®, Astellas), a lymphocyte proliferation inhibitor such as mycophenolic acid (e.g. MPA; Myfortic®, Novartis) or mycophenolate mofetil (e.g. MMF; CellCept®, Roche) or proliferation signal inhibitor such as everolimus (e.g. Zortress®, Certican®, Novartis) or sirolimus (e.g. Rapamune®, Pfizer) or a T cell co-stimulation blocker such as belatacept (e.g. Nulojix®, BMS).

The pharmaceutical composition comprising an anti-C5 antibody, or the functional fragment thereof, and an anti-CD40 antibody, or the functional fragment thereof, may be suitable for prevention of graft rejection in solid organ transplantation, and particularly prevention of graft rejection in kidney transplantation, liver transplantation, heart transplantation, lung transplantation, pancreas transplantation, intestine transplantation or composite tissue transplantation.
ii. Methods for the prevention of xenograft rejection using a pharmaceutical composition, as described in detail in section 2 above, comprising an anti-C5 antibody, or the functional fragment thereof and an anti-CD40 antibody, or the functional fragment thereof in combination with at least a pharmaceutically acceptable excipient, carrier or diluent.

In another aspect the disclosure relates to
(ii) a pharmaceutical composition comprising an anti-CD40 antibody and an anti-C5 antibody, or functional fragments thereof, as disclosed herein above for use in the prevention of graft rejection in a subject receiving a xenograft organ transplantation and to

(ii) methods for the prevention of xenograft organ rejection, comprising administering to the human recipient of a xenograft organ an anti-C5 antibody and an anti-CD40 antibody, wherein the anti-CD40 antibody is an anti-CD40 antibody, or a functional fragment thereof, (i) with silenced ADCC activity that (ii) binds both the xenograft organ and the human CD40 and (iii) said binding inhibits CD40L induced signaling with (iv) no or low agonist activity with respect to CD40 signaling. The above-described items (i)-(iv) shall have the meaning as described in section 1. Above.

The xenograft organs that can be transplanted have already been described in previous sections above in detail in section 1 above (pages 30-32) and can be an islet, a heart, a kidney, a cornea, skin, a liver or a lung. In one embodiment of the disclosure the pharmaceutical composition of the disclosure comprising an anti-CD40 antibody (e.g., CFZ533) and an anti-C5 antibody (e.g. tesidolumab), of functional fragments thereof, is used in combination with a kidney xenograft organ.

In one embodiment of the disclosure the pharmaceutical compositions comprising anti-CD40 antibody and an anti-C5 antibody as disclosed herein above (i) for use in the prevention of graft rejection or (ii) used in the method of suppressing the rejection and prolonging the survival of a xenograft organ from an animal in a human recipient is applied to a subject receiving a pig xenograft organ. In such a situation the anti-CD40 antibody, or a functional fragment thereof, comprised in the used pharmaceutical composition/method has a silenced ADCC activity, inhibits CD40L induced signaling with no or low agonist activity with respect to CD40 signaling and binds the pig CD40 and the human CD40. The xenograft pig donor organisms can be a transgenic organism as described above in section 1 above (e.g. a transgenic donor pig has been genetically modified by disrupting the a(1,3)-galactosyltransferase and cytidine monophosphate-N-acetylneuraminic acid hydroxylase (CMAH) genes or the following transgenic animals: GTKO/β4GalNT2-KO pigs; GTKO/hCD55 pigs; GTKO/hCD46/hCD55/hEPCR/hTFPI/hCD47 pigs; GTKO/β4GalNT2-KO pigs; GTKO/hCD47 pigs; GTKO pigs; GTKO/β4GalNT2-KO/hCD46/hCD47/hEPCR/hTBM/hHO-1 pigs (see section 1 above pages 30-32).

In an embodiment of the present disclosure, the pharmaceutical composition comprising an anti-CD40 antibody (e.g. CFZ533) and an anti-C5 antibody (e.g. tesidolumab)—as described above—for use in the prevention of xenograft organ (e.g. a pig organ, like a pig kidney) rejection (or for use in the method of suppressing the rejection/prolonging the survival of a xenograft organ) in a subject, is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 30 mg anti-CD40 antibody per kg of the subject, followed by a maintenance dose which is between 300 mg and 600 mg. The pharmaceutical composition comprising the anti-CD40 antibody (e.g., CFZ533) and the anti-C5 antibody, is administered to the subject at a loading dose, e.g. before, at the time of or after the xenotransplant, e.g. up to 12 hours, up to 10 hours, up to 8 hours, up to 6 hours up to 4 hours, up to 2 hours or up to one hour prior to xenotransplantation, at the time of xenotransplantation or up to 1 hour, up to 2 hours, up to 4 hours, up to 6 hours, up to 8 hours, up to 10 hours or up to 12 hours post xenotransplantation.

The loading dose of an anti-CD40 antibody (e.g., CFZ533) and an anti-C5 antibody (e.g. tesidolumab), or a functional fragments thereof may be between about 5-100 mg/kg, between about 10-50 mg/kg, may be about 10 mg/kg, about 20 mg/kg, about 30 mg/kg or about 40 mg/kg per antibody or functional fragments thereof. In certain embodiments, the loading dose for both antibodies is 30 mg/kg. In some embodiments, the loading dose for both antibodies is administered once or 2, 3, 4, 5, 6 or more times, 1 to 3, 1 to 4, 2 to 4, 2 to 5, 2 to 6, 3 to 6, 4 to 6 or 6 to 8 times. In some embodiments, the loading dose for both antibodies is administered 1, 2, 3, 4, 5, 6 or more times on one day, over 1 to 3 days, 3 to 5 days, 5 to 7 days, 5 to 10 days, 7 to 12 days, 7 to 14 days, 7 to 21 days or 14 to 21 days. In certain embodiments, the loading dose for both antibodies is administered once on the day of xenotransplantation.

The loading dose of the anti-CD40 antibody (e.g., CFZ533) and the anti-C5 antibody (e.g., tesidolumab), or a functional fragments thereof, may be administered prior to the administration of a maintenance dose. In some embodiments, the loading dose is 1.2, 1.25, 1.3, 1.35, 1.4, 1.45, 1.5, 2, 2.5, 3, 3.5, 4, 4.5, 5, 5.5, or 6 times higher than the maintenance dose, or 1.2 to 2, 2 to 3, 2 to 4, 2 to 6, 3 to 4, 3 to 6, or 4 to 6 times higher than the maintenance dose. In one embodiment, the loading dose is three times higher than the maintenance dose.

In an embodiment of the present disclosure, the pharmaceutical composition comprising an anti-CD40 antibody (e.g., CFZ533) and the anti-C5 antibody (e.g. tesidolumab), or a functional fragments thereof,—for use in the prevention of xenograft rejection (or for use in the method of suppressing the rejection/prolonging the survival of a xenograft organ) is administered to the subject as a maintenance dose. The maintenance dose is comprised of between 1 mg/kg and 50 mg/kg, between 5 mg/kg and 30 mg/kg, between 8 mg/kg and 20 mg/kg, or is about 10 mg/kg.

In certain embodiments, the maintenance dose is administered once or 2, 3, 4, 5, 6 or more times, or from 1 to 3, 1 to 4, 2 to 4, 2 to 5, 2 to 6, 3 to 6, 4 to 6, 6 to 8, or more times.

In some embodiments, the maintenance dose is administered at least twice a week, weekly, at least every two weeks, at least monthly.

The period during which the maintenance dose is administered to the subject is herein referred to as the maintenance period. During the maintenance period, the maintenance dose can be supplemented by at least one supplemental dose, as described herein below. The maintenance period can start prior to transplantation, on the day of transplantation or after the transplantation, e.g., one week, two weeks or one month after the xenotransplantation. The duration of administration of the maintenance dose, e.g., duration of the maintenance period, is at least 2 weeks, at least 4 weeks, at least 6 weeks, at least 8 weeks, at least 10 weeks, at least 3 months, at least 6 months, at least 9 months, at least one year or can be lifelong. The maintenance period can last until the xenotransplant recipient needs a new transplant.

In some embodiments, the anti-CD40 antibody (e.g., CFZ533) and the anti-C5 antibody (e.g. tesidolumab), or a functional fragments thereof, comprised in the pharmaceutical composition for use in the prevention of xenograft rejection (or for use in the method of suppressing the rejection/prolonging the survival of a xenograft organ), e.g. iscalimab or tesidolumab, are administered in such a way that a constant serum trough level of said antibodies or an antigen binding fragments thereof is achieved.

In some embodiments, the anti-CD40 antibody (e.g., CFZ533) and the anti-C5 antibody (e.g., tesidolumab), or a functional fragments thereof, comprised in the pharmaceutical composition for use in the prevention of xenograft rejection, e.g. iscalimab, or an antigen binding/functional fragment thereof (or for use in the method of suppressing the rejection/prolonging the survival of a xenograft organ), are administered in such a way that a constant serum trough level of said antibodies or antigen binding fragments thereof of 30-100 pg/mL is maintained, such as 40-100 μg/mL, 50-100 μg/mL, 55-100 μg/mL or about 50-60 μg/mL.

In other embodiments, the anti-CD40 antibody (e.g., CFZ533) and the anti-C5 antibody (e.g., tesidolumab), or a functional fragments thereof, comprised in the pharmaceutical composition for use in the prevention of xenograft rejection, e.g. iscalimab, or an antigen binding/functional fragment thereof (or for use in the method of suppressing the rejection/prolonging the survival of a xenograft organ), are administered in such a way that a constant serum trough concentration of at least 30 μg/mL, at least 40 μg/mL, at least 50 μg/mL, at least 55 μg/mL, at least 100 μg/mL, or at least 200 μg/mL per antibody is achieved.

In some embodiments, the dose of the anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ), may be increased if the trough concentration (e.g. in serum) of each of said antibodies or only one of them in the subject is below 30 μg/mL, below 40 μg/mL, below 50 μg/mL, below 60 μg/mL, below 70 μg/mL, below 80 μg/mL, below 90 μg/mL or below 100 μg/mL.

In some embodiments, the dose of the anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ) is decreased if the trough concentration (e.g. in serum) of each of the antibodies or only one of them from the subject is above 50 μg/mL, above 55 μg/mL, above 100 μg/mL, above 150 μg/mL, above 200 μg/mL, above 300 μg/mL, above 400 μg/mL or above 500 μg/mL per antibody.

In some embodiments, the dose of the anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ) is maintained if the trough concentration (e.g. in serum) of each of the antibodies or only one of them from the subject is 30-100 μg/mL, 50-100 μg/mL or 55-100 μg/mL.

According to the present disclosure, the anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ), are administered to a subject at the maintenance dose at least weekly, or at least every two weeks or at least monthly. The maintenance dose can be administered over a period of at least 6 weeks, at least 8 weeks, at least 10 weeks, at least 3 months, at least 6 months, at least 9 months, at least one year or lifelong.

In one embodiment, the anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ) are administered to a subject during a weekly maintenance period at a dose of about 10 mg/kg each. The period during which the maintenance dose is administered lasts for a period of at least 10 weeks.

According to the disclosure, anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ) are provided at a dose such that the concentrations of serum of said antibodies, e.g. constant serum trough level at steady-state of the antibodies, e.g. constant serum trough level at steady-state of total antibodies, is comprised between 30 and 100 μg/mL, 50 and 100 μg/mL, 55 to 100 ∞g/mL, 40 to 60 μg/mL or 45 to 55 μg/mL per antibody. For example, the concentration of total serum antibodies, e.g., constant serum trough level at steady-state of total antibody, is about 100 μg/mL, about 60 μg/mL or about 50 μg/mL per anti-CD40 antibody and the anti-C5 antibody.

According to the present disclosure, anti-CD40 antibody (e.g., CFZ533) and the anti-C5 antibody (e.g., tesidolumab), or functional fragments thereof, comprised in the pharmaceutical composition for use in the prevention of xenograft rejection (or for use in the method of suppressing the rejection/prolonging the survival of a xenograft organ), are administered repeatedly as defined herein.

According to the disclosure, the interval between two consecutive administrations (e.g., of maintenance dose) may vary during the treatment, e.g. may be of 1 week or two weeks, and then may increase, e.g. may double, may then be of 2 weeks or one month.

To maximize efficacy and minimize adverse effects, current immunosuppressant (IS) regimens use combinations of IS drugs as described in detail above. Hence, in one embodiment of the disclosure the pharmaceutical composition comprising anti-CD40 antibody (e.g. CFZ533) and the anti-C5 antibody (e.g. tesidolumab), or functional fragments thereof, comprised in the pharmaceutical composition for use in the prevention of xenograft rejection (or for use in the method of suppressing the rejection/prolonging the survival of a xenograft organ) is administered to a subject that has received an induction therapy prior to receiving the xenotransplant, such an induction therapy could comprise the administration of e.g. an anti-CD4 antibody and/or an anti-CD20 antibody.

In one embodiment of the disclosure the pharmaceutical composition comprising an anti-CD40 antibody (e.g. CFZ533) and the anti-C5 antibody (e.g. tesidolumab), or functional fragments thereof, comprised in the pharmaceutical composition for use in the prevention of xenograft rejection (or for use in the method of suppressing the rejection/prolonging the survival of a xenograft organ) in a subject, is combined with other anti-proliferative agents like mycophenolate mofetil (MMF), or steroids, like prednisone or T cell immunosuppressive compounds, like calcineurin inhibitors such as cyclosporin and tacrolimus.

In one embodiment of the disclosure (i) the pharmaceutical composition for use in the prevention of xenograft organ loss in solid organ transplantation patients comprises an anti CD40 antibody and an anti-C5 antibody, as described above or (ii) the method of suppressing the rejection/prolonging the survival of a xenograft organ comprising administering to the human recipient an anti CD40 antibody and an anti-C5 antibody, as described above—is used in combination with two or more of the following agents: a calcineurin inhibitor (CNI) such as cyclosporine (CsA, Neoral®, Novartis) or tacrolimus (Tac, FK506, Prograf®, Astellas), a lymphocyte proliferation inhibitor such as mycophenolic acid (MPA; Myfortic®, Novartis) or mycophenolate mofetil (MMF; CellCept®, Roche) or proliferation signal inhibitor such as everolimus (Zortress®, Certican®, Novartis) or sirolimus (Rapamune®, Pfizer) or a T cell co-stimulation blocker such as belatacept (Nulojix®, BMS). In one embodiment of the disclosure (i) the pharmaceutical composition for use in the prevention of xenograft organ loss in solid organ transplantation patients comprising an anti CD40 antibody and an anti-C5 antibody—as described above or (ii) the method of suppressing the rejection/prolonging the survival of a xenograft organ comprising administering to the human recipient an anti CD40 antibody and an anti-C5 antibody—as described above, is used in combination with a T cell co-stimulation blocker such as belatacept (Nulojix®, BMS) in a calcineurin-free treatment regimen. In one embodiment of the disclosure (i) the pharmaceutical composition for use in the prevention of xenograft organ loss in solid organ transplantation patients comprising an anti CD40 antibody and an anti-C5 antibody—as described above or (ii) the method of suppressing the rejection/prolonging the survival of a xenograft organ comprising administering to the human recipient an anti CD40 antibody and an anti-C5 antibody—as described above, is used in combination with CsA, (Neoral®, Novartis), tacrolimus (Tac, FK506, Prograf®, Astellas) and/or a mTor inhibitor such as everolimus (Zortress®, Certican®, Novartis).

### Other Immunosuppressive Therapies

In another embodiment, the herein described methods, combination therapies and uses for the prevention of xenograft loss in solid organ transplantation patients comprising administration of an anti-C5 antibody or antigen binding/functional fragment thereof, e.g. tesidolumab, eculizumab and an anti-CD40 antibody, e.g. iscalimab are applied in combination with one or more additional therapy, e.g. in combination with agents that cause T cell depletion and/or suppression, anti-proliferative agents and steroids.

The most commonly used T cell depleting agent used clinically is Thymoglobulin (rATG), an anti-thymocyte globulin, which in human patients provides profound CD4 and CD8 T Cell depression that lasts for more than 6 months with recovery to pre-existing levels by 1 year.

T cell immunosuppression can be achieved by the use of calcineurin Inhibitors. Cyclosporin and now Tacrolimus are used world-wide as the first-line maintenance for T Cell directed immunosuppression for transplant recipients. Dosing of Tacrolimus is titrated and monitored by testing for the drug in the recipient's blood. The known effective trough level of 8-12 ng/mL provides equivalent immune suppression in humans and Rhesus macaques (Fechner, J H et al., (2006), Transplantation Reviews, 20(3): 131-38). Anti-Proliferative Agents include Mycophenolate mofetil (MMF; CellCept, Roche Laboratories Inc, Nutly, N.J.), is an immunosuppressant drug used to prevent rejection in organ transplantation. It inhibits an enzyme needed for the growth of T cells and B cells. Other variations include Myfortic (mycophenolate sodium) and the active ingredient, Mycophenolic acid, is frequently used in non-human primate research. Dosing, activity and side effects are generally similar for humans and non-human primates (including the Rhesus macaque).

Steroids include corticosteroids delivered intra-venous (IV) as Methylprednisolone or orally as Prednisone have been used in transplantation since the 1960′s. IV Bolus therapy is typically used in the perioperative period followed by a descending oral dose given post-operatively and a daily dose of between 5 and 20 mg daily thereafter. A similar cycle of steroids is given when signs of rejection appear in a transplant recipient. Steroids have been found useful for blunting the cytokine release syndrome that accompanies T-Cell depleting agents and activity is similar in humans and Rhesus macaques at an equivalent mg/kg dosage (Fechner J H et al., supra).

The immunosuppressive agents as described above can be administered as single agents or in combination e.g. a triple therapy of e.g. cyclosporine (or tacrolimus) and mycophenolate mofetil (MMF) (or myfortic) and corticosteroids.

In one embodiment of the inventive method, use, combination or combination therapy, the patient receiving the xenotransplant will be receive an induction therapy with anti-CD4 and anti-CD20, will be treated with MMF and steroids and will receive the anti-C5 antibody and the anti-CD40 antibody using an inventive combination or pharmaceutical composition as disclosed herein.

4. A Combination of an Anti-C5 Antibody, or a Functional Fragment Thereof, and an Anti-CD40 Antibody, or a Functional Fragment Thereof, for Use in the Prevention of Graft Rejection in a Subject Receiving a Xenotransplant.

In another aspect the disclosure relates to

(i) combination of an anti-CD40 antibody, or a functional fragment thereof, and an anti-C5 antibody, or a functional fragment thereof, as disclosed herein above for use in the prevention of organ graft rejection in a subject receiving a xenograft organ transplantation and to

(ii) a method of suppressing the rejection and prolonging the survival of a xenograft organ from an animal in a human recipient, the method comprising administering to the human recipient a combination of an anti-CD40 antibody, or a functional fragment thereof, and an anti-C5 antibody, or a functional fragment thereof, as disclosed herein above, wherein the anti-CD40 antibody is an anti-CD40 antibody, or a functional fragment thereof, (i) with silenced ADCC activity that (ii) binds both the xenograft organ and the human CD40 and (iii) said binding inhibits CD40L induced signaling with (iv) no or low agonist activity with respect to CD40 signaling. The above-described items (i)-(iv) shall have the meaning as described herein above in previous sections.

The above-described combination (i) and the method (ii) can also be considered to be a combination therapy. Thus, one aspect of the disclosure relates to a combination therapy comprising the combination of an anti-CD40 antibody, or a functional fragment thereof, and an anti-C5 antibody, or a functional fragment thereof, as disclosed herein above, for use in the prevention of graft rejection in a subject receiving a xenograft organ.

In one aspect the disclosure relates to the above-described combination of an anti-CD40 antibody, or a functional fragment thereof, and an anti-C5 antibody, or a functional fragment thereof, wherein the antibodies are co-administered using a fixed combination of the antibodies, e.g. using the pharmaceutical composition described in detail above in section 2 above comprising said antibodies or functional fragments thereof in combination with at least a pharmaceutically acceptable excipient, carrier or diluent. In another embodiment said antibodies, in particular those described above in the sections 1-3, are administered in parallel or sequentially using two different pharmaceutical compositions comprising each only one of the two antibodies (described in detail above in section 2 above).

In one embodiment of the present disclosure, the combination of an anti-CD40 antibody and an anti-C5 antibody, or functional fragments thereof, can be administered as described above in detail in section 2 through a loading dose and/or a maintenance dose. Such a loading dosing can consist of one, two, three or four intravenous administration(s) of a first dose and such a maintenance dosing can consist of weekly or biweekly subcutaneous injections of a second dose. Such a first dose is at least 10 mg and up to 30 mg of the anti-CD40 antibody or functional fragment thereof and the anti-C5 antibody or functional fragments thereof per kg of the subject, followed by a maintenance dose of said antibody combination which is between 300 mg and 600 mg per antibody. The antibody combination described above is administered to the subject at a loading dose, e.g. before, at the time of or after the xenotransplant, e.g. up to 12 hours, up to 10 hours, up to 8 hours, up to 6 hours up to 4 hours, up to 2 hours or up to one hour prior to xenotransplantation, at the time of xenotransplantation or up to 1 hour, up to 2 hours, up to 4 hours, up to 6 hours, up to 8 hours, up to 10 hours or up to 12 hours post xenotransplantation.

The loading dose of the anti-CD40 antibody (e.g., CFZ533 or functional fragments thereof) and anti-C5 antibody (e.g., tesidolumab or functional fragments thereof) combination may be between about 5-100 mg/kg, between about 10-50 mg/kg, may be about 10 mg/kg, about 20 mg/kg, about 30 mg/kg or about 40 mg/kg per antibody or functional fragments thereof. In certain embodiments, the loading dose for both antibodies is 30 mg/kg.

In some embodiments, the anti-CD40 antibody (e.g., CFZ533) and the anti-C5 antibody (e.g. tesidolumab), or a functional fragments thereof, are combined for use in the prevention of xenograft rejection in such a way that a constant serum trough level of said antibodies or an antigen binding fragments thereof is achieved, as described in section 2 above in detail.

According to the present disclosure, the anti-CD40 antibody (e.g., CFZ533) and the anti-C5 antibody (e.g., tesidolumab), or functional fragments thereof, are combined for use in the prevention of xenograft rejection, in such a way that they are administered to a subject at the maintenance dose at least weekly, or at least every two weeks or at least monthly. The maintenance dose can be administered over a period of at least 6 weeks, at least 8 weeks, at least 10 weeks, at least 3 months, at least 6 months, at least 9 months, at least one year or lifelong.

In one embodiment of the disclosure the anti-CD40 antibody (e.g., CFZ533) and the anti-C5 antibody (e.g., tesidolumab), or functional fragments thereof, are combined for use in the prevention of xenograft rejection in such a way that they are administered—as described above—to a subject with a different time schedule. Such a different time schedule could comprise a first administration of an anti-C5 antibody for a certain period of time (e.g., hours, days or weeks) followed by a period of a parallel or combined administration of an anti-C5 and anti-CD40 antibody (e.g., hours or days or weeks), followed by a period wherein only the anti-CD40 antibody is administered to the patients. In one embodiment, the above-described schedule will start with an anti CD40 antibody administration, followed by an overlapping anti-C5 antibody/anti-CD40 antibody administration, followed by an anti-C5 administration.

In a preferred embodiment the formulations described above in section 2 will be used to administer the anti-C5 antibody/anti-CD40 antibody using separate formulations.

In one embodiment the combination of an anti-C5 antibody, or a functional fragment thereof, and an anti-CD40 antibody, or a functional fragment thereof, for use in the prevention of graft rejection in a subject receiving a xenotransplant is administered to a patient having received a pig organ as described in detail above in the previous section. The use of the described transgenic donor pigs is a particular embodiment of the invention.

5. Use of an Anti-C5 Antibody and an Anti-CD40 Antibody, or Functional Fragments Thereof, in the Manufacture of a Medicament for the Prevention of Graft Rejection in a Subject Receiving a Xenograft.

Disclosed herein is the use of an anti-C5 antibody and an anti-CD40 antibody, or functional fragments thereof, for the manufacture of a medicament for the prevention of graft rejection in a subject receiving a xenograft, wherein the medicament is formulated to comprise containers, each container having a sufficient amount of the anti-C5 antibody and the anti-CD40 antibody to allow delivery of at least about 75 mg, 150 mg, 300 mg or 600 mg of the anti-C5 antibody and the anti-CD40 antibody or antigen binding/functional fragments thereof per unit dose.

Also disclosed herein is the use of anti-C5 antibody and the anti-CD40 antibody or antigen binding fragments thereof for the manufacture of a medicament for the prevention of graft rejection in solid organ transplantation in a subject receiving a xenograft, wherein the medicament is formulated at a dosage to allow systemic delivery (e.g., intravenous or subcutaneous delivery) of 75 mg, 150 mg, 300 mg of 600 mg anti-C5 antibody and the anti-CD40 antibody or antigen binding/functional fragments thereof per unit dose.

In one embodiment, the anti-CD40 antibody used for the manufacture of a medicament for the prevention of graft rejection in a subject receiving a xenograft as describe above in combination with an anti-C5 antibody is selected from the group consisting of:
a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34;
d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36; and
e. iscalimab, and

the anti-C5 antibody used for the manufacture of a medicament for the prevention of graft rejection in a subject receiving a xenograft as describe above in combination with the above described anti-CD40 antibodies a) to e) is selected from the group consisting of:
a. an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
b. an anti-C5 antibody comprising an immunoglobulin VH domain comprising the amino acid sequence of SEQ ID NO: 7 and an immunoglobulin VL domain comprising the amino acid sequence of SEQ ID NO: 8;
c. an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
d. an anti-C5 antibody comprising an immunoglobulin VH domain comprising the amino acid sequence of SEQ ID NO: 17 and an immunoglobulin VL domain comprising the amino acid sequence of SEQ ID NO: 18; and
e. tesidolumab or eculizumab.

6. Kits of Part

The disclosure also encompasses kits for treating a transplantation patient having received a xenotransplant as described above (as the case may be) with an anti-C5 antibody and an anti-CD40 antibody or antigen binding/functional fragments thereof. Such a kit is particularly suitable in connections with the inventive combination of an anti-C5 antibody and the anti-CD40 antibody or antigen binding fragments thereof described herein. Such kits comprise anti-C5 antibody and an anti-CD40 antibody or antigen binding/functional fragments thereof (e.g., in liquid or lyophilized form) comprised in one pharmaceutical composition comprising both antibodies or two pharmaceutical composition comprising one of the two antibodies each (described supra). Additionally, such kits may comprise means for administering the antibodies (e.g., a syringe and vial, a prefilled syringe, a prefilled pen, a patch/pump) and instructions for use. The instructions may disclose providing the anti-CD40 antibody and the anti-C5 antibody to the patient as part of a specific dosing regimen.

In one embodiment, the means for administering, such as an autoinjector, are part of a system comprising means for detecting and processing plasma concentration of drug in real-time. In a preferred embodiment, the system comprises means to compare the plasma concentration of an anti-CD40 antibody and the anti-C5 antibody with a threshold value and adjust the dose accordingly.

Disclosed herein are kits for the treatment of a transplantation patient, comprising: a) a pharmaceutical composition comprising a therapeutically effective amount of an anti-CD40 antibody or antigen binding/functional fragment thereof; b) a therapeutically effective amount of an anti-C5 antibody or antigen binding/functional fragment thereof; c) means for administering the anti-CD40 antibody and the C5 antibody or antigen binding fragments thereof to the patient; and d) instructions providing administration of the anti-CD40/anti-C5 antibody or antigen binding fragments thereof to a patient in need thereof at a dose of about 3 to about 30 mg active ingredient per kilogram of a human subject (on multiple occasions).

In one specific embodiment, a use is provided, of a) a liquid pharmaceutical composition comprising an anti-CD40 antibody and/or an anti-C5 antibody (or functional fragments thereof), a buffer, a stabilizer and a solubilizer, and b) means for subcutaneously administering the antibodies to a transplantation patient, for the manufacture of a medicament for the prevention of graft rejection in solid organ transplantation, wherein the antibodies or functional fragments thereof:

i) are to be subcutaneously administered to the patient with a dose of about 3 to about 30 mg, such as 10 mg, active ingredient per kilogram of a human subject, three times, once every other week; and

ii) thereafter, is to be subcutaneously administered to the patient as monthly doses of about 3 to about 30 mg, such as 10 mg, active ingredient per kilogram of a human subject, wherein said antibodies are selected from the groups consisting of:
a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34;
d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36; and e. iscalimab, and
e. an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
f. an anti-C5 antibody comprising an immunoglobulin VH domain comprising the amino acid sequence of SEQ ID NO: 7 and an immunoglobulin VL domain comprising the amino acid sequence of SEQ ID NO: 8;
g. an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
h. an anti-C5 antibody comprising an immunoglobulin VH domain comprising the amino acid sequence of SEQ ID NO: 17 and an immunoglobulin VL domain comprising the amino acid sequence of SEQ ID NO: 18; and
i. tesidolumab or eculizumab.

In one embodiment, the invention provides a kit comprising two separate pharmaceutical compositions which contain an anti-CD40 antibody and an anti-C5 antibody (or functional fragments thereof), respectively. In one embodiment, the kit comprises means for separately retaining said compositions, such as a container or divided bottle, or divided foil packet. The kit of the invention may be used for administering different dosage forms, for example, intravenous or subcutaneous, for administering the separate compositions at different dosage intervals, or for titrating the separate compositions against one another. To assist compliance, the kit of the invention typically comprises directions for administration.

In the combination therapies of the invention, the anti-CD40 antibody and an anti-C5 antibody may be manufactured and/or formulated by the same or different manufacturers.

### Numbered Embodiments

While various specific embodiments have been illustrated and described, it will be appreciated that various changes can be made without departing from the spirit and scope of the disclosure(s). The present disclosure is exemplified by the numbered embodiments set forth below.
1. A Pharmaceutical composition comprising an anti-CD40 antibody for use in the prevention of graft rejection in a subject receiving a xenograft organ, wherein the anti-CD40 antibody is an anti-CD40 antibody, or a functional fragment thereof, with silenced ADCC activity that binds both the xenograft organ and the human CD40 and said binding inhibits CD40L induced signaling with no or low agonist activity with respect to CD40 signaling.
2. Pharmaceutical composition for use according to embodiment 1, wherein the xenograft organ is from a pig and the anti-CD40 antibody binds the pig CD40.
3. Pharmaceutical composition for use according to embodiment 2, wherein the pig is a transgenic organism.
4. Pharmaceutical composition for use according to embodiment 3, wherein the transgenic pig has been genetically modified as follows: disrupted a(1,3)-galactosyltransferase and cytidine monophosphate-N-acetylneuraminic acid hydroxylase (CMAH) genes.
5. Pharmaceutical composition for use according to embodiments 1-4, wherein the anti-CD40 antibody or functional fragment thereof is selected from the group consisting of:
   a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
   b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
   c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34;
   d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36; and.
   e. an anti-CD40 antibody, wherein the anti-CD40 antibody is iscalimab.
6. Pharmaceutical composition for use according to embodiments 1-5, wherein the anti-CD40 antibody or functional fragment thereof is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 30 mg anti-CD40 antibody per kg of the subject, followed by a maintenance dose which is between 300 mg and 600 mg.
7. Pharmaceutical composition for use according to embodiment 6, wherein the loading dose of the anti-CD40 antibody or functional fragment thereof is administered at a dose of about 10 mg/kg to about 50 mg/kg per antibody.
8. Pharmaceutical composition for use according to embodiment 7, wherein the loading dose of the anti-CD40 antibody or functional fragment thereof is administered as a single dose of about 10 mg/kg on the day of xenograft transplantation.
9. Pharmaceutical composition for use according to embodiments 1-8, wherein the subject is administered an induction therapy prior to receiving the xenotransplant.
10. Pharmaceutical composition for use according to embodiment 9, wherein the induction therapy is administration of an anti-CD4 antibody and/or an anti-CD20 antibody.
11. Pharmaceutical composition for use according to embodiments 1-10, wherein the anti-CD40 antibody or functional fragment thereof is administered in combination with other anti-proliferative agents like mycophenolate mofetil (MMF), or steroids, like prednisone or T cell immunosuppressive compounds, like calcineurin inhibitors such as cyclosporin and tacrolimus.
12. A method of suppressing the rejection of a xenograft donor organ from an animal in a human recipient, the method comprising administering to the human recipient an anti-CD40 antibody, wherein the anti-CD40 antibody is an anti-CD40 antibody, or a functional fragment thereof, with silenced ADCC activity that binds both the xenograft and the human CD40 and said binding inhibits CD40L induced signaling with no or low agonist activity with respect to CD40 signaling.
13. Method according to embodiment 12, wherein the xenograft donor organ is a pig organ and the anti-CD40 antibody or functional fragment thereof binds the pig CD40.
14. Method according to embodiment 13, wherein the pig is a transgenic organism.
15. Method according to embodiment 14, wherein the transgenic pig has been genetically modified and has disrupted a(1,3)-galactosyltransferase and CMAH genes.
16. Method according to according to embodiments 12-15, wherein the anti-CD40 antibody or functional fragment thereof is selected from the group consisting of:
   a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
   b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
   c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34;
   d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36; and
   e. an anti-CD40 antibody, wherein the anti-CD40 antibody is iscalimab.
17. Method according to according to embodiments 12-16, wherein the anti-CD40 antibody or functional fragment thereof is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 30 mg anti-CD40 antibody or functional fragment thereof per kg of the recipient, followed by a maintenance dose which is between 300 mg and 600 mg.
18. Method according to embodiment 17, wherein the loading dose of the anti-CD40 antibody or functional fragment thereof is administered at a dose of about 10 mg/kg to about 50 mg/kg per antibody.
19. Method according to embodiment 18, wherein the loading dose of the anti-CD40 antibody or functional fragment thereof is administered as a single dose of about 10 mg/kg on the day of xenograft transplantation.
20. Method according to embodiments 12-19, wherein the recipient is administered an induction therapy prior to receiving the xenotransplant.
21. Method according to embodiment 20, wherein the induction therapy is administration of an anti-CD4 antibody and/or an anti-CD20 antibody.
22. Method according to embodiments 12-21, wherein the anti-CD40 antibody treatment is combined with other anti-proliferative agents like mycophenolate mofetil (MMF), or steroids, like prednisone or T cell immunosuppressive compounds, like calcineurin inhibitors such as cyclosporin and tacrolimus.
23. Pharmaceutical composition comprising an anti-C5 antibody, or a functional fragment thereof and an anti-CD40 antibody, or a functional fragment thereof, in combination with at least a pharmaceutically acceptable excipient, carrier or diluent.
24. Pharmaceutical composition of embodiment 23, wherein the anti-CD40 antibody is an anti-CD40 antibody, or a functional fragment thereof, with silenced ADCC activity that binds both the xenograft and the human CD40 and said binding inhibits CD40L induced signaling with no or low agonist activity with respect to CD40 signaling.
25. Pharmaceutical composition according to embodiment 24, wherein the anti-CD40 antibody or the functional fragment thereof is selected from the group consisting of:
   a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
   b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
   c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34; and
   d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36.
26. Pharmaceutical composition according to embodiment 25, wherein the anti-CD40 antibody is iscalimab.
27. Pharmaceutical composition according to embodiments 23-26, wherein the anti-C5 antibody is an antibody selected from the group consisting of:
   a. an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
   b. an anti-C5 antibody comprising an immunoglobulin VH domain comprising the amino acid sequence of SEQ ID NO: 7 and an immunoglobulin VL domain comprising the amino acid sequence of SEQ ID NO: 8;
   c. an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
   d. an anti-C5 antibody comprising an immunoglobulin VH domain comprising the amino acid sequence of SEQ ID NO: 17 and an immunoglobulin VL domain comprising the amino acid sequence of SEQ ID NO: 18.
28. Pharmaceutical composition according to embodiment 27, wherein the anti-C5 antibody is tesidolumab or eculizumab.
29. Pharmaceutical composition according to the embodiments 23-28 for use in the prevention of graft rejection in a subject receiving a xenograft organ.
30. Pharmaceutical composition for use according to embodiment 29, wherein the anti-CD40 antibody is an anti-CD40 antibody, or a functional fragment thereof, with silenced ADCC activity that binds both the xenograft and the human CD40 and said binding inhibits CD40L induced signaling with no or low agonist activity with respect to CD40 signaling.
31. Pharmaceutical composition for use according to according to embodiment 30, wherein the antibodies are co-administered using a composition comprising a fixed combination of the antibodies, through a loading dose and/or a maintenance dose.
32. Pharmaceutical composition for use according to embodiment 31, administered as fixed combination, wherein
   a) the loading dose of the anti-C5-antibody is administered at a dose of about 10 mg/kg to about 50 mg/kg per antibody and
   b) the loading dose of the anti-CD40 antibody is administered at a dose of about 10 mg/kg to about 50 mg/kg per antibody.
33. Pharmaceutical composition for use according to embodiment 32, wherein the loading dose of the anti-CS-antibody is administered as a single dose of about 10 mg/kg on the day of xenograft transplantation and the anti-CD40-antibody is administered as a single dose of about 10 mg/kg on the day of xenograft transplantation.
34. Pharmaceutical composition for use according to any one of the preceding embodiments, wherein the route of administration of the pharmaceutical composition is subcutaneous or intravenous.
35. Pharmaceutical composition for use according to embodiments 29-34, wherein the xenograft organ is a pig organ and the anti-CD40 antibody binds the pig CD40.
36. Pharmaceutical composition for use according to embodiment 35, wherein the pig is a transgenic organism.
37. Pharmaceutical composition for use according to embodiment 36, wherein the transgenic pig has been genetically modified as follows: disrupted a(1,3)-galactosyltransferase and CMAH genes.
38. Pharmaceutical composition for use according to embodiments 29-37, wherein the subject is administered an induction therapy prior to receiving the xenotransplant.
39. Pharmaceutical composition for use according to embodiment 38, wherein the induction therapy is administration of an anti-CD4 antibody and/or an anti-CD20 antibody.
40. Pharmaceutical composition for use according to embodiments 29-39, wherein the anti-C5 antibody and an anti-CD40 antibody treatment is combined with other anti-proliferative agents like mycophenolate mofetil (MMF), or steroids, like prednisone or T cell immunosuppressive compounds, like calcineurin inhibitors such as cyclosporin and tacrolimus.
41. A combination of an anti-C5 antibody, or a functional fragment thereof, and an anti-CD40 antibody, or a functional fragment thereof, for use in the prevention of graft rejection in a subject receiving a xenotransplant.
42. A combination for use according to embodiment 41, wherein the anti-CD40 antibody is an anti-CD40 antibody with silenced ADCC activity that binds both the xenograft and the human CD40 and said binding inhibits CD40L induced signaling with no or low agonist activity with respect to CD40 signaling.
43. A combination for use according to embodiment 42, wherein the antibodies are co-administered using a fixed combination of the antibodies, or both antibodies are administered in parallel or sequentially using two different pharmaceutical compositions comprising each only one of the two antibodies through a loading dose and/or a maintenance dose.
44. A combination for use according to embodiment 43, administered as fixed combination, in parallel or sequentially, wherein
   a) the loading dose of the anti-C5-antibody is administered at a dose of about 10 mg/kg to about 50 mg/kg per antibody and
   b) the loading dose of the anti-CD40 antibody is administered at a dose of about 10 mg/kg to about 50 mg/kg per antibody.
45. A combination for use according to embodiment 44, wherein the loading dose of the anti-CS-antibody is administered as a single dose of about 10 mg/kg on the day of xenograft transplantation and the anti-CD40-antibody is administered as a single dose of about 10 mg/kg on the day of xenograft transplantation.
46. A combination for use according to embodiments 41-45, wherein the route of administration of the anti-C5 antibody is subcutaneous or intravenous, and/or wherein the administration the anti-CD40 antibody is subcutaneous or intravenous.
47. A combination for use according to embodiments 41-46, wherein the xenograft is a pig organ and the anti-CD40 antibody binds the pig CD40.
48. A combination for use according to embodiment 47, wherein the pig is a transgenic organism.
49. A combination for use according to embodiment 48, wherein the transgenic pig has been genetically modified as follows: disrupted a(1,3)-galactosyltransferase and CMAH genes.
50. A combination for use according to embodiments 41-49, wherein the subject is administered an induction therapy prior to receiving the xenotransplant.
51. A combination for use according to embodiment 50, wherein the induction therapy is administration of an anti-CD4 antibody and/or an anti-CD20 antibody.
52. A combination for use according to embodiments 41-51, wherein the anti-CD40 antibody is an anti-CD40 antibody selected from the group consisting of:
   a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
   b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
   c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34; and
   d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36.
53. A combination for use according to embodiments 52, wherein the anti-CD40 antibody is iscalimab.
54. A combination for use according to embodiments 41-53, wherein the anti-C5 antibody is an antibody selected from the group consisting of:
   a. an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
   b. an anti-C5 antibody comprising an immunoglobulin VH domain comprising the amino acid sequence of SEQ ID NO: 7 and an immunoglobulin VL domain comprising the amino acid sequence of SEQ ID NO: 8;
   c. an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
   d. an anti-C5 antibody comprising an immunoglobulin VH domain comprising the amino acid sequence of SEQ ID NO: 17 and an immunoglobulin VL domain comprising the amino acid sequence of SEQ ID NO: 18.
55. A combination for use according to embodiments 54, wherein the anti-C5 antibody is tesidolumab or eculizumab.
56. A method of suppressing the rejection of a xenograft organ from an animal in a human recipient, the method comprising administering to the human recipient an anti-C5 antibody and an anti-CD40 antibody or functional fragments thereof.
57. A method according to embodiment 56, wherein the pharmaceutical composition of embodiments 23-28 or the combination according to embodiments 41-55 is administered to the human recipient.
58. A method according to embodiments 56-57, wherein the antibodies are co-administered using a fixed combination of the antibodies, or both antibodies are administered in parallel or sequentially using two different pharmaceutical compositions comprising each only one of the two antibodies through a loading dose and/or a maintenance dose.
59. A method according to embodiment 58, wherein the fixed combination, or parallel or sequentially administered antibodies are administered through,
   a) a loading dose of the anti-CS-antibody, or a functional fragment thereof, at a dose of about 10 mg/kg to about 50 mg/kg per antibody and
   b) a loading dose of the anti-CD40 antibody, or a functional fragment thereof, at a dose of about 10 mg/kg to about 50 mg/kg per antibody.
60. A method according to embodiments 56-59, wherein the loading dose of the anti-C5-antibody, or a functional fragment thereof, is administered as a single dose of about 10 mg/kg on the day of xenograft transplantation and the anti-CD40-antibody, or a functional fragment thereof, is administered as a single dose of about 10 mg/kg on the day of xenograft transplantation.
61. A method according to embodiments 56-60, wherein the route of administration of the anti-C5 antibody, or a functional fragment thereof, is subcutaneous or intravenous, and/or wherein the administration the anti-CD40 antibody, or a functional fragment thereof, is subcutaneous or intravenous.
62. A method according to embodiments 56-61, wherein the xenograft is a pig organ and the anti-CD40 antibody binds the pig CD40.
63. A method according to embodiment 62, wherein the pig organ is from a transgenic organism.
64. A method according to embodiment 63, wherein the transgenic pig has been genetically modified as follows: disrupted a(1,3)-galactosyltransferase and CMAH genes.
65. A method according to embodiments 56-64, wherein the recipient is administered an induction therapy prior to receiving the xenotransplant.
66. A method according to embodiment 65, wherein the induction therapy is administration of an anti-CD4 antibody and/or an anti-CD20 antibody.
67. A method according to embodiment 56-66, wherein the anti-CD40 antibody is an anti-CD40 antibody selected from the group consisting of:
   a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
   b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
   c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34; and
   d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36.
68. A method according to embodiment 67, wherein the anti-CD40 antibody is iscalimab.
69. A method according to embodiments 56-68, wherein the anti-C5 antibody is an antibody selected from the group consisting of:
   a. an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
   b. an anti-C5 antibody comprising an immunoglobulin VH domain comprising the amino acid sequence of SEQ ID NO: 7 and an immunoglobulin VL domain comprising the amino acid sequence of SEQ ID NO: 8;
   c. an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
   d. an anti-C5 antibody comprising an immunoglobulin VH domain comprising the amino acid sequence of SEQ ID NO: 17 and an immunoglobulin VL domain comprising the amino acid sequence of SEQ ID NO: 18.
70. A method according to embodiment 69, wherein the anti-C5 antibody is tesidolumab or eculizumab.
71. A method according to embodiments 56-70, wherein the anti-C5 antibody and anti-CD40 antibody treatment is combined with other anti-proliferative agents like mycophenolate mofetil (MMF), or steroids, like prednisone or T cell immunosuppressive compounds, like calcineurin inhibitors such as cyclosporin and tacrolimus.
72. Use of an anti-C5 antibody, or a functional fragment thereof, and an anti-CD40 antibody, or a functional fragment thereof, in the manufacture of a medicament for the prevention of graft rejection in a subject receiving a xenograft.
73. Use of an anti-C5 antibody, or a functional fragment thereof, and an anti-CD40 antibody, or a functional fragment thereof, according to embodiment 72, wherein the anti-CD40 antibody is an anti-CD40 antibody with silenced ADCC activity that binds both the xenograft and the human CD40 and said binding inhibits CD40L induced signaling with no or low agonist activity with respect to CD40 signaling.
74. Use of an anti-C5 antibody, or a functional fragment thereof, and an anti-CD40 antibody, or a functional fragment thereof, according to embodiments 72-73, wherein the CD40 antibody is an anti-CD40 antibody selected from the group consisting of:
   a. an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
   b. an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
   c. an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34; and
   d. an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36.
75. Use of an anti-C5 antibody and an anti-CD40 antibody according to embodiments 74, wherein the anti-CD40 antibody is iscalimab.
76. Use of an anti-C5 antibody, or a functional fragment thereof, and an anti-CD40 antibody, or a functional fragment thereof, according to embodiments 72-75, wherein the anti-C5 antibody is an antibody selected from the group consisting of:
   a. an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
   b. an anti-C5 antibody comprising an immunoglobulin VH domain comprising the amino acid sequence of SEQ ID NO: 7 and an immunoglobulin VL domain comprising the amino acid sequence of SEQ ID NO: 8;
   c. an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
   d. an anti-C5 antibody comprising an immunoglobulin VH domain comprising the amino acid sequence of SEQ ID NO: 17 and an immunoglobulin VL domain comprising the amino acid sequence of SEQ ID NO: 18.
77. Use of an anti-C5 antibody and an anti-CD40 antibody according to embodiment 76, wherein the anti-C5 antibody is tesidolumab or eculizumab.
78. A kit of parts comprising:
   (i) an anti-CD40 antibody, or a functional fragment thereof, with silenced ADCC activity that binds both the xenograft organ and the human CD40 and said binding inhibits CD40L induced signaling with no or low agonist activity with respect to CD40 signaling
   (ii) an anti-C5 antibody, or a functional fragment thereof,
   (iii) administration means
   (iv) instructions for their use
   and optionally further comprising
   (v) at least one other excipients, diluents or carriers.
79. A kit of parts according to embodiment 78, comprising an anti-CD40 antibody according to embodiment 5.
80. A kit of parts according to embodiments 78 and 79, comprising an C5 antibody according to embodiments 27 or 28.
81. A kit of parts according to embodiment 78, comprising a pharmaceutical composition according to embodiments 23-28.

TABLE 1
SEQUENCES
Sequences

| SEQ ID | | |
|---|---|---|
| NO. | Information | Sequence |
| 1 | tesidolumab | SYAIS |
| | HCDR1 (Kabat) | |
| 2 | tesidolumab | GIGPFFGTANYAQKFQG |
| | HCDR2 (Kabat) | |
| 3 | tesidolumab | DTPYFDY |
| | HCDR3 (Kabat) | |
| 4 | tesidolumab | SGDSIPNYYVY |
| | LCDR1 (Kabat) | |
| 5 | tesidolumab | DDSNRPS |
| | LCDR2 (Kabat) | |
| 6 | tesidolumab | QSFDSSLNAEV |
| | LCDR3 (Kabat) | |
| 7 | tesidolumab VH | EVQLVQSGAEVKKPGSSVKVSCKASGGTFSSYAISWVRQA |
| | | PGQGLEWMGGIGPFFGTANYAQKFQGRVTITADESTSTAY |
| | | MELSSLRSEDTAVYYCARDTPYFDYWGQGTLVTVSS |
| 8 | tesidolumab VL | SYELTQPLSVSVALGQTARITCSGDSIPNYYVYWYQQKPG |
| | | QAPVLVIYDDSNRPSGIPERFSGSNSGNTATLTISRAQAG |
| | | DEADYYCQSFDSSLNAEVFGGGTKLTVL |
| 9 | tesidolumab HC | EVQLVQSGAEVKKPGSSVKVSCKASGGTFSSYAISWVRQA |
| | | PGQGLEWMGGIGPFFGTANYAQKFQGRVTITADESTSTAY |
| | | MELSSLRSEDTAVYYCARDTPYFDYWGQGTLVTVSSASTK |
| | | GPSVFPLAPSSKSTSGGTAALGCLVKDYFPEPVTVSWNSG |
| | | ALTSGVHTFPAVLQSSGLYSLSSWTVPSSSLGTQTYICNV |
| | | NHKPSNTKVDKRVEPKSCDKTHTCPPCPAPEAAGGPSVFL |
| | | FPPKPKDTLMISRTPEVTCVVVDVSHEDPEVKFNWYVDGV |
| | | EVHNAKTKPREEQYNSTYRVVSVLTVLHQDWLNGKEYKCK |
| | | VSNKALPAPIEKTISKAKGQPREPQVYTLPPSREEMTKNQ |
| | | VSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| 10 | tesidolumab LC | SYELTQPLSVSVALGQTARITCSGDSIPNYYVYWYQQKPG |
| | | QAPVLVIYDDSNRPSGIPERFSGSNSGNTATLTISRAQAG |
| | | DEADYYCQSFDSSLNAEVFGGGTKLTVLGQPKAAPSVTLF |
| | | PPSSEELQANKATLVCLISDFYPGAVTVAWKADSSPVKAG |
| | | VETTTPSKQSNNKYAASSYLSLTPEQWKSHRSYSCQVTHE |
| | | GSTVEKTVAPTECS |
| 11 | eculizumab | NYWIQ |
| | HCDR1 | |
| 12 | eculizumab | EILPGSGSTEYTENFKD |
| | HCDR2 | |
| 13 | eculizumab | YFFGSSPNWYFDV |
| | HCDR3 | |
| 14 | eculizumab | GASENIYGALN |
| | LCDR1 | |
| 15 | eculizumab | GATNLAD |
| | LCDR2 | |
| 16 | eculizumab | QNVLNTPLT |
| | LCDR3 | |
| 17 | eculizumab VH | QVQLVQSGAEVKKPGASVKVSCKASGYIFSNYWIQWVRQA |
| | | PGQGLEWMGEILPGSGSTEYTENFKDRVTMTRDTSTSTVY |
| | | MELSSLRSEDTAVYYCARYFFGSSPNWYFDVWGQGTLVTV |
| | | SS |
| 18 | eculizumab VL | DIQMTQSPSSLSASVGDRVTITCGASENIYGALNWYQQKP |
| | | GKAPKLLIYGATNLADGVPSRFSGSGSGTDFTLTISSLQP |
| | | EDFATYYCQNVLNTPLTFGQGTKVEIK |
| 19 | eculizumab HC | QVQLVQSGAEVKKPGASVKVSCKASGYIFSNYWIQWVRQA |
| | | PGQGLEWMGEILPGSGSTEYTENFKDRVTMTRDTSTSTVY |
| | | MELSSLRSEDTAVYYCARYFFGSSPNWYFDVWGQGTLVTV |
| | | SSASTKGPSVFPLAPCSRSTSESTAALGCLVKDYFPEPVT |
| | | VSWNSGALTSGVHTFPAVLQSSGLYSLSSVVTVPSSNFGT |
| | | QTYTCNVDHKPSNTKVDKTVERKCCVECPPCPAPPVAGPS |
| | | VFLFPPKPKDTLMISRTPEVTCVVVDVSQEDPEVQFNWYV |
| | | DGVEVHNAKTKPREEQFNSTYRVVSVLTVLHQDWLNGKEY |
| | | KCKVSNKGLPSSIEKTISKAKGQPREPQVYTLPPSQEEMT |
| | | KNQVSLTCLVKGFYPSDIAVEWESNGQPENNYKTTPPVLD |
| | | SDGSFFLYSRLTVDKSRWQEGNVFSCSVMHEALHNHYTQK |
| | | SLSLSLGK |
| 20 | eculizumab LC | DIQMTQSPSSLSASVGDRVTITCGASENIYGALNWYQQKP |
| | | GKAPKLLIYGATNLADGVPSRFSGSGSGTDFTLTISSLQP |
| | | EDFATYYCQNVLNTPLTFGQGTKVEIKRTVAAPSVFIFPP |
| | | SDEQLKSGTASWCLLNNFYPREAKVQWKVDNALQSGNSQE |
| | | SVTEQDSKDSTYSLSSTLTLSKADYEKHKVYACEVTHQGL |
| | | SSPVTKSFNRGEC |
| 21 | BNJ441 HC | QVQLVQSGAEVKKPGASVKVSCKASGHIFSNYWIQWVRQA |
| | | PGQGLEWMGEILPGSGHTEYTENFKDRVTMTRDTSTSTVY |
| | | MELSSLRSEDTAVYYCARYFFGSSPNWYFDVWGQGTLVTV |
| | | SSASTKGPSVFPLAPCSRSTSESTAALGCLVKDYFPEPVT |
| | | VSWNSGALTSGVHTFPAVLQSSGLYSLSSVVTVPSSNFGT |
| | | QTYTCNVDHKPSNTKVDKTVERKCCVECPPCPAPPVAGPS |
| | | VFLFPPKPKDTLMISRTPEVTCVVVDVSQEDPEVQFNWYV |
| | | DGVEVHNAKTKPREEQFNSTYRVVSVLTVLHQDWLNGKEY |
| | | KCKVSNKGLPSSIEKTISKAKGQPREPQVYTLPPSQEEMT |
| | | KNQVSLTCLVKGFYPSDIAVEWESNGQPENNYKTTPPVLD |
| | | SDGSFFLYSRLTVDKSRWQEGNVFSCSVLHEALHSHYTQK |
| | | SLSLSLGK |
| 22 | BNJ441 LC | DIQMTQSPSSLSASVGDRVTITCGASENIYGALNWYQQKP |
| | | GKAPKLLIYGATNLADGVPSRFSGSGSGTDFTLTISSLQP |
| | | EDFATYYCQNVLNTPLTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 23 | ALXN1210 HC | QVQLVQSGAEVKKPGASVKVSCKASGHIFSNYWIQWVRQA |
| | | PGQGLEWMGEILPGSGHTEYTENFKDRVTNTRDTSTSTVY |
| | | MELSSLRSEDTAVYYCARYFFGSSPNWYFDVWGQGTLVTV |
| | | SSASTKGPSVFPLAPCSRSTSESTAALGCLVKDYFPEPVT |
| | | VSWNSGALTSGVHTFPAVLQSSGLYSLSSVVTVPSSNFGT |
| | | QTYTCNVDHKPSNTKVDKTVERKCCVECPPCPAPPVAGPS |
| | | VFLFPPKPKDTLMISRTPEVTCVVVDVSQEDPEVQFNWYV |
| | | DGVEVHNAKTKPREEQFNSTYRVVSVLTVLHQDWLNGKEY |
| | | KCKVSNKGLPSSIEKTISKAKGQPREPQVYTLPPSQEEMT |
| | | KNQVSLTCLVKGFYPSDIAVEWESNGQPENNYKTTPPVLD |
| | | SDGSFFLYSRLTVDKSRWQEGNVFSCSVLHEALHSHYTQK |
| | | SLSLSLGK |
| 24 | ALXN1210 LC | DIQMTQSPSSLSASVGDRVTITCGASENIYGALNWYQQKP |
| | | GKAPKLLIYGATNLADGVPSRFSGSGSGTDFTLTISSLQP |
| | | EDFATYYCQNVLNTPLTFGQGTKVEIKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQG |
| | | LSSPVTKSFNRGEC |
| 25 | CFZ533 HCDR1 | SYGMH |
| | (Kabat) | |
| 26 | CFZ533 HCDR2 | VISYEESNRYHADSVKG |
| | (Kabat) | |
| 27 | CFZ533 HCDR3 | DGGIAAPGPDY |
| | (Kabat) | |
| 28 | CFZ533 LCDR1 | RSSQSLLYSNGYNYLD |
| | (Kabat) | |
| 29 | CFZ533 LCDR2 | LGSNRAS |
| | (Kabat) | |
| 30 | CFZ533 LCDR3 | MQARQTPFT |
| | (Kabat) | |
| 31 | CFZ533 VH | QVQLVESGGGVVQPGRSLRLSCAASGFTFSSYGMHWVRQA |
| | | PGKGLEWVAVISYEESNRYHADSVKGRFTISRDNSKITLY |
| | | LQMNSLRTEDTAVYYCARDGGIAAPGPDYWGQGTLVTVSS |
| 32 | CFZ533 VL | DIVMTQSPLSLTVTPGEPASISCRSSQSLLYSNGYNYLDW |
| | | YLQKPGQSPQVLISLGSNRASGVPDRFSGSGSGTDFTLKI |
| | | SRVEAEDVGVYYCMQARQTPFTFGPGTKVDIR |
| 33 | CFZ533 HC | QVQLVESGGGVVQPGRSLRLSCAASGFTFSSYGMHWVRQA |
| | | PGKGLEWVAVISYEESNRYHADSVKGRFTISRDNSKITLY |
| | | LQMNSLRTEDTAVYYCARDGGIAAPGPDYWGQGTLVTVSS |
| | | ASTKGPSVFPLAPSSKSTSGGTAALGCLVKDYFPEPVTVS |
| | | WNSGALTSGVHTFPAVLQSSGLYSLSSWTVPSSSLGTQTY |
| | | ICNVNHKPSNTKVDKRVEPKSCDKTHTCPPCPAPELLGGP |
| | | SVFLFPPKPKDTLMISRTPEVTCVVVDVSHEDPEVKFNWY |
| | | VDGVEVHNAKTKPREEQYASTYRVVSVLTVLHQDWLNGKE |
| | | YKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSPGK |
| 34 | CFZ533 LC | DIVMTQSPLSLTVTPGEPASISCRSSQSLLYSNGYNYLDW |
| | | YLQKPGQSPQVLISLGSNRASGVPDRFSGSGSGTDFTLKI |
| | | SRVEAEDVGVYYCMQARQTPFTFGPGTKVDIRRTVAAPSV |
| | | FIFPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSFNRGEC |
| 35 | CD40 mAb2 HC | QVQLVESGGGVVQPGRSLRLSCAASGFTFSSYGMHWVRQA |
| | | PGKGLEWVAVISYEESNRYHADSVKGRFTISRDNSKITLY |
| | | LQMNSLRTEDTAVYYCARDGGIAAPGPDYWGQGTLVTVSS |
| | | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKRVEPKSCDKTHTCPPCPAPELLGG |
| | | PSVFLFPPKPKDTLMISRTPEVTCVVVAVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | TMKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| 36 | CD40 mAb2 LC | DIVMTQSPLSLTVTPGEPASISCRSSQSLLYSNGYNYLDW |
| | | YLQKPGQSPQVLISLGSNRASGVPDRFSGSGSGTDFTLKI |
| | | SRVEAEDVGVYYCMQARQTPFTFGPGTKVDIRRTVAAPSV |
| | | FIFPPSDEQLKSGTASVVCLLNNFYPREAKVQWKVDNALQ |
| | | SGNSQESVTEQDSKDSTYSLSSTLTLSKADYEKHKVYACE |
| | | VTHQGLSSPVTKSFNRGEC |
| 37 | Amino acid | MVRLPLQCVLWGCLLTAVHPEPPTACREKQYLINSQCCSL |
| | sequence of | CQPGQKLVSDCTEFTETECLPCGESEFLDTWNRETHCHQH |
| | CD40 | KYCDPNLGLRVQQKGTSETDTICTCEEGWHCTSEACESCV |
| | | LHRSCSPGFGVKQIATGVSDTICEPCPVGFFSNVSSAFEK |
| | | CHPWTSCETKDLVVQQAGTNKTDVVCGPQDRLRALWIPII |
| | | FGILFAILLVLVFIKKVAKKPTNKAPHPKQEPQEINFPDD |
| | | LPGSNTAAPVQETLHGCQPVTQEDGKESRISVQERQ |

The following examples illustrate the invention described above, but are not, however, intended to limit the scope of the invention in any way. Other test models known as such to the person skilled in the pertinent art can also determine the beneficial effects of the claimed invention.

### Results

### EXAMPLE 1

This example describes the binding affinities of the anti-CD40 antibodies CFZ533 and 2C10 to different species, e.g., pig-CD40, NHP-CD40 and human-CD40.

Methods

CFZ533 and CD21 Double Stain—FACS Sorting

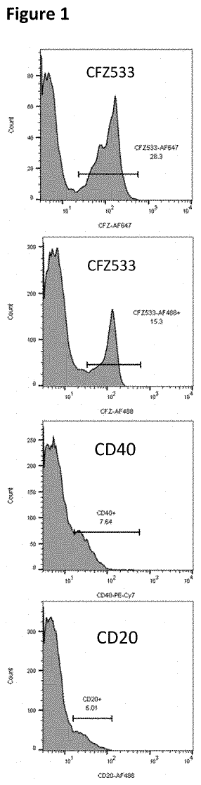

CFZ533 labelled using either an AF488 or AF647 Protein Labelling Kit (Invitrogen, #A10235 or #A20173, respectively). 200,000 cells were transferred to each FACS tube and samples washed with fresh FACS buffer (PBS+0.5% FBS+2 mM EDTA). Fc blocking was performed using 1:10 dilution of porcine serum (Life Technologies, #26250084), by incubation for 10 minutes at 4° C. Antibody staining was performed for 30 minutes at 4° C., in the dark with labelled CFZ533 and commercially available anti-CD21 (Southern Biotech, #4530-09) and anti-CD20 FACS antibodies (Novus Biologicals, #NBP1-44634). Samples were washed and resuspended in 200 ul of fresh FACS buffer for acquisition. Acquisition performed on LSR Fortessa (BD). The FIG. 1 shows that CFZ533 is able to bind to pig PBMC. Limited or no binding observed for our anti-human CD40 FACS antibody and our anti-pig CD20 FACS antibody.

MegaCD40L Dose Response
Pig PBMCs were thawed using a water bath and washed with fresh culture media (RPMI+Glutamax, 10% FBS, 1% HEPES, 1× Pen/Strep+0.05 mM β-mercaptoethanol)
1:3 dose response of human MegaCD40L (Enzo Life Sciences, #ALX-522-110-0010) was performed in culture media, with 75 ng/ml IL-4 (Thermo Fischer, #PSC0044), using a starting dose of 50 ug/ml.
Cells were added at 2×10\hat{ }5 cells/well, to plate already containing MegaCD40L dose response, and incubated for 72 h at 37° C.
Proliferation was measured by thymidine incorporation.

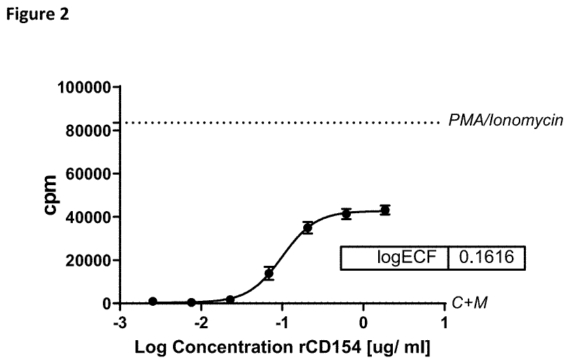

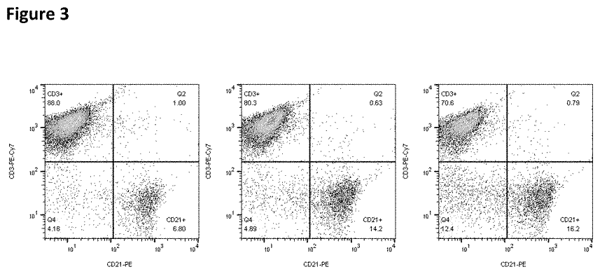

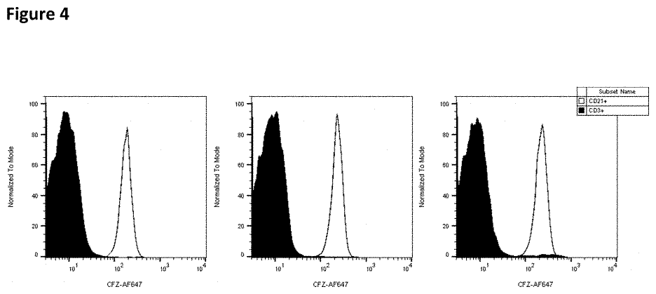

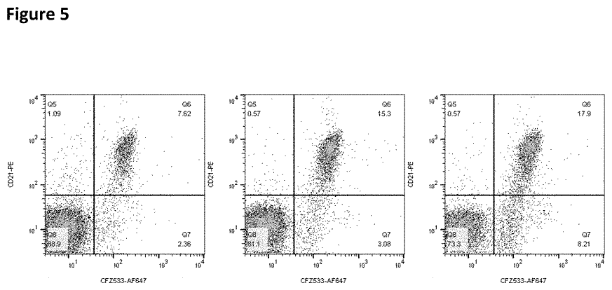

The plot in FIG. 2 shows that the pig cell selected using the CD21 marker +cells (B-cells) can be activated using human rCD154 (CD40L). MPG22951, MPG22949, MPG22950: Lot# of the PBMC vials; each lot being a different donor. FIG. 3 shows anti-pig CD21 FACS antibody selection using CD21 as a B-cell marker. As shown in the FIG. 3, B-cells and T-cells can be selected. FIG. 4 shows that the cell selected using the CD21 marker+cells (B-cells) bind CFZ533. This plot shown in FIG. 5 is another way of presenting the data of FIG. 4 showing that CFZ533 binds the cells marked using CD21 (B-cells).

Pig PBMC Binding and Proliferation Inhibition

Pig PBMCs were thawed using a water bath and washed with fresh culture media (RPMI+Glutamax, 10% FBS, 1% HEPES, 1× Pen/Strep +0.05mM 11-mercaptoethanol); a 1:3 dose response of CFZ533 was performed in culture media, with 75 ng/ml IL-4 (Thermo Fischer, #PSC0044), using a starting dose of 5 ug/ml. The Cells were added at 2×10\hat{ }5 cells/well, to plate already containing CFZ533 dose response, and pre-incubated for 1 h at 37° C. 2.5 ug/ml of MegaCD40L (Enzo Life Sciences, #ALX-522-110-0010) was added per well and plates were incubated for a further 18 h (or O/N). Proliferation was measured by thymidine incorporation.

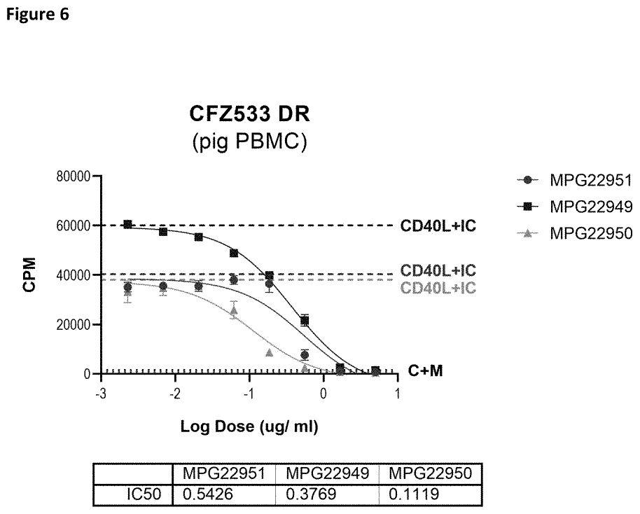

The plot shown in FIG. 6 shows that proliferation of pig PBMCs induced by the hCD40L can be inhibited by CFZ533; this is a functional prove that CFZ533 binds and blocks pig CD40.

### Experimental Results

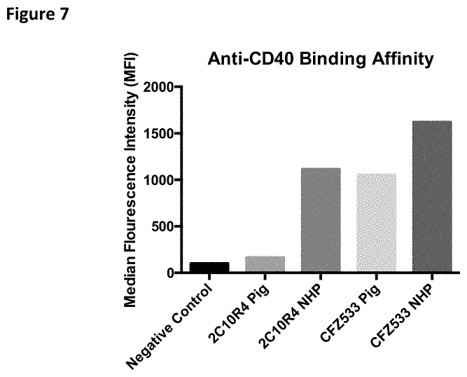

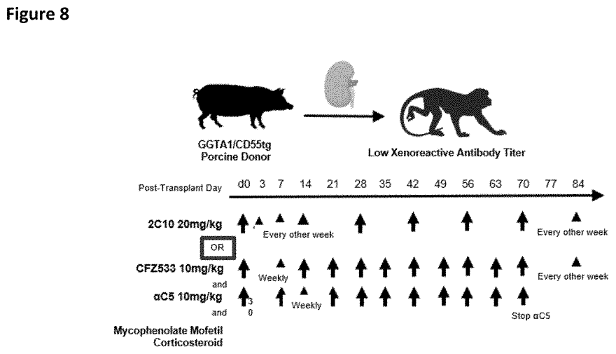

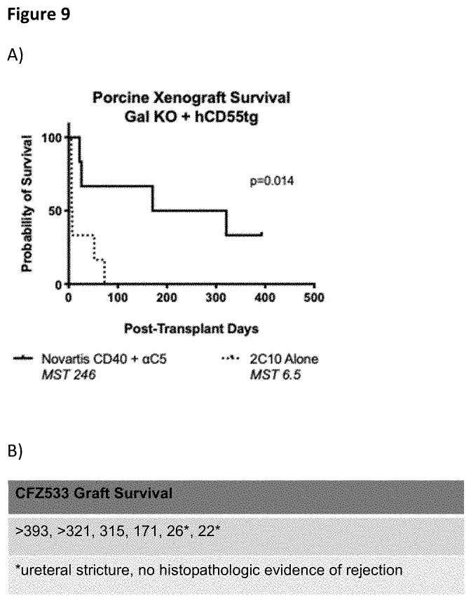

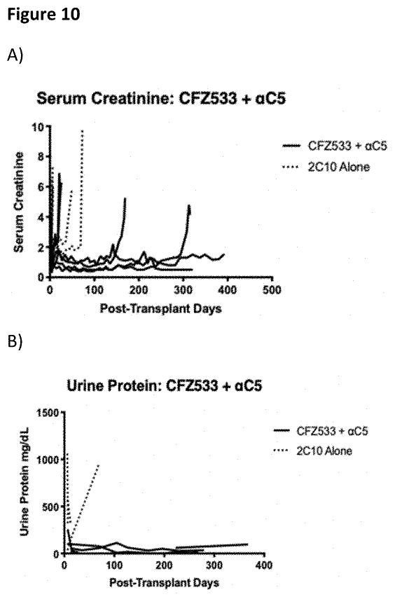

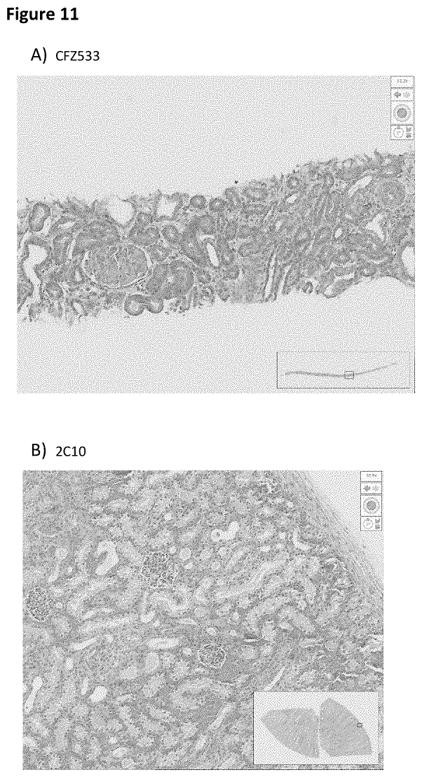

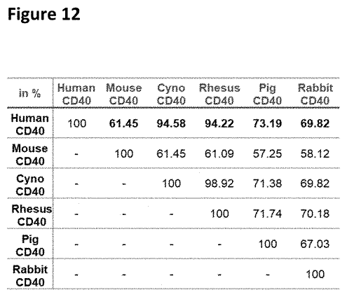

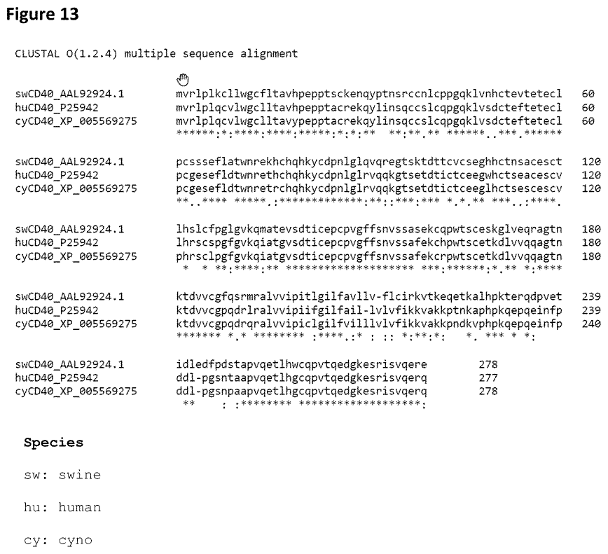

The experimental data provided in FIG. 7 clearly demonstrated that 2C10 has only a poor binding efficacy to porcine cells, whereas CFZ533 has superior binding efficacy to both porcine and non-human-primate (NHP) cells. This finding is furthermore supported by the data provided in FIGS. 1-6 clearly demonstrating that:
1. pig cell selected using the CD21 marker+cells (B-cells) can be activated using human rCD154 (CD40L)
2. CFZ533 binds to pig PBMCs
3. proliferation of pig PBMCs induced by the hCD40L can be inhibited by CFZ533, a functional prove that CFZ533 binds and blocks pig CD40.

In the patent application W02012065950 detailed experimental in vitro profiling data of CFZ533 (mAb1) have been provided. Example 9 of WO2012065950, which is herein incorporated by reference, discloses in table 10 a direct comparison of the binding EC50 values for CFZ533 in three species: human, Rhesus and Cynomolgus. CFZ533 binds to CD20+ cells (B-cells) of all three species with comparable EC50. Furthermore, as published previously, CFZ533 inhibited rCD154-induced proliferation of PBMCs from Cynomolgus monkeys (Cordoba et al., 2015). CFZ533 inhibited rCD154-induced proliferation of PBMCs from humans, rhesus and cynomolgus animals with similar potency (IC50 of 0.02, 0.03, and 0.01 μg/ml, respectively), and could also bind CD40 on B cells from these species with EC50 values of approximately 0.2 μg/ml, see table 2.

TABLE 2
Cellular binding and functional properties
of CFZ533 in human and NHPs.

| | Inhibition of rCD154-induced | | CD40 occupancy by CFZ533 | |
|---|---|---|---|---|
| | proliferation (IC50 PBMCs) | | (MFI EC50 on CD20+ cells) | |
| Human | 0.017 + 0.012 | μg/ml | 0.22 + 0.042 | μg/ml |
| | 0.12 + 0.08 | μM (n = 32) | 1.49 + 0.28 | μM (n = 4) |
| Rhesus | 0.026 + 0.017 | μg/ml | 0.22 + 0.033 | μg/ml |
| | 0.18 + 0.12 | μM (n = 8) | 1.49 + 0.22 | μM (n = 6) |
| Cynomol- | 0.010 + 0.003 | μg/ml | 0.20 + 0.068 | μg/ml |
| gus | 0.07 + 0.02 | μM (n = 4) | 1.35 + 0.46 | μM (n = 4) |

The above cellular data were derived from experiments where CFZ533 was added prior to, or simultaneously with rCD154, indicating that the antibody could prevent binding of the endogenous ligand.

### EXAMPLE 2

### Evaluation of the Agonistic Activity of Antibodies

Methods

Methods to prove the CD40L agonistic activity of antibodies and experimental data showing the non-agonistic activity of the antibodies CFZ533 and mAb2 are disclosed in the patent publication WO2012065950 in the example sections: 1. CD40L-mediated PBMC proliferation assay; 1.1 Purification of human peripheral blood mononuclear cells (PBMCs) and 1.2 In vitro PBMC stimulation assay, which are herewith incorporated by reference.

The experimental data based on the above-mentioned methods confirmed that the anti-CD40 antibodies CFZ533 (N297A) and mAb2 (D265A) showed non agonistic CD40L blocking properties.

In particular, the experimental results showed that none of the Fc silent anti-CD40 monoclonal antibodies (mAbs) were capable of stimulating cell division by human PBMCs (n=4 donors). PBMCs proliferated in response to CD40L. Collectively these results indicated that none of the anti-CD40 CFZ533 (N297A) and mAb2 (D265A) mAbs possessed agonistic activity. The above results also clearly demonstrated that CFZ533 and mAb2 did not have agonist activity in the presence of co-stimulatory signals.

### Experimental Results

The herein disclosed data indicate that CFZ533 is able to bind CD21+ B cells (but not CD3+ T cells) in pig PBMCs. Further we could demonstrate that recombinant human CD154 (CD40 ligand) was able to induce proliferation of pig PBMCs and this could be fully inhibited by CFZ533. These results indicate that CFZ533 is able to bind and prevent pathway activation downstream of pig CD40.

### EXAMPLE 3

This example describes a study to compare three different immunosuppression regimens and the results will be compared to historic controls. The animals will receive (i) an anti-C5 treatment and costimulatory blockade with anti-CD154 or an (ii) anti-C5 treatment and tacrolimus or (iii) an anti-C5 treatment and anti-CD40 antibody treatment. All animals will receive induction therapy with anti-CD4 and anti-CD20. In addition, all recipients will be treated with MMF and steroids.

Methods

Animals:

Porcine donors (˜15-50 kg), provided by University of Alabama, Birmingham, USA, are αGal−/−, β4Gal −/− double knock out pigs. Adolescent rhesus macaques (˜4-8 kg), provided by Yerkes Primate Center, Emory University, USA, have been utilized as recipients.

Surgical Technique:

The rhesus macaques renal transplant model is a well-characterized vascularized organ allograft model, which has been extensively used at the Yerkes Primate Center. Once anesthetized the macaques are clipped over the incision area and prepped with surgical scrub and alcohol. Surgeon prep included full scrubbing and gowning, and aseptic technique. Body temperature was maintained during surgery with IV fluid warmers, surgical table circulating water blankets, and pediatric operative forced air thermal blankets. The donor and recipient procedures were performed through a ventral midline laparotomy incision. The donor procedure involved mobilization of the renal artery and vein and mobilization of the ureter. Each structure was ligated with 5.0 silk and divided. The kidney was flushed with chilled solution (University of Wisconsin) for storage until implanted in the recipient. The animals were heparinized during organ harvest and implantation (100 units/kg). The xenograft was implanted using standard microvascular techniques to create an end to side anastomosis between the donor renal artery and recipient distal aorta with 8-0 prolene as well as the donor renal vein and recipient vena cava with 7-0 prolene. A primary ureteroneocystostomy was then created through a ventral midline cystotomy by implanting the transplant ureter in a posterior sub-mucosal tunnel. A single mucosal to mucosal stitch of 6-0 PDS was used to secure the ureter into the bladder. Nephrectomy of the remaining native kidney was completed prior to closure. Closure was performed for both the donor and recipient with interrupted 2-0 PDS sutures for the fascia and 4-0 Vicryl or similar absorbable subcuticular sutures for the skin. These sutures were dissolved and did not require removal. Dermal glue was also used for skin approximation. Where wound approximation felt to require non-absorbable sutures due to the wound healing effects of an immunosuppressive compound, 4-0 nylon was used for skin closure and the skin sutures were removed in 10-14 days. Postop warming was continued with the use of circulating water blankets or equivalent methods.

Pre-Op Therapeutics—Recipient:

Atropine (0.4 mg/ml) @ 0.1 mg/kg, IM; buprenorphine 0.02 mg/kg for pre-emptive analgesia, cefazolin @ 100 mg/kg, IV; and 1 chewable baby aspirin, 81 mg, crushed and placed into the cheek pouch (for anticoagulation).

Intra-Op Therapeutics—Recipient:

Furosemide @ 1 mg/kg, IV on reperfusion; mannitol (12.5 gm/50 ml) @ 0.2 gm/kg IV on reperfusion; heparin @ 100 units/kg, IV prior to cross clamp of recipient vena cava; 0.9% NaCl 300-500 ml IV.

Post-Op Therapeutics—Donor and Recipient:

0.9% NaCl 100-150 ml IV @ 6, 12, and 24 hours post-transplant; cefazolin @ 100 mg/kg, IM, for 3 days. Immediate postoperative care was provided by personnel from Veterinary Medicine at Yerkes. Experimental drugs were also administered by the veterinary staff with laboratory staff present for IV drug administration.

Postoperatively, animals were monitored for pain or distress (grimacing, splinting, withdrawn behavior) and were administered morphine 0.1 mg/kg IM every 4-6 hours for 24 hours and then buprenorphine 0.01-0.03 mg/kg every 6 hours, as needed in keeping with Yerkes guidelines.

Reagents:

Anti-C5 antibody (tesidolumab) and anti-CD40 antibody (iscalimab) were proved by Novartis. Anti-CD154 was purchased from the NHP Reagent Resource. MMF, steroid and other medications wer purchased from the McKesson Medical Supply.

Experimental Groups:

The anti-C5 antibody was tested with three different immunosuppression regimens and the results compared to historic controls. All animals received induction therapy with anti-CD4 and anti-CD20. In addition, all recipients were treated with MMF and steroids.

Group 1 (n=3)—anti-C5 treatment and costimulatory blockade with anti-CD154

Group 2 (n=3)—anti-C5 treatment and tacrolimus

Group 3 (n=3)—anti-C5 treatment and anti-CD40 antibody provided by Novartis.

Experimental Drug Dosing:

Anti-CD154—dosing 20 mg/kg on days 0, 3, 7, 14, 28 and every two weeks thereafter. Anti-C5—loading dose 30 mg/kg on the day of transplant, 10 mg/kg weekly thereafter×8 weeks. Anti-CD40—loading dose 30 mg/kg on the day of transplant, 10 mg/kg weekly thereafter×20 weeks.

Assessments

Outcomes included assessment of renal function based on serum creatinine, protocol biopsies performed at 2, 5, 10 and 20 weeks, flow cytometric analysis of leukocyte subsets, pharmacokinetic and immunogenicity analysis, evaluation of anti-pig antibody formation and viral reactivation assays including viral load assays for rhesus CMV, SV40, and LCV. In addition, plasma samples were collected to evaluate complement activity, using ELISA to evaluate C5b-9 levels.

Peripheral blood samples were used for immunophenotyping including flow cytometric analysis of T cell subsets and other cellular markers consistent with immune activation. In addition, peripheral blood samples (PAX gene tube) were drawn pre-transplant and on post-transplant days 7, 35, and 70 as well as at the time of sacrifice or suspected time of rejection. Urine samples were collected pre-transplant, on the day of transplant, POD 7, 14, 28, 42, 56 and monthly thereafter. Urine pellets were collected and stored for future batched RNA analysis while the urinary fluid was frozen for future batched proteomic analysis.

Protocol renal biopsies was performed on post-operative days 14, 35, 70, and 140 as well as at times of suspected rejection. Biopsies were analyzed using standard H&E, immunohistochemistry and banked for future gene expression analysis.

At the time of sacrifice renal xenograft was collected as well as peripheral blood, spleen, lymph node, and bone marrow samples for drug level, flow cytometric, and future gene expression analysis. Histology samples were collected and analysed including light microscopy (H&E) as well as immunohistochemistry. The renal xenograft was divided for histology as described above (frozen and fixed), and stored for future RNA isolation. RNA extracted from the biopsy, peripheral blood and sacrifice samples were stored for future batched gene array analysis using RNAseq. The remainder of the xenograft parenchyma was processed for extraction of tissue infiltrating cells, which was analyzed by flow cytometry. A necropsy inspection was performed by a staff veterinary pathologist. Standard issue samples were collected for histopathology (see list below). In addition, any tissue grossly abnormal at the time of necropsy was also be sent for histologic analysis.

Clinical Assessment and Recipient Survival:

Monitoring included a clinical assessment of urine output, nutritional intake, level of activity, etc. by Yerkes veterinarian staff. All recipients were assessed on a regular basis and treatment determined as necessary in conjunction with the Yerkes staff. Vital signs including temperature, blood pressure, pulse rate and weight were taken each time an animal was anesthetized for blood draws or medication administration. Unexplained episodes of illness were evaluated with blood cultures and further testing as indicated.

Graft function and laboratory assessment: Renal allograft function was monitored by measurement of serum BUN and creatinine, chemistries, and a CBC with differential on post-op days 4, 7, 14, 21, 28, and then at weekly intervals. Urine samples were collected by catheterization and stored for processing and including potential chemokine analysis. Xenograft failure was defined as the development of renal failure sufficient to require dialysis in a clinical setting (i.e., two consecutive values Cr>5/BUN>120 mg/dL, or any of the following associated with a rising creatinine: hyperkalemia>7.0, bicarbonate<12). Recipient survival time was recorded, and the animals euthanized at the time of xenograft failure. Animals judged to be severely ill by Yerkes veterinary staff (due to uremia or other causes) were euthanized. All recipients had a necropsy performed by Yerkes Veterinary Staff at the time of their deaths.

Protocol Renal Xenograft Biopsies:

Recipient animals underwent ultrasound guided percutaneous kidney biopsy on post-transplant days 14, 35, 70 and 140 or at the time of a suspected rejection episode. Percutaneous kidney biopsies were performed under telazol 3-5 mg/kg IM supplemented with ketamine as necessary. Once anesthetized, the kidney is palpated, and the area of skin over the kidney was clipped and prepped with surgical scrub and alcohol. Up to 3 samples were taken with a 20-gauge needle core device. Core biopsies were evaluated histologically including the characterization of the cellular infiltrate using immunohistochemistry and gene expression analysis (as described above). After the procedure animals were monitored for pain or distress (grimacing, splinting, withdrawn behaviour) and were administered buprenorphine 0.01-0.03 mg/kg every 6 hours, as needed in keeping with Yerkes guidelines.

Complement Activity-05b-9 ELISA:

Plasma samples for the evaluation of C5b-9 levels were measured at day 0 (pre-transplant),1, 4, 7, 14, 28. The plasma samples were frozen, stored, and batched at Emory for future analysis.

Anti-Pig Antibody Evaluation:

The development of anti-pig antibodies was measured in serum samples pre-transplant and then at days 28, 42 and 100 days and at the time of sacrifice. These samples were analyzed by flow-cytometry in the primate core lab to assess for the development of anti-pig antibody production including isotype analysis were appropriate.

Pharmacokinetic and Immunogenicity (Anti-Drug Antibody) Analysis:

Pre-dose serum samples were collected on the day of transplant and on post-transplant days 7, 14, 28, 42, 56, 70, 84, and 98. Samples were stored and sent for batched analysis by Novartis.

Viral Load Assays:

Animals were monitored for the presence of Rhesus cytomegalovirus (RhCMV), simian virus 40 (SV40), and lymphocryptovirus (LCV) by analyzing Rhesus whole blood using real-time PCR techniques previously described. Samples were taken pre-transplant and on days 0, 4, 7, 14, 28, 42, 56 and 70 post infusion/transplant. DNA was stored and batch analysis performed in the primate core lab.

Necropsy evaluation: Each animal had a formal necropsy performed by a Yerkes staff veterinary pathologist. A standard gross examination was performed. Tissues to be collected for examination included renal xenograft, mesenteric lymph node, para-aortic lymph nodes, and spleen. These samples were collected in 10% neutral buffered formalin or frozen in OCT compound. Any grossly abnormal tissue area was also collected, along with corresponding areas of tissue from control animals, where possible. Frozen and formalin-fixed tissues were sent for processing and histopathology/IHC evaluation.

Experimental Results

As disclosed herein, the combination immunosuppressive therapy with CFZ533 and tesidolumab significantly prolonged renal xenograft survival.

1.-11. (canceled)

12. A method of suppressing the rejection of a xenograft donor organ from an animal in a human recipient, the method comprising administering to the human recipient an anti-CD40 antibody, wherein the anti-CD40 antibody is an anti-CD40 antibody, or a functional fragment thereof, with silenced ADCC activity that binds both the xenograft and the human CD40 and said binding inhibits CD40L induced signaling with no or low agonist activity with respect to CD40 signaling.

13. The method of claim 12, wherein the xenograft donor organ is a pig organ and the anti-CD40 antibody or functional fragment thereof binds the pig CD40.

14. The method of claim 13, wherein the pig is a transgenic organism.

15. The method of claim 14, wherein the transgenic pig has been genetically modified and has disrupted a(1,3)-galactosyltransferase and CMAH genes.

16. The method of claim 12, wherein the anti-CD40 antibody or functional fragment thereof is selected from the group consisting of:
(a) an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
(b) an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
(c) an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34;
(d) an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36; and
(e) an anti-CD40 antibody, wherein the anti-CD40 antibody is iscalimab.

17. The method of claim 12, wherein the anti-CD40 antibody or functional fragment thereof is administered through a loading dose and/or a maintenance dose, and wherein the loading dosing consists of one, two, three or four intravenous administration(s) of a first dose and the maintenance dosing consists of weekly or biweekly subcutaneous injections of a second dose, and wherein the first dose is at least 10 mg and up to 30 mg anti-CD40 antibody or functional fragment thereof per kg of the recipient, followed by a maintenance dose which is between 300 mg and 600 mg.

18. The method of claim 17, wherein the loading dose of the anti-CD40 antibody or functional fragment thereof is administered at a dose of about 10 mg/kg to about 50 mg/kg per antibody.

19. The method of claim 18, wherein the loading dose of the anti-CD40 antibody or functional fragment thereof is administered as a single dose of about 10 mg/kg on the day of xenograft transplantation.

20. The method of claim 12, wherein the recipient is administered an induction therapy prior to receiving the xenotransplant.

21. The method of claim 20, wherein the induction therapy is administration of an anti-CD4 antibody and/or an anti-CD20 antibody.

22. The method of claim 12, wherein the anti-CD40 antibody treatment is combined with other anti-proliferative agents like mycophenolate mofetil (MMF), or steroids, like prednisone or T cell immunosuppressive compounds, like calcineurin inhibitors such as cyclosporin and tacrolimus.

23.-55. (canceled)

56. A method of suppressing the rejection of a xenograft organ from an animal in a human recipient, the method comprising administering to the human recipient an anti-C5 antibody and an anti-CD40 antibody or functional fragments thereof.

57. The method of claim 56, wherein a pharmaceutical composition comprising an anti-C5 antibody, or a functional fragment thereof and an anti-CD40 antibody, or a functional fragment thereof, in combination with at least a pharmaceutically acceptable excipient, carrier or diluent, is administered to the human recipient.

58. The method of claim 56, wherein the antibodies are co-administered using a fixed combination of the antibodies, or both antibodies are administered in parallel or sequentially using two different pharmaceutical compositions comprising each only one of the two antibodies through a loading dose and/or a maintenance dose.

59. The method of claim 58, wherein the fixed combination, or parallel or sequentially administered antibodies are administered through,
(a) a loading dose of the anti-CS-antibody, or a functional fragment thereof, at a dose of about 10 mg/kg to about 50 mg/kg per antibody and
(b) a loading dose of the anti-CD40 antibody, or a functional fragment thereof, at a dose of about 10 mg/kg to about 50 mg/kg per antibody.

60. The method of claim 56, wherein the loading dose of the anti-CS-antibody, or a functional fragment thereof, is administered as a single dose of about 10 mg/kg on the day of xenograft transplantation and the anti-CD40-antibody, or a functional fragment thereof, is administered as a single dose of about 10 mg/kg on the day of xenograft transplantation.

61. The method of claim 56, wherein the route of administration of the anti-C5 antibody, or a functional fragment thereof, is subcutaneous or intravenous, and/or wherein the administration the anti-CD40 antibody, or a functional fragment thereof, is subcutaneous or intravenous.

62. The method of claim 56, wherein the xenograft is a pig organ and the anti-CD40 antibody binds the pig CD40.

63. The method of claim 62, wherein the pig organ is from a transgenic organism.

64. The method of claim 63, wherein the transgenic pig has been genetically modified as follows: disrupted a(1,3)-galactosyltransferase and CMAH genes.

65. The method of claim 56, wherein the recipient is administered an induction therapy prior to receiving the xenotransplant.

66. The method of claim 65, wherein the induction therapy is administration of an anti-CD4 antibody and/or an anti-CD20 antibody.

67. The method of claim 56, wherein the anti-CD40 antibody is an anti-CD40 antibody selected from the group consisting of:
(a) an anti-CD40 antibody or functional fragment thereof comprising an immunoglobulin VH domain comprising the hypervariable regions set forth as SEQ ID NO: 25, SEQ ID NO: 26, and SEQ ID NO: 27 and an immunoglobulin VL domain comprising the hypervariable regions set forth as SEQ ID NO: 28, SEQ ID NO: 29 and SEQ ID NO: 30;
(b) an anti-CD40 antibody or functional fragment thereof comprising an immunoglobulin VH domain comprising the amino acid sequence of SEQ ID NO: 31 and an immunoglobulin VL domain comprising the amino acid sequence of SEQ ID NO: 32;
(c) an anti-CD40 antibody or functional fragment thereof comprising an immunoglobulin HC domain comprising the amino acid sequence of SEQ ID NO: 33 and an immunoglobulin LC domain comprising the amino acid sequence of SEQ ID NO: 34; and
(d) an anti-CD40 antibody or functional fragment thereof comprising an immunoglobulin HC domain comprising the amino acid sequence of SEQ ID NO: 35 and an immunoglobulin LC domain comprising the amino acid sequence of SEQ ID NO: 36.

68. The method of claim 56 claim 67, wherein the anti-CD40 antibody is iscalimab.

69. The method of claim 56, wherein the anti-C5 antibody is an antibody selected from the group consisting of:
(a) an anti-C5 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
(b) an anti-C5 antibody comprising an immunoglobulin VH domain comprising the amino acid sequence of SEQ ID NO: 7 and an immunoglobulin VL domain comprising the amino acid sequence of SEQ ID NO: 8;
(c) an anti-C5 antibody comprising an immunoglobulin VH domain comprising the hypervariable regions set forth as SEQ ID NO: 11, SEQ ID NO: 12, and SEQ ID NO: 13 and an immunoglobulin VL domain comprising the hypervariable regions set forth as SEQ ID NO: 14, SEQ ID NO: 15 and SEQ ID NO: 16;
(d) an anti-C5 antibody comprising an immunoglobulin VH domain comprising the amino acid sequence of SEQ ID NO: 17 and an immunoglobulin VL domain comprising the amino acid sequence of SEQ ID NO: 18.

70. The method of claim 69, wherein the anti-C5 antibody is tesidolumab or eculizumab.

71. The method of claim 56, wherein the anti-C5 antibody and anti-CD40 antibody treatment is combined with other anti-proliferative agents like mycophenolate mofetil (MMF), or steroids, like prednisone or T cell immunosuppressive compounds, like calcineurin inhibitors such as cyclosporin and tacrolimus.

72.-81. (canceled)


SEQUENCE LISTING (39 sequences)

SEQ ID NO: 1 - protein, 5 aa, Homo sapiens
SYAIS

SEQ ID NO: 2 - protein, 17 aa, Homo sapiens
GIGPFFGTANYAQKFQG

SEQ ID NO: 3 - protein, 7 aa, Homo sapiens
DTPYFDY

SEQ ID NO: 4 - protein, 11 aa, Homo sapiens
SGDSIPNYYVY

SEQ ID NO: 5 - protein, 7 aa, Homo sapiens
DDSNRPS

SEQ ID NO: 6 - protein, 11 aa, Homo sapiens
QSFDSSLNAEV

SEQ ID NO: 7 - protein, 116 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 8 - protein, 108 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 9 - protein, 446 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 5 aa, Homo sapiens
NYWIQ

SEQ ID NO: 12 - protein, 17 aa, Homo sapiens
EILPGSGSTEYTENFKD

SEQ ID NO: 13 - protein, 13 aa, Homo sapiens
YFFGSSPNWYFDV

SEQ ID NO: 14 - protein, 11 aa, Homo sapiens
GASENIYGALN

SEQ ID NO: 15 - protein, 7 aa, Homo sapiens
GATNLAD

SEQ ID NO: 16 - protein, 9 aa, Homo sapiens
QNVLNTPLT

SEQ ID NO: 17 - protein, 122 aa, Homo sapiens [sequence omitted]

SEQ ID NO: 18 - protein, 107 aa, Homo sapiens [sequence omitted]

SEQ ID NO: 19 - protein, 448 aa, Homo sapiens
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 214 aa, Homo sapiens [sequence omitted]

SEQ ID NO: 21 - protein, 448 aa, Homo sapiens
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 22 - protein, 214 aa, Homo sapiens [sequence omitted]

SEQ ID NO: 23 - protein, 448 aa, Homo sapiens
QVQLVQSGAEVKKPGASVKVSCKASGHIFSNYWIQWVRQAPGQGLEWMGEILPGSGHTEYTENFKDRVTNTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 24 - protein, 214 aa, Homo sapiens [sequence omitted]

SEQ ID NO: 25 - protein, 5 aa, Homo sapiens
SYGMH

SEQ ID NO: 26 - protein, 17 aa, Homo sapiens
VISYEESNRYHADSVKG

SEQ ID NO: 27 - protein, 11 aa, Homo sapiens
DGGIAAPGPDY

SEQ ID NO: 28 - protein, 16 aa, Homo sapiens
RSSQSLLYSNGYNYLD

SEQ ID NO: 29 - protein, 7 aa, Homo sapiens
LGSNRAS

SEQ ID NO: 30 - protein, 9 aa, Homo sapiens
MQARQTPFT

SEQ ID NO: 31 - protein, 120 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 32 - protein, 112 aa, Homo sapiens
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 33 - protein, 450 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 219 aa, Homo sapiens
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 450 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 219 aa, Homo sapiens; identical to SEQ ID NO: 34

SEQ ID NO: 37 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 38 - protein, 278 aa, Sus scrofa
MVRLPLKCLLWGCFLTAVHPEPPTSCKENQYPTNSRCCNLCPPGQKLVNHCTEVTETECLPCSSSEFLATWNREKHCHQHKYCDPNLGLQVQREGTSKTDTTCVCSEGHHCTNSACESCTLHSLCFPGLGVKQMATEVSDTICEPCPVGFFSNVSSASEKCQPWTSCESKGLVEQRAGTNKTDVVCGFQSRMRALVVIPITLGILFAVLLVFLCIRKVTKEQETKALHPKTERQDPVETIDLEDFPDSTAPVQETLHWCQPVTQEDGKESRISVQERE

SEQ ID NO: 39 - protein, 278 aa, Macaca fascicularis
MVRLPLQCVLWGCLLTAVYPEPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQRALVVIPICLGILFVILLLVLVFIKKVAKKPNDKVPHPKQEPQEINFPDDLPGSNPAAPVQETLHGCQPVTQEDGKESRISVQERQ